SEQUENCE LISTING (16 sequences)

SEQ ID NO: 1 - DNA, 20 bp, Artificial Sequence
  synthetic oligonucleotide
ggtcatctcccgattgaagt

SEQ ID NO: 2 - DNA, 20 bp, Artificial Sequence
  synthetic oligonucleotide
tcaatccaattggtaagcca

SEQ ID NO: 3 - DNA, 20 bp, Artificial Sequence
  synthetic oligonucleotide
tggagactgtgaaggtgctc

SEQ ID NO: 4 - DNA, 20 bp, Artificial Sequence
  synthetic oligonucleotide
tgagctttgtgggtaactgc

SEQ ID NO: 5 - DNA, 20 bp, Artificial Sequence
  synthetic oligonucleotide
agaattccgactggatcagg

SEQ ID NO: 6 - DNA, 20 bp, Artificial Sequence
  synthetic oligonucleotide
ggtcaccctcttcatcaggt

SEQ ID NO: 7 - DNA, 20 bp, Artificial Sequence
  synthetic oligonucleotide
ccgtcatcacctacatcagg

SEQ ID NO: 8 - DNA, 20 bp, Artificial Sequence
  synthetic oligonucleotide
agccagtctttcctttccct

SEQ ID NO: 9 - DNA, 18 bp, Artificial Sequence
  synthetic oligonucleotide
gtgacggtcggctttagg

SEQ ID NO: 10 - DNA, 19 bp, Artificial Sequence
  synthetic oligonucleotide
ggactgaggaggacgtgag

SEQ ID NO: 11 - DNA, 20 bp, Artificial Sequence
  synthetic oligonucleotide
cttscgcgaatgtttaggca

SEQ ID NO: 12 - DNA, 20 bp, Artificial Sequence
  synthetic oligonucleotide
gcatattagagccacaggca

SEQ ID NO: 13 - DNA, 11 bp, Artificial Sequence
  in silico generated consensus sequence
wawntrggtca

SEQ ID NO: 14 - DNA, 10 bp, Artificial Sequence
  in silico generated consensus sequence
rtkaygtaay

SEQ ID NO: 15 - DNA, 6 bp, Artificial Sequence
  synthetic oligonucleotide
cacgtg

SEQ ID NO: 16 - protein, 10 aa, Mus musculus
  ACETYLATION (7)..(7)
ASSPGGKILN

(19) United States
(12) Patent Application Publication
(10) Pub. No.: US 2011/0269817 A1
(43) Pub. Date: Nov. 3, 2011

(54) COMPOSITIONS AND METHODS RELATED TO SIRT1 FUNCTION

(75) Inventors: Paolo Sassone-Corsi, Laguna-Beach, CA (US); Yasukazu Nakahata, Ikoma (JP)

(73) Assignee: The Regents of the University of California, Oakland, CA (US)

(21) Appl. No.: 13/055,568

(22) PCT Filed: Jul. 24, 2009
(86) PCT No.: PCT/US2009/004291

§ 371 Date: Jul. 19, 2011

Related U.S. Application Data
(60) Provisional application No. 61/083,477, filed on Jul. 24, 2008.
(60) Provisional application No. 61/156,223, filed on Feb. 27, 2009.
(60) Provisional application No. 61/168,378, filed on Apr. 10, 2009.

(51) Int. Cl. A61K 31/713; C12Q 1/34; C07K 16/18; A61K 31/4545; A61K 31/498; A61P 11/06; A61K 31/496; A61P 25/00; A61P 3/00; A61P 25/24; A61P 9/00; C12N 9/99; A61K 31/429

(52) U.S. Cl. 514/44 A; 435/184; 435/18; 530/389.1; 514/318; 514/249; 514/368; 514/254.02

(57) ABSTRACT

The invention relates to modulation of circadian rhythm and underlying biological processes.

### RELATED APPLICATIONS

This application claims the benefit under 35 U.S.C. 119(e) of U.S. provisional application Ser. No. 61/083,477, filed Jul. 24, 2008, U.S. provisional application Ser. No. 61/156,223, filed Feb. 27, 2009, and U.S. provisional application Ser. No. 61/168,378, filed Apr. 10, 2009, the entire contents of each of which are incorporated by reference herein.

### GOVERNMENT SPONSORED RESEARCH

This invention was made with Government support under grants RO1-GM081634-01 awarded by the National Institutes of Health. The Government has certain rights to this invention.

### FIELD OF THE INVENTION

The invention relates to modulation of circadian rhythm and underlying biological processes.

### BACKGROUND OF INVENTION

The epigenetic basis of many developmental, physiological, and metabolic processes is manifest. Epigenetic mechanisms control gene expression by potentially reversible changes in DNA methylation and chromatin structure. The remodeling of chromatin is largely elicited by enzyme-catalyzed post-translational modifications of the core histone N-terminal tails (Kouzarides, 2007; Li et al., 2007a; Peterson and Laniel, 2004; Strahl and Allis, 2000). These include acetylation, poly(ADP-ribosylation), ubiquitination, methylation, and phosphorylation and represent critical regulatory events of a large array of nuclear responses. Unique combinations of these modifications, for which the “histone code” hypothesis has been formulated (Strahl and Allis, 2000), induce conformational changes of chromatin, rendering it permissive to transcription, silencing, DNA replication, and repair (Cheung et al., 2000a; Kouzarides, 2007; Kurdistani and Grunstein, 2003; Li et al., 2007a; Strahl and Allis, 2000).

Histone acetylation is recognized as one of the most prominent epigenetic marks leading to activation of gene expression (Strahl and Allis, 2000). Acetylation of the 3-amino groups of specific lysine residues in the N termini of core histones is generally associated with transcription activity, as it is thought to induce an open chromatin conformation that allows the transcription machinery access to promoters (Cheung et al., 2000a; Li et al., 2007a; Struhl, 1998). Indeed, acetylation of lysines in histones neutralizes their positive electric charge, thereby increasing repulsion within the negatively charged DNA backbone, which tips the balance toward chromatin relaxation. Deacetylation, on the other hand, would shift the balance back to condensing chromatin and silencing gene expression. The enzymes that elicit these critical transitions are histone acetyltransferases (HAT) and histone deacetylases (HDAC). HDAC-mediated deacetylation of histones correlates with gene silencing (Grunstein, 1997; Struhl, 1998; Wade and Wolffe, 1997; Workman and Kingston, 1998). HDACs have also been implicated in the reversible acetylation of nonhistone proteins, including p53 (Luo et al., 2001; Vaziri et al., 2001), Hsp90 (Kovacs et al., 2005), MyoD (Mal et al., 2001), and E2F1 (Martinez-Balbas et al., 2000). Mammalian HDACs have been classified into four classes based on their structure and regulation (Yang and Seto, 2008). There are seven mammalian enzymes constituting class III; these are homologs of yeast Sir2 (silencing information regulator) and are known as SIRT1 to SIRT7. These proteins are structurally distinct from the other HDACs and have the property of dynamically sensing cellular energy metabolism (Bordone and Guarente, 2005). Indeed, unlike other HDACs, SIRT proteins catalyze a unique reaction that requires the coenzyme NAD⁺ (nicotinamide adenine dinucleotide). In this reaction, nicotinamide (NAM) is liberated from NAD⁺ and the acetyl group of the substrate is transferred to cleaved NAD⁺, generating the metabolite O-acetyl-ADP ribose (Sauve et al., 2006). Due to the NAD⁺ dependency, SIRTs are thought to constitute one of the functional links between metabolic activity and genome stability and, finally, aging (Bishop and Guarente, 2007).

In yeast, the Sir2 complex mediates transcriptional silencing at telomeres and regulates the pace of aging (Chopra and Mishra, 2005; Oberdoerffer and Sinclair, 2007). Because of the NAD⁺ requirement for Sir2 deacetylase activity, it is evident that silencing is likely coupled to the metabolic cycle of cells. In C. elegans, one of the Sir2 orthologs, Sir2.1, has been shown to prevent aging (Tissenbaum and Guarente, 2001).

SIRT1, the mammalian ortholog of Sir2, is a nuclear protein that occupies a privileged position in the cell and governs critical metabolic and physiological processes. SIRT1 helps cells to be more resistant to oxidative or radiation-induced stress (Brunet et al., 2004; Luo et al., 2001), promotes mobilization of fat from white adipose tissues, an event that contributes to extending the life span (Picard et al., 2004), and mediates the metabolism of energy sources in metabolically active tissues (Lagouge et al., 2006; Rodgers et al., 2005). At the level of chromatin, SIRT1 enzymatic activity preferentially targets histone H3 at Lys9 and Lys14 and histone H4 at Lys16 (Imai et al., 2000). In addition, a number of nonhistone proteins, including p53 (Luo et al., 2001; Vaziri et al., 2001), FOXO3 (Brunet et al., 2004; Motta et al., 2004), PGC-1a (Nemoto et al., 2005; Rodgers et al., 2005), and LXR (Li et al., 2007a), are regulated by SIRT1-mediated deacetylation, stressing the pivotal function that this regulator plays in cellular control and responses.

### SUMMARY OF THE INVENTION

A remarkable array of metabolic and physiological processes display daily oscillations (Panda et al., 2002; Storch et al., 2002; Ueda et al., 2002), and an intimate interplay exists between circadian clocks and metabolic rhythms in all organisms (Wijnen and Young, 2006). The discovery that a core element of the circadian clock machinery, the protein CLOCK, is an enzyme with HAT activity (Doi et al., 2006) revealed the crucial role that chromatin remodeling plays in the circadian regulation of gene expression (Hardin and Yu, 2006; Nakahata et al., 2007). More recently, the finding that CLOCK specifically also acetylates nonhistone targets, such as its own partner BMAL1, suggested that it may control a number of physiological cellular functions (Hirayama et al., 2007). The intrinsic nature of SIRT1 as a NAD⁺-dependent HDAC prompted us to explore the possibility that SIRT1 could participate in circadian control by regulating the HAT function of CLOCK.

The invention is based, in part, upon the discovery that the HDAC activity of SIRT1 is regulated in a circadian manner in cultured cells and in the liver. SIRT1 physically associates with CLOCK and contributes to the acetylated state of CLOCK targets, such as Lys9/Lys14 in the tail of histone H3 and Lys537 in the BMAL1 protein. CLOCK, BMAL1, and SIRT1 colocalize in a chromatin-associated regulatory complex at promoters of clock-controlled genes. Pharmacological inhibition of SIRT1 activity by NAM and the drug splitomicin causes a loss in stringency of circadian gene expression, an effect equally observed in mouse embryo fibroblasts (MEFs) derived from Sirt1 null mice. Importantly, this effect is paralleled by a significant reduction in the oscillation of H3 and BMAL1 acetylation. Finally, using tissue-specific mutant mice, in which the Sirt1 gene is mutated uniquely in the liver, we demonstrate that SIRT1 contributes to circadian regulation in vivo. We propose that SIRT1 functions as an enzymatic rheostat of CLOCK function, thereby transducing signals originated by cellular metabolites to the circadian machinery.

According to one aspect of the invention, methods are provided that include administering to a subject having a disease or disorder associated with a circadian rhythm dysfunction and in need of such treatment an agent that modulates SIRT1 activity or expression or that modulates binding of SIRT1 to CLOCK or CLOCK/BMAL, in an amount effective to modulate the SIRT1 activity or expression or the binding of SIRT1 to CLOCK or CLOCK/BMAL. In some embodiments, the disease or disorder is a sleep disorder. In some embodiments, the sleep disorder is insomnia, jet lag, shift work sleep disorder, delayed sleep phase syndrome (DSPS), advanced sleep phase syndrome (ASPS), non 24-hour sleep wake disorder or irregular sleep-wake pattern. In some embodiments, disease or disorder is a psychiatric disorder associated with circadian rhythm. In some embodiments, the psychiatric disorder is depression. In some embodiments, the disease or disorder is a neurological disease with a circadian rhythm component. In some embodiments, the neurological disease is Alzheimer's disease. In some embodiments, the disease or disorder is anorexia nervosa. In some embodiments, the disease or disorder is abnormal blood pressure. In some embodiments, the disease or disorder is abnormal heart rate. In some embodiments, the disease or disorder is asthma. In some embodiments, the disease or disorder is a disease or disorder the treatment of which benefits from increasing or decreasing metabolite levels, such as levels of NAD, NAM or NMN. In some embodiments, treating comprises ameliorating symptoms of the disease or disorder.

In some embodiments, modulating comprises changing the amplitude of a molecular oscillation associated with the circadian clock. In some embodiments, the molecular oscillation is an activation and/or inhibition of gene expression and/or gene product function. In some embodiments, the activation and/or inhibition of gene expression and/or gene product function is mediated by a post-translational modification of a protein. In some embodiments, the post-translational modification is an acetylation, phosphorylation, and/or methylation of a protein. In some embodiments, the protein is BMAL1 or PER2. In some embodiments, the post-translational modification is acetylation of lysine 537 of BMAL1 and/or acetylation of PER2.

In some embodiments, the agent increases deacetylation of a member of the CLOCK/BMAL1 pathway. In some embodiments, the agent increases the binding of SIRT1 to a member of the CLOCK/BMAL1 pathway and/or SIRT1 deacetylase activity. In some embodiments, the increase is mediated by an increase in SIRT1 expression. In some embodiments, the agent is a non-naturally occurring compound, such as SRT1720, SRT2183, or SRT1460. In some embodiments, the agent is administered at selected times of day or at selected periods of the circadian rhythm. In some embodiments, the agent is administered in a form that releases at certain times, optionally in an extended release form, in a periodic release form, or using a pump. In some embodiments, the agent is administered in a form that releases alternating doses of SIRT1 activator and SIRT1 inhibitor (e.g., for resetting or normalizing circadian rhythm or for resetting the circadian rhythm to coincide with administration of the medication for the disease or disorder). In some embodiments, the methods include first testing the subject to determine if the subject's disease or disorder has a circadian rhythm component.

According another aspect of the invention, methods for treating a disease or disorder that has a circadian rhythm component are provided. The methods include determining whether the circadian rhythm of a subject is disrupted, and administering to the subject in need of such treatment an agent that modulates SIRT1 activity or expression or that modulates binding of SIRT1 to CLOCK or CLOCK/BMAL, in an amount effective to treat the disease or disorder.

In some embodiments, the disease or disorder is a sleep disorder. In some embodiments, the sleep disorder is insomnia, jet lag, shift work sleep disorder, delayed sleep phase syndrome (DSPS), advanced sleep phase syndrome, non 24-hour sleep wake disorder or irregular sleep-wake pattern. In some embodiments, the disease or disorder is a psychiatric disorder associated with circadian rhythm. In some embodiments, the psychiatric disorder is depression. In some embodiments, the disease or disorder is a neurological disease with a circadian rhythm component. In some embodiments, the neurological disease is Alzheimer's disease. In some embodiments, the disease or disorder is anorexia nervosa. In some embodiments, the disease or disorder is abnormal blood pressure. In some embodiments, the disease or disorder is abnormal heart rate. In some embodiments, the disease or disorder is asthma. In some embodiments, the disease or disorder is a metabolic disorder. In some embodiments, the metabolic disorder is diabetes, abnormal insulin secretion, abnormal plasma glucose levels, obesity, or metabolic syndrome. In some embodiments, the disease or disorder is cancer. In some embodiments, the disease or disorder is a disease or disorder the treatment of which benefits from increasing or decreasing metabolite levels, such as levels of NAD, NAM or NMN.

In some embodiments, treating comprises ameliorating symptoms of the disease or disorder. In some embodiments, the agent is a non-naturally occurring compound, such as SRT1720, SRT2183, or SRT1460. In some embodiments, the agent is administered at selected times of day or at selected periods of the circadian rhythm. In some embodiments, the agent is administered in a form that releases at certain times, optionally in an extended release form, in a periodic release form, or using a pump. In some embodiments, the agent is administered in a form that releases alternating doses of SIRT1 activator and SIRT1 inhibitor (e.g., for resetting or normalizing circadian rhythm or for resetting the circadian rhythm to coincide with administration of the medication for the disease or disorder).

According to another aspect of the invention, methods for altering a circadian rhythm of a subject are provided. The methods include administering to the subject in need of such treatment an amount of a SIRT1 modulator (activator or inhibitor) effective to alter the circadian rhythm of the subject. In some embodiments, the methods are used for treating disrupted sleep patterns of the subject. In some embodiments, the methods are used for initiating the onset of sleep or prolonging a period of sleep in the subject. In some embodiments, the methods are used for increasing the level of alertness in the subject. In some embodiments, the methods are used for extending wakefulness of the subject. In some embodiments, the methods are used for increasing the rate of metabolism of the subject.

In some embodiments, altering the circadian rhythm of the subject is increasing or decreasing the amplitude of the circadian rhythm of the subject, increasing or decreasing one or more periods of the circadian rhythm of the subject, or re-setting the circadian rhythm of the subject. In some embodiments, the SIRT1 modulator is a non-naturally occurring compound, such as SRT1720, SRT2183, or SRT1460.

According to yet another aspect of the invention, methods for extending or shortening one or more periods of a circadian rhythm of a subject are provided. The methods include administering to the subject in need of such treatment an effective amount of a SIRT1 modulator (activator or inhibitor) effective to extend or reduce one or more periods of the circadian rhythm of the subject. In some embodiments, the methods are used for treating disrupted sleep patterns of the subject. In some embodiments, the methods are used for initiating the onset of sleep or prolonging a period of sleep in the subject. In some embodiments, the methods are used for increasing the level of alertness in the subject. In some embodiments, the methods are used for extending wakefulness of the subject. In some embodiments, the methods are used for increasing the rate of metabolism of the subject. In some embodiments, the SIRT1 modulator is a non-naturally occurring compound, such as SRT1720, SRT2183, or SRT1460.

According to still another aspect of the invention, methods for increasing the effectiveness of a therapeutic compound for treating a disease or disorder are provided. The methods include administering to a subject in need of such treatment an agent that modulates SIRT1 activity or expression or that modulates binding of SIRT1 to CLOCK or CLOCK/BMAL, in an amount effective to modulate a circadian rhythm of the subject, whereby the effectiveness of the therapeutic compound is increased relative to the effectiveness of the therapeutic compound without the administration of the agent. In some embodiments, the disease or disorder is a sleep disorder. In some embodiments, the sleep disorder is insomnia, jet lag, shift work sleep disorder, delayed sleep phase syndrome (DSPS), advanced sleep phase syndrome, non 24-hour sleep wake disorder or irregular sleep-wake pattern. In some embodiments, the disease or disorder is a psychiatric disorder associated with circadian rhythm. In some embodiments, the psychiatric disorder is depression. In some embodiments, the disease or disorder is a neurological disease with a circadian rhythm component. In some embodiments, the neurological disease is Alzheimer's disease. In some embodiments, the disease or disorder is anorexia nervosa. In some embodiments, the disease or disorder is abnormal blood pressure. In some embodiments, the disease or disorder is abnormal heart rate. In some embodiments, the disease or disorder is asthma. In some embodiments, the disease or disorder is a metabolic disorder. In some embodiments, the metabolic disorder is diabetes, abnormal insulin secretion, abnormal plasma glucose levels, obesity, or metabolic syndrome. In some embodiments, the disease or disorder is cancer.

In some embodiments, treating comprises ameliorating symptoms of the disease or disorder. In some embodiments, the SIRT1 modulator is a non-naturally occurring compound, such as SRT1720, SRT2183, or SRT1460. In some embodiments, the agent is administered at selected times relative to administration of the therapeutic compound. In some embodiments, the agent is administered in a form that releases at certain times, optionally in an extended release form, in a periodic release form, or using a pump. In some embodiments, the agent is administered in a form that releases alternating doses of SIRT1 activator and SIRT1 inhibitor (e.g., for resetting or normalizing circadian rhythm or for resetting the circadian rhythm to coincide with administration of the medication for the disease or disorder).

In some embodiments, the methods include first testing the subject to determine if the subject's disease or disorder has a circadian rhythm component. In some embodiments, the increased effectiveness of the therapeutic compound is a greater response of the subject to the same or a lesser dose of the therapeutic compound, or the same response of the subject to a lesser dose of the therapeutic compound.

According to a further aspect of the invention, methods for modulating CLOCK acetylase activity or BMAL acetylation in a eukaryotic cell are provided. The methods include modulating the expression or activity of SIRT1. In some embodiments, the modulation of the expression or activity of SIRT1 is an increase in SIRT1 expression. In some embodiments, the increase of SIRT1 expression is produced by expressing exogenous SIRT1 in the cell or by increasing expression of endogenous SIRT1. In some embodiments, the modulation of the expression or activity of SIRT1 is a decrease in SIRT expression. In some embodiments, the decrease in SIRT1 expression is produced by contacting the cell with a molecule that interferes with SIRT1 expression. In some embodiments, the molecule that interferes with SIRT1 expression is a siRNA molecule. In some embodiments, the modulation of the expression or activity of SIRT1 is an increase in SIRT1 activity. In some embodiments, the increase of SIRT1 activity is produced by contacting the cell with a SIRT1 activator. In some embodiments, the modulation of the expression or activity of SIRT1 is a decrease in SIRT1 activity. In some embodiments, the decrease of SIRT1 activity is produced by contacting the cell with a SIRT1 inhibitor. In some embodiments, the modulation of BMAL acetylation is deacetylation of lysine 537 of BMAL.

According to another aspect of the invention, methods of identifying modulators of the circadian rhythm are provided. The methods include contacting a cell that expresses SIRT1, CLOCK and BMAL with a candidate molecule, and determining the level, activity or acetylation state of a biomarker indicating circadian rhythm activity, wherein if the level or acetylation state of the biomarker in the cell that has been contacted with the candidate molecule differs from a reference or control level of the level or acetylation state of the biomarker, then the candidate molecule is a modulator of the circadian rhythm. In some embodiments, the biomarker is SIRT1 expression. In some embodiments, the biomarker is CLOCK acetylase activity. In some embodiments, the biomarker is BMAL1 lysine 537 deacetylation. In some embodiments, the biomarker is PER2 deacetylation. In some embodiments, the candidate molecule is a small molecule chemical compound, such as a non-naturally occurring compound.

According to still another aspect of the invention, isolated antibodies that specifically bind to BMAL1 acetylated at lysine 537, or antigen binding fragments of such antibodies are provided.

According to yet another aspect of the invention, methods of modulating a pathway associated with a metabolic disease, DNA repair, cancer, or ageing, are provided. The methods include a step of ascertaining that the pathway is influenced by CLOCK:BMAL1/SIRT1, and a further step of modifying SIRT1 interaction with CLOCK. In some embodiments, the metabolic disease is diabetes. In some embodiments, the step of modifying SIRT1 interaction comprises a step of inhibiting or promoting binding of SIRT1 to CLOCK. In some embodiments, the step of modifying SIRT1 interaction comprises a step of increasing or reducing SIRT1 expression.

According to a further aspect of the invention, methods of modulating gene expression of a gene, the expression of which is at least in part controlled by CLOCK, are provided, comprising a step of modifying SIRT1 interaction with CLOCK.

According to some aspects of this invention, methods of modulating circadian gene expression of a gene, the expression of which is at least in part controlled by CLOCK, are provided, comprising a step of modifying SIRT1 interaction with CLOCK.

According to still another aspect of the invention, methods of modulating a process that is at least in part regulated by CLOCK:BMAL1 are provided. The methods include identifying the process in a cell as being regulated at least in part by CLOCK:BMAL1; and exposing the cell to an agent that modulates at least one of Nampt expression and Nampt activity at a concentration effective to modulate the at least one of Nampt expression and Nampt activity. In some embodiments, the modulation of the at least one of Nampt expression and Nampt activity is a reduction in the at least one of Nampt expression and Nampt activity. In some embodiments, the agent in a Nampt inhibitor. In some embodiments, the process is a metabolic process, a process associated with apoptosis, or a process associated with DNA repair.

According to another aspect of the invention, methods are provided that include administering to a subject having a disease or disorder associated with deregulated apoptosis and in need of such treatment an agent that inhibits Nampt expression or function, in an amount effective to inhibit the Nampt expression or function. In some embodiments, the disease or disorder is cancer or involves a deregulated, inappropriate or unwanted immune response. In some embodiments, the administration results in immunosuppression. In some embodiments, the administration results in sensitization to genotoxic agents. In some embodiments, the disease or disorder has a circadian rhythm component. In some embodiments, treating comprises ameliorating symptoms of the disease or disorder. In some embodiments, the agent is FK866. In some embodiments, the agent is a siRNA molecule that reduces expression of Nampt expression. In some embodiments, the agent is administered at selected times of day or at selected periods of the circadian rhythm. In some embodiments, the agent is administered in a form that releases at certain times, optionally in an extended release form, in a periodic release form, or using a pump. In some embodiments, the methods include first testing the subject to determine if the subject's disease or disorder has a circadian rhythm component.

These and other aspects of the invention, as well as various advantages and utilities will be more apparent with reference to the drawings and detailed description of the invention.

### BRIEF DESCRIPTION OF THE DRAWINGS

The accompanying drawings are not intended to be drawn to scale. For purposes of clarity, not every component may be labeled in every drawing.

FIG. 1. SIRT1 Deacetylase Activity Is Circadian in Serum-Shocked MEFs and in the Liver.

(A) Endogenous SIRT1 and BMAL1 expression in MEFs after serum shock were determined by western blot.

(B and C) Sirt1 (B) and Dbp (C) genes expression in liver was analyzed by quantitative PCR. The highest expression time point was set to 1.

(D) Endogenous SIRT1 proteins from livers of entrained mice at indicated time points were immunoprecipitated and subjected to deacetylation assays. The value at ZT15 was set to 1. Top panel shows the amount of immunoprecipitated SIRT1.

(E) Cell extracts prepared from serum-shocked MEFs at indicated time points were complemented with recombinant SIRT1 protein and acetylated p53 peptide as substrate and subjected to deacetylation assay. Value at time 0 was set to 1.

(F) Liver extracts prepared from entrained mice were complemented with recombinant SIRT1 protein and acetylated p53 peptide as substrate and subjected to deacetylation assay. Values at ZT15 were set to 1. All data presented are the means±standard errors of the mean (SEM) of three independent samples.

FIG. 2. SIRT1 Regulates Circadian mRNA Expression of dbp and per2 Genes

(A) dbp and clock mRNA expression levels in wild-type (WT) and Sirt1 null (Sirt1−/−) MEFs after serum shock were examined by RNase protection assay. Representative results are shown and experiments were done using four independent samples.

(B) dbp and per2 mRNA expression profiles in WT and Sirt1−/− MEFs after serum shock were analyzed by quantitative PCR. Time 0 value in WT MEF for each gene was set to 1. All data presented are the means±SEM of three independent samples.

(C) Relative increment/decrement of dbp and per2 gene expression. Plus values on vertical lines mean increment and minus values on vertical lines indicate decrement.

FIG. 3. Circadian Histone H3 (Lys9/Lys14) Acetylation at dbp TSS Is Dependent on SIRT1 Activity

(A) Dbp expression levels in MEFs either treated with SIRT1 inhibitors (10 mMNAM or 120 mM splitomicin) or not were analyzed by quantitative PCR. The value at time 20 in MEFs without treatment was set to 1.

(B-D) Crosslinked cell extracts were isolated from MEF without treatment (B) or with SIRT1 inhibitors, 10 mMNAM (C), or 120 mM splitomicin (D) at indicated time points after serum shock, subjected to ChIP assay, and analyzed by quantitative PCR with dbp TSS primers (See FIG. 4C). Results of semiquantitative PCR are also shown at the bottom of each experiment. The value at time 16 in MEFs without treatment was set to 1. All data presented are the means±SEM of three independent samples.

FIG. 4. SIRT1 Is Recruited to the E-box and Regulates Circadian Histone Acetylation on the Dbp Gene

(A and B) Histone H3 (Lys9/Lys14) acetylation at Dbp TSS is hyperacetylated in SIRT1-deficient MEFs. Crosslinked cell extracts were isolated from WT or SIRT1-deficient MEFs at indicated time points after serum shock, subjected to ChIP assay with anti-acetyl histone H3 (Lys9/Lys14) and control IgG, and analyzed by semiquantitative PCR (A) or quantitative PCR (B) with TSS primers. Control IgG was used as a control for immunoprecipitation. The value at time 16 in WT MEF was set to 1.

(C) Schematic diagram of the mouse Dbp promoter and primers used for ChIP assay.

(D) Representative results of the ChIP assay analyzed by semiquantitative RT-PCR. Dual crosslinked cell extracts were isolated from MEF after 16 or 24 hr serum shock and subjected to ChIP assay with anti-SIRT1, anti-CLOCK, anti-BMAL1, or no antibody (ctrl). No antibody and 30R primers were used as controls for immunoprecipitation and PCR, respectively.

(E) Quantification of ChIP by quantitative PCR. Quantitative PCR was performed on the same samples as described in (D). All data presented are the means±SEM of three independent samples.

FIG. 5. Interaction of SIRT1 with CLOCK

(A) JEG3 cells were cotransfected with a series of expression vectors as described. Flag-tagged SIRT1 proteins were immunoprecipitated by FLAG antibody, and abundance of coimmunoprecipitated proteins was determined by western blotting using anti-Myc antibody (top right panel) and anti-SIRT1 antibody (bottom right panel). Left panels show the immunoblotting results of total cell lysates as an input.

(B and C) SIRT1 and CLOCK interaction in vivo. Endogenous CLOCK:BMAL1 and SIRT1 interactions in the mice liver (B) and in cultured MEFs after serum shock (C) were determined by coimmunoprecipitation assays.

(D) The deacetylase-deficient (H363Y) mutant SIRT1 interacts with CLOCK as well as WT SIRT1.

(E) CLOCK (aa 450-570) is required for the interaction with SIRT1. In vitro-translated [³⁵S]-labeled truncated CLOCK proteins were pulled down by GST-SIRT1.

(F) SIRT1 N terminus (aa 1-231) is required for CLOCK interaction. In vitro-translated [³⁵S]-labeled full-length CLOCK proteins were pulled down by truncated GST-SIRT1.

FIG. 6. SIRT1 Regulates BMAL1 Lys537 Acetylation

(A) Class III HDAC inhibitor enhances BMAL1 Lys537 acetylation. JEG3 cells transfected with Myc-CLOCK and Flag-Myc-BMAL1 were treated with HDAC I and II inhibitor, TSA (1 mM), for 6 hr and HDAC III inhibitor, NAM (10 mM), for 16 hr before harvest. Immunoprecipitated BMAL1 proteins by FLAG antibody were subjected to SDS-PAGE and probed with acetylated BMAL1 or Myc antibodies.

(B) SIRT1 deacetylates acetylated Lys537 in BMAL1. JEG3 cells transfected with Myc-CLOCK and Flag-Myc-BMAL1 were cotransfected with SIRT1-Flag, SIRT2-Flag, or SIRT3-Flag. Immunoprecipitated BMAL1 proteins by FLAG antibody were probed with acetylated BMAL1 or Myc antibodies. Immunoprecipitated SIRT proteins by FLAG antibody were probed with FLAG M2 antibody.

(C) Acetylated BMAL1 is not deacetylated by deacetylase-deficient mutant SIRT1. JEG3 cells transfected with Myc-CLOCK and Flag-Myc-BMAL1 were cotransfected with WT or mutant (H363Y) SIRT1-HA. Immunoprecipitated BMAL1 proteins by FLAG antibody were probed with acetylated BMAL1 or Myc antibodies. SIRT1 protein amount was detected by SIRT1 antibody.

(D) Deacetylation of BMAL1 by SIRT1 is NAD⁺ dependent manner. JEG3 cells transfected with Myc-CLOCK and Flag-Myc-BMAL1 and SIRT1-HA were treated with 1 mM NAD⁺ or 10 mM NAM for 16 hr before harvest. Immunoprecipitated BMAL1 proteins by FLAG antibody were probed with acetylated BMAL1 antibody. SIRT1 protein amount was detected by SIRT1 antibody.

FIG. 7. Circadian Dysfunction and BMAL1 Upregulated Acetylation in Liver-Specific SIRT1-Deficient Mice

(A) BMAL1 Lys537 acetylation profile in WT or SIRT1-deficient MEFs was investigated. Cell extracts prepared from indicated time points were immunoprecipitated with BMAL1 antibody and acetylation of BMAL1 was detected by probing with the acetylated BMAL1 antibody.

(B) BMAL1 Lys537 acetylation profile in WT and SIRT1 Dex4 mice was investigated. Liver extracts prepared from indicated time points were immunoprecipitated by BMAL1 antibody and acetylation of BMAL1 was detected by using the acetylated BMAL1 antibody. Acetylation of BMAL1 in the SIRT1-Dex4 mutant mice is nonrhythmic and elevated compared to WT mice. Overall levels of BMAL1 are also higher in the SIRT1-Dex4 mutant mice. The pattern of BMAL1 phosphorylation is also altered (see also FIG. 12). The lower SIRT1 band corresponds to the SIRT1-Dex4 deletion. Levels of the CLOCK protein and actin were used as control.

(C) Altered circadian expression of Cry1 and Per2 clock genes in the livers of SIRT1 Dex4 mice. Error bars represent SEM.

(D) Scheme of the NAD⁺-dependent regulation exerted by SIRT1 on the circadian clock machinery. SIRT1 interacts with CLOCK and thereby establishes a functional and molecular link between energy metabolism and circadian physiology.

FIG. 8

To assess whether SIRT1 protein levels oscillate in a circadian manner, nuclear extracts were prepared following various methods from mouse liver. The protein levels were monitored using a Western analysis using specific anti-SIRT1 antibodies (see Experimental Procedures). Independently from the protocol used, SIRT1 levels displayed marginal or no oscillation.

FIG. 9. Effect of different treatments on the CLOCK-SIRT1 interaction.

The effect of various agents on the CLOCK-SIRT1 interaction was analyzed. None of these treatments used significantly affected the efficacy of the interaction. JEG3 cells were cultured in BME containing 10% FBS and transfected with Myc-CLOCK, Myc-BMAL1, and Flag-SIRT1 constructs. Negative control (NC) represents cells transfected only with Myc-CLOCK and Myc-BMAL1. 24 h after transfection cells were replated, allowed to readhere for another 16 hours, and treated with indicated reagents for 1, 2 and 6 hours. Here the results with 6 h treatment are shown, the same results were obtained at 1 h and 2 h. Nuclear extracts were processed as previously described (Hirayama et al. 2007). This experiment was performed 3 times and a representative result is shown.

Legend: Lane 1: control (C); lane 2: 50% horse serum (HS); lane 3: 1 mM NAD⁺ (NAD⁺); lane 4: 10 mM glucose (Glc); lane 5: 1 mM pyruvate (Pyr); 6: 100 μM DFO (DF); lane 7: 100 μM resveratrol; (Res) lane 8:120 μM splitomicin (Split). Lane 9: negative control (NC).

FIG. 10. A Specific Antibody that Recognizes BMAL1 only when it is Acetylated at K537.

We have raised a polyclonal antibody against a 11 aa peptide in which K537 was uniquely acetylated. The antibody has been successfully tested in Western analysis, immunoprecipitations and immunohistochemistry. Here we show results of a transfection in 293 cells in which CLOCK, BMAL1 or a BMAL1(K537R) mutant are ectopically expressed. Western analysis shows that the antibody specifically recognizes BMAL1 when it is acetylated and that CLOCK elicits this event (as indeed recently demonstrated in our laboratory (Hirayama et al 2007).

FIG. 11.

SIRT1 deacetylates BMAL1 (K537) in vitro. For this experiment we used an antiacetyl-BMAL1 antibody developed in our laboratory (see FIG. 10). Acetylated BMAL1 was prepared from HDAC inhibitors-treated JEG3 cultured cells transfected with Flag-Myc-BMAL1 and Myc-CLOCK. Recombinant SIRT1 and deacetylation buffer were used from SIRT1 Fluorimetric Activity Assay/Drug Discovery Kit (AK-555; BIOMOL International). Immunoprecipitated Ac-BMAL1 and recombinant SIRT1 were incubated in deacetylation buffer with 5 mM NAD⁺ or 10 mM nicotinamide (NAM) for 90 min at 37° C. Reactions were stopped by adding SDS-PAGE denaturing buffer. Samples were subjected to SDS-PAGE and probed with Ac-BMAL1 or Myc antibodies.

FIG. 12.

This shows a lower exposure of the panel presented in FIG. 7B. This allows to better visualize BMAL1 phosphorylated and non-phosphorylated bands. While BMAL1 protein appears always to be more abundant—possibly indicating increased stability—in SIRT1-null cells, overall the pattern of phosphorylation doesn't change in the absence of SIRT1. The only time point where there may be a significant difference is at ZT21. In livers from WT mice the non-phosphorylated band appears to be prominent, whereas the bands corresponding to the phosphorylated the non-phosphorylated BMAL1 appears to equally represented in the livers from the SIRT1Δex4 mice.

FIG. 13

Panels A and B shows that cellular NAD⁺ was extracted from serum-entrained MEFs derived from wild type (wt) (A) and c/c mutant (B) mice at indicated time points and analyzed by LC/MSn. Three independent experiments were performed and representative results are shown. All data presented are the means±SEM of three independent samples. Panel C depicts average NAD⁺ levels in wt and c/c MEFs. Data from (A) and (B) are averaged and shown as the means±SEM of >50 independent samples. Panel D: Cellular nicotinamide (NAM) was extracted from serum-entrained MEFs derived from wt and c/c mutant mice at indicated time points and analyzed by LC/MSn. Three independent experiments were performed and representative results are shown. All data presented are the means±SEM of three independent samples. Panel E shows average NAM levels in wt and c/c MEFs. Data are shown as the means±SEM of >50 independent samples.

FIG. 14

This depicts circadian clock control of Nampt gene expression. Panel A is a schematic of the NAD⁺ salvage pathway in mammals. NAM, nicotinamide; NMN, nicotinamide mononucleotide; Nampt, nicotinamide phosphoribosyltransferase; Nmnat1-3, nicotinamide mononucleotide adenyltransferase. Panel B shows data on Nampt gene expression in livers from light-entrained wt and c/c mutant mice was quantified by q-PCR. Nampt gene expression at ZT 15 in liver from wt mice was set to 1. All data presented are the means±SEM of three independent samples. Panel C shows Nampt and Dbp gene expressions in serum-entrained MEFs from wt and c/c mutant mice were quantified by q-PCR. The expression at time 0 in wt MEFs was set to 1. All data presented are the means±SEM of three independent samples.

FIG. 15

This illustrates regulation of the Nampt promoter by CLOCK:BMAL1 and SIRT1. Panel A shows a schematic diagram of regulatory elements in human Nampt promoter. TSS (Transcription Start Site) primer region for q-PCR is shown as arrow heads. Arrows show the positions indicating truncated forms of Nampt promoter used in panel C. Transcription start site is marked at +1. Other putative transcription factors binding sites are indicated: HRE, hypoxia-inducible factor-responsible element; SP1, specificity protein 1; CRE, cAMP-response element; AP-1, activator protein 1; GRE, glucocorticoid receptor response element. Panel B illustrates Conserved E-boxes (bold capital letters) among species are shown. Numbers are the position from human transcription start site. Panel C depicts schematic diagram of different Nampt promoter constructs are shown on the left. The effects of CLOCK:BMAL1 (+CL/BM; black bars) on luciferase activity are shown on the right. The luciferase activity of CLOCK:BMAL1 on the pGL4.10 was set as 1. All data presented are the means±SEM of three independent samples. Panel D shows representative results of the ChIP assay analyzed by semiquantitative PCR. Dual crosslinked nuclear extracts were isolated from MEFs after 16 or 24 hr serum shock and subjected to ChIP assay with anti-SIRT1, anti-CLOCK, anti-BMAL1, or no antibody (ctrl). No antibody and 3′R primers were used as controls for immunoprecipitation and PCR, respectively. Panel E is a graph depicting quantification of ChIP by q-PCR. q-PCR was performed on the same samples as described in panel D. All data presented are the means±SEM of three independent samples.

FIG. 16

This illustrates Nampt modulation of the circadian clock. Panel A depicts Pert and Dbp gene expression levels in serum-entrained MEFs treated with 10 nM FK866 or EtOH as control (solvent:ctrl) were analyzed by q-PCR. The highest value for each gene in EtOH treated MEFs was set to 1. All data presented are the means±SEM of three independent samples. Panel B shows BMAL1 Lys537 acetylation profile in serum-entrained MEFs either treated with 10 nM FK866 or EtOH as control (solvent:ctrl) was investigated. Cell extracts prepared from indicated time points were processed to visualize BMAL1 acetylation using the anti-acetyl specific BMAL1 antibody as described herein. Input samples were probed with anti-BMAL1 and anti-GAPDH antibodies. Panel C is a schematic representation of the transcription-enzymatic interplay by which the circadian machinery governs the intracellular levels of NAD⁺. The NAD⁺-dependent deacetylase SIRT1 is thereby controlling the oscillatory synthesis of its own coenzyme.

FIG. 17

This depicts cellular NAD⁺ in c/c mutant MEFs. Cellular NAD⁺ was extracted from serum-entrained c/c mutant MEFs at indicated time points and analyzed by LC/MSn. Three independent experiments were performed and representative is shown. All data presented are the means±SEM of three independent samples.

FIG. 18

This illustrates results from Nmnat1-3 gene expressions in mice liver. Nampt1-3 gene expressions in liver from light-entrained mice were quantified by q-PCR using same sample as shown in FIG. 13C. The highest expression time point was set to 1. All data presented are the means±SEM of three independent samples.

FIG. 19 shows the effect of SRT2183 on circadian clock expression.

FIG. 20 shows the effect of SRT1720 on circadian clock control.

### DETAILED DESCRIPTION OF THE INVENTION

Circadian rhythms govern a large array of metabolic and physiological functions. The central clock protein CLOCK has HAT properties. It directs acetylation of histone H3 and of its dimerization partner BMAL1 at Lys537, an event essential for circadian function. We show that the HDAC activity of the NAD⁺-dependent SIRT1 enzyme is regulated in a circadian manner, correlating with rhythmic acetylation of BMAL1 and H3 Lys9/Lys14 at circadian promoters. SIRT1 associates with CLOCK and is recruited to the CLOCK:BMAL1 chromatin complex at circadian promoters. Genetic ablation of the Sirt1 gene or pharmacological inhibition of SIRT1 activity lead to disturbances in the circadian cycle and in the acetylation of H3 and BMAL1. Finally, using liver-specific SIRT1 mutant mice we show that SIRT1 contributes to circadian control in vivo.

Thus, we have discovered that SIRT1 functions as an enzymatic rheostat of circadian function, transducing signals originated by cellular metabolites to the circadian clock. Such finding has been supported by various data shown herein. Consequently, it should be appreciated that based on the connection between circadian metabolism, aging, and cancer via CLOCK:BMAL1/SIRT1, new regulatory pathways for conditions and diseases associated aging, metabolism, and cancer can be discovered and targeted with compounds that interfere with such pathways and/or CLOCK:BMAL1/SIRT1.

Treatment methods are provided in accordance with the invention. In one aspect, such methods include administering to a subject having a disease or disorder associated with a circadian rhythm dysfunction and in need of such treatment an agent that modulates SIRT1 activity or expression or an agent that modulates binding of SIRT1 to CLOCK or CLOCK/BMAL. The amount of agent administered is effective to modulate the SIRT1 activity or expression or to modulate the binding of SIRT1 to CLOCK or CLOCK/BMAL. Treating as used in this context includes ameliorating symptoms of the disease or disorder.

In these methods, modulating activity, expression or binding as described herein includes changing the amplitude of a molecular oscillation associated with the circadian clock. The molecular oscillation can be an activation and/or an inhibition of gene expression and/or gene product function. Activation and/or inhibition of gene expression and/or gene product function can be mediated by a post-translational modification of a protein, such as acetylation, phosphorylation, and/or methylation of a protein. Examples of this include acetylation of BMAL1 or PER2, or, more generally, of polypeptides acetylated by CLOCK. For example, acetylation of BMAL1 can be acetylation of lysine 537 of BMAL1.

The agents used in the methods can increase (or decrease) acetylation/deacetylation of a member of the CLOCK/BMAL1 pathway. For example, as shown herein, SIRT1 modulates CLOCK HAT activity, and therefore modulation of SIRT1 activity can be used to increase (or decrease) acetylation/deacetylation of a member of the CLOCK/BMAL1 pathway.

The agents used in the methods also can increase the binding of SIRT1 to a member of the CLOCK/BMAL1 pathway. This may be the result of increasing SIRT1 levels in a cell, such as by increasing SIRT1 expression, or by stabilizing the binding of SIRT1 to a member of the CLOCK/BMAL1 pathway.

Examples of agents that can be used to modulate the various activities are described elsewhere herein. For example, modulators of SIRT1 activity are known in the art and can be used in the methods of the invention.

Another feature of the invention is that the agent can be administered at selected times of day or at selected periods of the circadian rhythm to favorably influence the effect of the agent. For example, it may be preferred to administer the agent at or near the same time each day, thereby producing or enhancing regularity in circadian rhythm, or for re-setting a normal rhythm, for example during a period of jet lag. Likewise, it may be preferred to administer the agent at or near a period of wakefulness or alertness, to extend such a period and/or to increase the level of wakefulness or alertness. Agent also can be administered at periods of low wakefulness or alertness in order to shorten such periods. The schedule of administration may depend on a subject's chronotype, circadian type, diurnal preference or diurnal variation.

The agent can also be administered in a dosage form that releases the agent from the dosage form for an extended period of time or at certain selected times. For example, the agent can be administered in an extended release form, using a pump, or in a periodic release form, such as a formulation that uses coatings or materials that erode sequentially to deliver sequential doses of one or more agents, etc.). For example, the agent can be administered in a form that releases alternating doses of SIRT1 activator and SIRT1 inhibitor. This type of dosage form can be particularly useful for, e.g., resetting or normalizing circadian rhythm or for resetting the circadian rhythm to coincide with administration of another medication that is used for treating a disease or disorder in the subject.

Circadian rhythms may become desynchronized in various diseases or disorders, such as the diseases and disorders described herein, or in infectious diseases. The methods described herein permit altering the circadian rhythm of a subject, including increasing or decreasing the amplitude of the circadian rhythm, increasing or decreasing one or more periods of the circadian rhythm, and re-setting circadian rhythm of a subject. In such methods, a subject needing such treatment is administered an amount of a SIRT1 modulator (i.e., an activator or inhibitor) that is effective to alter the circadian rhythm of the subject.

Altering the circadian rhythm of a subject is useful in a variety of contexts, such as for treating sleep disorders, include adjusting and/or correcting disrupted sleep patterns of the subject or initiating the onset of sleep or prolonging a period of sleep in the subject. Alternatively, the methods can be used for increasing the level of alertness or extending wakefulness in the subject. The methods also can be used for increasing the rate of metabolism of the subject, including by altering the levels of NAD, NAM and/or NMN.

In addition to altering circadian rhythms, the methods described herein can be used to extend or shorten one or more periods of a circadian rhythm of a subject. Such methods can be used to treat disrupted sleep patterns of the subject, to initiate the onset of sleep or to prolong a period of sleep in the subject. The methods also can be used for increasing the level of alertness or extending wakefulness in the subject. The methods also can be used for increasing the rate of metabolism of the subject, including by altering the levels of NAD, NAM and/or NMN.

As described in more detail below, it has been found that intracellular NAD⁺ levels cycle with a 24 h rhythm, an oscillation driven by the circadian clock. CLOCK:BMAL1 regulate the circadian expression of Nampt (nicotinamide phosphoribosyltransferase), a rate limiting step enzyme in the NAD⁺ salvage pathway. SIRT1 is recruited to the Nampt promoter and contributes to the circadian synthesis of its own coenzyme. Using the specific inhibitor FK866, it was found that Nampt is required to modulate circadian gene expression as well as BMAL1 circadian acetylation. Based on these findings, it is particularly contemplated that an interlocked transcriptional-enzymatic feedback loop governs the molecular interplay between cellular metabolism and circadian rhythms. Accordingly, in addition to using agents that modulate sirtuin activity or expression, other agents that modulate other elements of this interlocked feedback loop can be used in a similar manner, and therefore can be utilized in the same, complementary, or opposing manner as the sirtuin modulators described herein.

Thus, administration of an modulator of Nampt expression or activity can be used for treating diseases or disorders associated with deregulated apoptosis, such as cancer, a disease or disorder involving a deregulated, inappropriate or unwanted immune responses, and the like.

For example, administration of an modulator of Nampt expression or activity can be used for modulating immune responses, such as for immunosuppression. Accordingly, the compounds described herein can be used as immunosuppressant compounds, which may be administered together with rapamycin or other immunosuppressant compounds to increase the effect of rapamycin or the other immunosuppressant compound. Exemplary conditions in which immunosuppression is useful include transplant rejections, in which the immunosuppressant drug delays or prevents transplant rejection. Graft versus host disease can be prevented or ameliorated by treating the graft with a compound described herein.

Autoimmune and immune related disorders and diseases can also be treated or prevented as described herein. Exemplary autoimmune diseases and immune related disorder include systemic lupus erythematosis, rheumatoid arthritis, osteoarthritis, juvenile chronic arthritis, a spondyloarthropathy, systemic sclerosis, an idiopathic inflammatory myopathy, Sjogren's syndrome, systemic vasculitis, sarcoidosis, autoimmune hemolytic anemia, autoimmune thrombocytopenia, thyroiditis, diabetes mellitus, immune-mediated renal disease, a demyelinating disease of the central or peripheral nervous system, idiopathic demyelinating polyneuropathy, Guillain-Barr syndrome, a chronic inflammatory demyelinating polyneuropathy, a hepatobiliary disease, infectious or autoimmune chronic active hepatitis, primary biliary cirrhosis, granulomatous hepatitis, sclerosing cholangitis, inflammatory bowel disease, gluten-sensitive enteropathy, Whipple's disease, an autoimmune or immune-mediated skin disease, a bullous skin disease, erythema multiforme, contact dermatitis, psoriasis, an allergic disease, asthma, allergic rhinitis, atopic dermatitis, food hypersensitivity, urticaria, an immunologic disease of the lung, eosinophilic pneumonias, idiopathic pulmonary fibrosis, hypersensitivity pneumonitis, systemic lupus erythematosus, scleroderma, and arthritis.

Administration of an modulator of Nampt expression or activity also can be used for modulating sensitization to genotoxic agents. A “genotoxic agent” or “genotoxin” refers to any chemical compound or treatment method that induces DNA damage when applied to a cell. “DNA damage”, as used herein, refers to chemical and/or physical modification of the DNA in a cell, including methylation, alkylation, double-stranded breaks, cross-linking, thymidine dimers caused by ultraviolet light, and oxidative lesions formed by oxygen radical binding to DNA bases. Genotoxic agents can be chemical or radioactive. A genotoxic agent is one for which a primary biological activity of the chemical (or a metabolite) is alteration of the information encoded in the DNA. Genotoxic agents can vary in their mechanism of action, and can include: alkylating agents such as ethylmethane sulfonate (EMS), nitrosoguanine and vinyl chloride; bulky addition products such as benzo(a)pyrene and aflatoxin B 1; reactive oxygen species such as superoxide, hydroxyl radical; base analogs such as 5-bromouracil; intercalating agents such as acridine orange and ethidium bromide. A variety of chemical compounds, also described as “chemotherapeutic agents,” function to induce DNA damage and are thus genotoxic agents as used herein. Chemotherapeutic agents include, e.g., adriamycin, 5-fluorouracil (5FU), etoposide (VP-16), camptothecin, actinomycin-D, mitomycin C, cisplatin (CDDP) and even hydrogen peroxide. Genotoxic agents also include radiation and electromagnetic waves that induce DNA damage such as gamma-irradiation, X-rays, UV-irradiation, microwaves, electronic emissions, and the like. In addition, certain chemicals, sometimes called indirect genotoxic agents, can be converted into genotoxic agents by normal metabolic enzymes. As used herein, genotoxic agents refer to both direct and indirect genotoxic agents. Genotoxic agents cause mutations in DNA, and can cause cancer. The term “genotoxic agents” also encompasses a combination of one or more DNA damaging agents, whether radiation-based or compounds.

Because of the wide diversity of genotoxic agents, exposure to genotoxic agents comes in many different forms. Mechanisms of exposure to chemical genotoxic agents may include direct contact, or inhalation or ingestion by the subject. In the case of radiation, exposure may arise from proximity to a source of ionizing radiation. The nature of exposure to these genotoxic agents can also vary. Exposure can be deliberate, as is the case with chemotherapy and radiotherapy, but may also be accidental. Examples of accidental exposure may include occupational chemical exposure in a laboratory, factory or farm, or occupational exposure to ionizing radiation in a nuclear power plant, clinic, laboratory, or by frequent airplane travel.

Another aspect provides methods for increasing the effectiveness of a therapeutic compound for treating a disease or disorder. In this aspect, agents (e.g., SIRT1 modulators) are used as adjunct therapies to improve an existing therapy. For example, where an existing therapy is more effective if administered or metabolized at certain periods of the circadian rhythm, the methods described herein can be used to favorably affect the existing therapy by regulating the circadian rhythm. The methods include administering to a subject in need of such treatment an agent that modulates SIRT1 activity or expression or that modulates binding of SIRT1 to CLOCK or CLOCK/BMAL, in an amount effective to modulate a circadian rhythm of the subject. In so doing, the effectiveness of the therapeutic compound is increased relative to the effectiveness of the therapeutic compound without the administration of the agent. The increased effectiveness of the therapeutic compound is a greater response of the subject to the same or a lesser dose of the therapeutic compound, or the same response of the subject to a lesser dose of the therapeutic compound.

In some aspects, the methods disclosed herein include first testing the subject to determine if the subject's disease or disorder has a circadian rhythm component according to methods known in the art. If so, then the agents described here can be administered to the subject.

The invention provides for treating diseases and disorders based on the recognition that SIRT1 affects CLOCK activity, which regulates circadian rhythm. Thus a variety of diseases and disorders that have a circadian rhythm component or that are directly related to disruption or alteration of circadian rhythm can be treated in accordance with the invention.

Diseases, disorders and conditions in which such methods are useful include sleep disorders; psychiatric disorder associated with circadian rhythm; mitochondrial diseases; metabolic disorders; neurologic disorders; muscular disorders; cardiovascular diseases; and excessive weight or obesity.

Sleep disorders include insomnia, jet lag, shift work sleep disorder, delayed sleep phase syndrome (DSPS), advanced sleep phase syndrome (ASPS), non 24-hour sleep wake disorder and irregular sleep-wake pattern.

Psychiatric disorders associated with circadian rhythm include depression, seasonal affective disorder, dementia and rapid-cycling bipolar disorder.

Neurological and neurodegenerative diseases with a circadian rhythm component include Alzheimer's disease.

Additional diseases and disorders with circadian components include anorexia nervosa; abnormal blood pressure; abnormal heart rate; asthma; metabolic disorders such as diabetes, abnormal insulin secretion, abnormal plasma glucose levels, obesity, and metabolic syndrome; and cancer.

Specific metabolic disorders, diseases or conditions include insulin resistance, diabetes, diabetes related conditions or disorders, or metabolic syndrome. Other metabolic disorders will be known to the skilled person.

Cardiovascular diseases that can be treated include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to the retinal arterioles, the glomerular arterioles, the vasa nervorum, cardiac arterioles, and associated capillary beds of the eye, the kidney, the heart, and the central and peripheral nervous systems.

Neurological diseases that can be treated include neurodegenerative diseases. Some non-limiting examples of neurodegenerative disorders include stroke, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, Multiple Sclerosis (MS), and Friedreich's ataxia, Periventricular leukomalacia (PVL), ALS-Parkinson's-Dementia complex of Guam, Wilson's disease, cerebral palsy, progressive supranuclear palsy (Steel-Richardson syndrome), bulbar and pseudobulbar palsy, diabetic retinopathy, multi-infarct dementia, macular degeneration, Pick's disease, diffuse Lewy body disease, prion diseases such as Creutzfeldt-Jakob, Gerstmann-Straussler-Scheinker disease, Kuru and fatal familial insomnia, primary lateral sclerosis, degenerative ataxias, Machado-Joseph disease/spinocerebellar ataxia type 3 and olivopontocerebellar degenerations, spinal and spinobulbar muscular atrophy (Kennedy's disease), familial spastic paraplegia, Wohlfart-Kugelberg-Welander disease, Tay-Sach's disease, multisystem degeneration (Shy-Drager syndrome), Gilles De La Tourette's disease, familial dysautonomia (Riley-Day syndrome), Kugelberg-Welander disease, subacute sclerosing panencephalitis, Werdnig-Hoffmann disease, synucleinopathies (including multiple system atrophy), Sandhoff disease, cortical basal degeneration, spastic paraparesis, primary progressive aphasia, progressive multifocal leukoencephalopathy, striatonigral degeneration, familial spastic disease, chronic epileptic conditions associated with neurodegeneration, Binswanger's disease, and dementia (including all underlying etiologies of dementia). Muscular diseases, including neuromuscular diseases, that can be treated include: muscular dystrophy and myopathy.

Mitochondrial diseases that can be treated include diseases that show a variety of symptoms caused by dysfunction of mitochondria in cells. The mitochondrial disease are classified in various ways by biochemical abnormalities, clinical symptoms or types of DNA abnormalities. Types named as KSS (chronic progressive external ophthalmoplegia), MERRF (myoclonus epilepsy associated with ragged-red fibers; Fukuhara syndrome), MELAS, Leber's disease, Leigh encephalopathia and Pearson's disease are widely known. Among them, MELAS is a type mainly showing stroke-like episodes, occupies 30% or more of the whole and is believed to be the most frequent type in the mitochondrial disease.

Insulin resistance disorders that may be treated include any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

The methods of the invention include in some embodiments administering, to a subject in need of such treatment, an agent that modulates (i.e., increases or decreases) the protein or activity level of SIRT1 in cells of the subject. A variety of administration methods are known in the art and can be used in the methods described herein. In some embodiments, the agent optionally is targeted to, or administered into, a cell of the subject.

As used herein, the terms “increase SIRT1”, “activate SIRT1” and the like mean that the activity of SIRT1 is increased. The activity of SIRT1 can be increased by increasing the activity of the SIRT1 polypeptide and/or by increasing the amount of active SIRT1polypeptide. Likewise, as used herein, the terms “decrease SIRT1”, “inhibit SIRT1” and the like mean that the activity of SIRT1 is decreased. The activity of SIRT1 can be decreased by decreasing the activity of the SIRT1 polypeptide and/or by decreasing the amount of active SIRT1 polypeptide. Molecules that increase or decrease SIRT1 activity are generically referred to as “SIRT1 modulators”. SIRT1 modulators include SIRT1 activators and SIRT1 inhibitors, any of which may also be referred to herein as “pharmacological agents”, “active compounds”, “components”, “therapeutics” and the like.

In some embodiments the activity or protein level of a sirtuin such as SIRT1 is increased through administering the SIRT1 gene or protein. In some embodiments the activity or protein level of a sirtuin such as SIRT1 is increased through administering a compound that increases the protein level or increases the activity a sirtuin. In some embodiments, SIRT1 activators may be any SIRT1 activator that is known in the art. SIRT1 activators are described in numerous U.S. application publications, PCT publications, and references, all of which are specifically incorporated by reference herein. Methods for activating sirtuins, and non-limiting examples of SIRT1 activators and inhibitors include compounds described in: US 2009/0012080, US 2008/0194803, US 2008/0255382, US 2007/0149466, US 2007/0117765, US 2007/0043050, US 2007/0037865, US 2007/0037827, US 2007/0037810, US 2007/0037809, US 2007/0014833, US 2006/0276416, US 2006/0276393, US 2006/0229265, US 2006/0084085, US 2006/0025337, US 2005/0267023, US 2005/0171027, US 2005/0136537, US 2005/0096256, WO 05/002555, WO 2005/065667, WO 2007/084162 and in U.S. Pat. No. 7,345,178, all of which are incorporated by reference herein in their entirety, in particular for these teachings.

SIRT1 activators also include SRT 501, a formulation of resveratrol with greater bioavailability(Sirtris), and SRT1460 (Sirtris).

SIRT1 inhibitors also include RNA inhibitory molecules (RNAi) as described in US 2007/0185049 which is hereby incorporated by reference in its entirety and as described elsewhere herein. Sirtuin inhibitors also include those disclosed in Grozinger et al., J. Biol. Chem. 42:38837-43 (2001), which is hereby incorporated by reference in its entirety. Methods for measurement of activation of SIRT1 are well known in the art, including in the above-referenced patents and patent publications.

SIRT1 modulators may be administered by any of the known methods, e.g., systemically or locally, topically, intradermally, subcutaneously, intramuscularly, or orally.

Also provided are methods for modulating CLOCK acetylase activity or BMAL acetylation in a eukaryotic cell, preferably a human cell. The methods include modulating the expression or activity of SIRT1. Modulation of expression or activity of SIRT1 includes increasing or decreasing SIRT1 expression or activity. Increasing SIRT1 expression can be produced by expressing exogenous SIRT1 in the cell or by increasing expression of endogenous SIRT1. Decreasing SIRT1 expression can be produced by contacting the cell with a molecule that interferes with SIRT1 expression, such as a siRNA molecule. Increasing SIRT1 activity can be produced by contacting the cell with one or more SIRT1 activators, which are described elsewhere herein. Decreasing SIRT1 activity can be produced by contacting the cell with one or more SIRT1 inhibitors, which are described elsewhere herein. Modulation of BMAL acetylation includes reducing acetylation (increasing deacetylation) of lysine 537 of BMAL.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting.

The methods described herein may be applied in vitro or in vivo. For example, they may be applied to cells in vitro, either cells from cell lines or cells obtained from a subject.

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

A “form that is naturally occurring”, when referring to a compound, means a compound that is in a form, e.g., a composition, in which it can be found naturally. For example, since resveratrol can be found in red wine, it is present in red wine in a form that is naturally occurring. A compound is not in a form that is naturally occurring if, e.g., the compound has been purified and separated from at least some of the other molecules that are found with the compound in nature. A “naturally occurring compound” refers to a compound that can be found in nature, i.e., a compound that has not been designed by man. A naturally occurring compound may have been made by man or by nature. A “non-naturally occurring compound” is a compound that is not known to exist in nature or that does not occur in nature.

“Sirtuin modulator” refers to a compound that up regulates (e.g., activate or stimulate), down regulates (e.g., inhibit or suppress) or otherwise changes a functional property or biological activity of a sirtuin protein. Sirtuin modulators may act to modulate a sirtuin protein either directly or indirectly. In certain embodiments, a sirtuin modulator may be a sirtuin activator or a sirtuin inhibitor.

The terms “SIRT1 activator” or “SIRT1 activating compound” and the like refers to a compound that increases the level of a sirtuin protein and/or increases at least one activity of a sirtuin protein. In an exemplary embodiment, a sirtuin activator may increase at least one biological activity of a sirtuin protein by at least about 10%, 25%, 50%, 75%, 100%, or more.

“Inhibiting a sirtuin protein” refers to the action of reducing at least one of the biological activities of a sirtuin protein to at least some extent, e.g., at least about 10%, 50%, 2 fold or more.

An “inhibitory compound” or “inhibiting compound” or “sirtuin inhibitor” or “SIRT1 activating compound” and the like refers to a compound that inhibits a sirtuin protein, particularly SIRT1. “Sirtuin inhibitor” refers to a compound that decreases the level of a sirtuin protein and/or decreases at least one activity of a sirtuin protein. In an exemplary embodiment, a sirtuin inhibitor may decrease at least one biological activity of a sirtuin protein by at least about 10%, 25%, 50%, 75%, 100%, or more.

A “direct activator” of a sirtuin is a molecule that activates a sirtuin by binding to it. A “direct inhibitor” of a sirtuin is a molecule that inhibits a sirtuin by binding to it.

SIRT1 activators and inhibitors include compounds described in: US 2009/0012080, US 2008/0194803, US 2008/0255382, US 2007/0149466, US 2007/0117765, US 2007/0043050, US 2007/0037865, US 2007/0037827, US 2007/0037810, US 2007/0037809, US 2007/0014833, US 2006/0276416, US 2006/0276393, US 2006/0229265, US 2006/0084085, US 2006/0025337, US 2005/0267023, US 2005/0171027, US 2005/0136537, US 2005/0096256, WO 05/002555, WO 2005/065667, WO 2007/084162 and in U.S. Pat. No. 7,345,178, all of which are incorporated by reference herein in their entirety, in particular for these teachings.

SIRT1 activators also include SRT501 (Sirtris), a formulation of resveratrol with greater bioavailability, SRT1720 (Sirtris) (Milne et al., Nature 450: 712-716, 2007), SRT2183 (Sirtris) (Milne et al., Nature 450: 712-716, 2007) and SRT1460 (Sirtris) (Milne et al., Nature 450: 712-716, 2007).

SRT1720 is N-(2-(3-(piperazin-1-ylmethyl)imidazo[2,1-b]thiazol-6-yl)phenyl)quinoxaline-2-carboxamide; the structure of SRT1720 is:

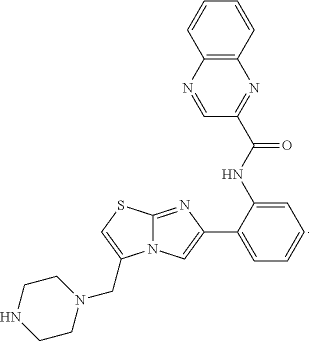

SRT2183 is (R)—N-(2-(3-((3-hydroxypyrrolindin-1-yl)methyl)imidazo[2,1-b]thiazol-6-yl)phenyl)-2-naphthamide; The structure of SRT2183 is:

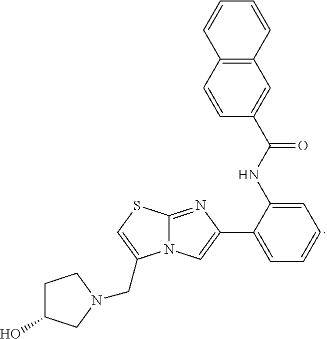

SRT1460 is 3,4,5-trimethoxy-N-(2-(3-(piperazin-1-ylmethyl)imidazo[2,1-b]thiazol-6-yl)phenyl)benzamide; the structure of SRT1460 is:

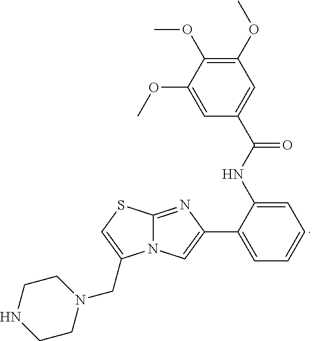

Examples of sirtuin modulators described in U.S. Pat. No. 7,345,178 include the following. In one embodiment, sirtuin-modulating compounds are represented by Structural Formula (I):

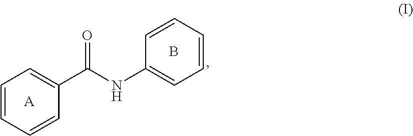

or a salt thereof, where:

Ring A is optionally substituted; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In certain embodiments, Ring B is substituted with at least a carboxy group.

In certain embodiments, Ring B is substituted with at least a substituted or unsubstituted arylcarboxamine, a substituted or unsubstituted aralkylcarboxamine or a polycyclic aryl group.

In certain embodiments, Ring B is substituted with at least a substituted or unsubstituted heteroaryl group or a substituted or unsubstituted heterocyclylcarbonylethenyl group.

In another embodiment, sirtuin-modulating compounds are represented by Structural Formula (II):

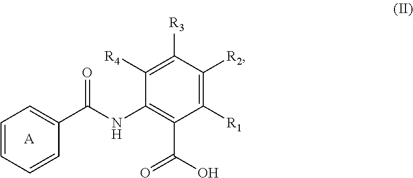

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In a further embodiment, sirtuin-modulating compounds are represented by Structural Formula (IIa):

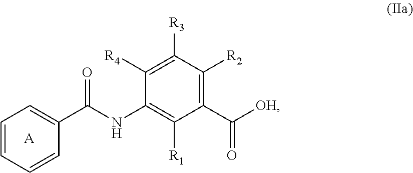

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another embodiment, sirtuin-modulating compounds are represented by Structural Formula (II):

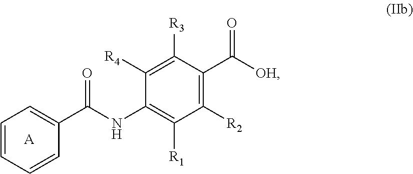

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In certain embodiments, R₁, R₂, R₃ and R₄ in Structural Formulas (II)-(IIb) are independently selected from the group consisting of —H, —OR₅ and —SR₅, particularly —H and —OR₅ (e.g., —H, —OH, —OCH₃).

Ring A is preferably substituted. Suitable substituents include halogens (e.g., bromine), acyloxy groups (e.g., acetoxy), aminocarbonyl groups (e.g., arylaminocarbonyl such as substituted, particularly carboxy-substituted, phenylaminocarbonyl groups) and alkoxy (e.g., methoxy, ethoxy) groups.

In yet another aspect, the invention utilizes sirtuin-modulating compounds of Formula (III):

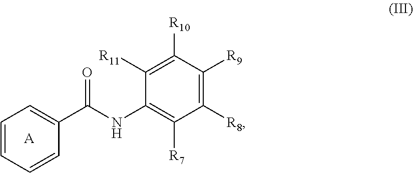

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₈ is a polycyclic aryl group; and

n is 1 or 2.

In certain embodiments, one or more of R₇, R₉, R₁₀ and R₁₁ are —H. In particular embodiments, R₇, R₉, R₁₀ and R₁₁ are each —H.

In certain embodiments, R₈ is a heteroaryl group, such as an oxazolo[4,5-b]pyridyl group. In particular embodiments, R₈ is a heteroaryl group and one or more of R₇, R₉, R₁₀ and R₁₁ are —H.

Ring A is preferably substituted. Suitable substituents include halogens (e.g., bromine), acyloxy groups (e.g., acetoxy), aminocarbonyl groups (e.g., arylaminocarbonyl, such as substituted, particularly carboxy-substituted, phenylaminocarbonyl groups) and alkoxy (e.g., methoxy, ethoxy) groups, particularly alkoxy groups. In certain embodiments, Ring A is substituted with at least one alkoxy or halo group, particularly methoxy.

In certain embodiments, Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle.

In certain embodiments, Ring A is not substituted with a nitrile or pyrrolidyl group.

In certain embodiments, R₈ is a substituted or unsubstituted bicyclic heteroaryl group, such as a bicyclic heteroaryl group that includes a ring N atom and 1 to 2 additional ring heteroatoms independently selected from N, O or S. Preferably, R₈ is attached to the remainder of the compound by a carbon-carbon bond. In certain such embodiments, 2 additional ring heteroatoms are present, and typically at least one of said additional ring heteroatoms is O or S. In certain such embodiments, 2 total ring nitrogen atoms are present (with zero or one O or S present), and the nitrogen atoms are typically each in a different ring. In certain such embodiments, R₈ is not substituted with a carbonyl-containing moiety, particularly when R₈ is thienopyrimidyl or thienopyridinyl.

In certain such embodiments, R₈ is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In certain such embodiments, R₈ is selected from thiazolopyridyl, imidazothiazolyl, benzoxazinonyl, or imidazopyridyl.

Particular examples of R₈, where [symbol] indicates attachment to the remainder of Structural Formula (III), include:

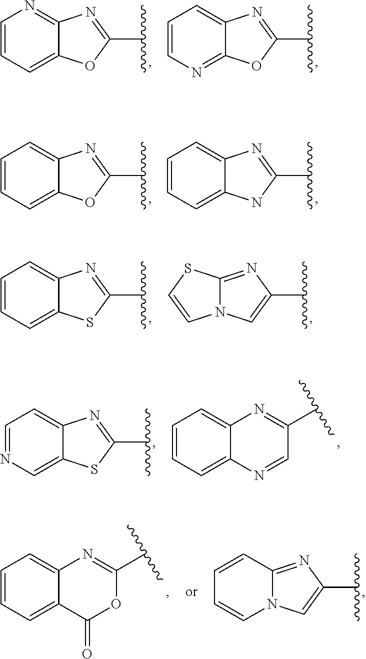

where up to 2 ring carbons not immediately adjacent to the indicated attachment point are independently substituted with O—C₁-C₃ straight or branched alkyl, C₁-C₃ straight or branched alkyl or halo, particularly C₁-C₃ straight or branched alkyl or halo. In certain embodiments, R₈ is

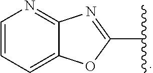

In certain embodiments (e.g., when the modulator is a sirtuin activator), R₈ is

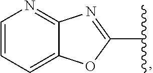

and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle. In certain such embodiments, Ring A is not simultaneously substituted at the 2- and 6-positions with O—(C₁-C₃ straight or branched alkyl). In certain such embodiments, Ring A is not simultaneously substituted at the 2-, 4- and 6-positions with O—(C₁-C₃ straight or branched alkyl). In certain such embodiments, Ring A is not simultaneously substituted at the 2-, 3-, and 4-positions with O—(C₁-C₃ straight or branched alkyl). In certain such embodiments, Ring A is not substituted at the 4-position with a 5 to 6-membered heterocycle. In certain such embodiments, Ring A is not singly substituted at the 3- or 4-position (typically 4-position) with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

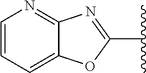

and Ring A is optionally substituted with up to 3 substituents independently selected from (C₁-C₃ straight or branched alkyl), (C₁-C₃ straight or branched haloalkyl, where a haloalkyl group is an alkyl group substituted with one or more halogen atoms), O—(C₁-C₃ straight or branched alkyl), N(C₁-C₃ straight or branched alkyl)₂, halo, or a 5 to 6-membered heterocycle. In certain such embodiments, Ring A is not singly substituted at the 3- or 4-position with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

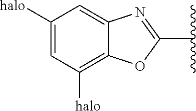

(e.g., where one or both halo is chlorine) and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle, but not singly substituted at the 3-position with O—(C₁-C₃ straight or branched alkyl).

In certain embodiments, such as when R₈ has one of the values described above, Ring A is substituted with up to 3 substituents independently selected from chloro, methyl, O-methyl, N(CH₃)₂ or morpholino. In certain such embodiments, R₈ is selected from

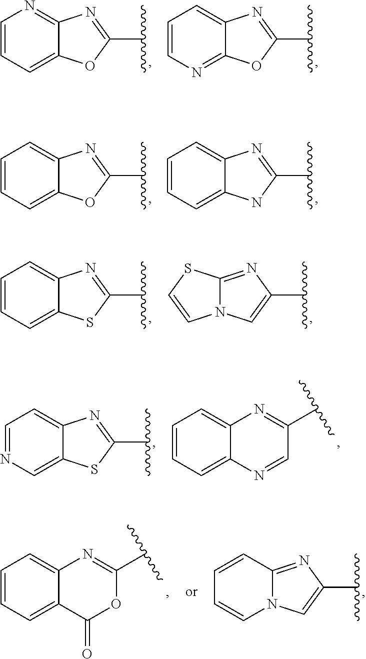

where up to 2 ring carbons not immediately adjacent to the indicated attachment point are independently substituted with C₁-C₃ straight or branched alkyl or halo; each of R₇, R₉, and R₁₁ is —H; and R₁₀ is selected from —H, —CH₂OH, —CO₂H, —CO₂CH₃, —CH₂-piperazinyl, CH₂N(CH₃)₂, —C(O)—NH—(CH₂)₂—N(CH₃)₂, or —C(O)-piperazinyl. In certain such embodiments, when R₈ is

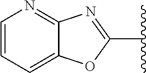

and Ring A is 3-dimethylaminophenyl, none of R₇, R₉, R₁₀ and R₁₁ is —CH₂—N(CH₃)₂ or —C(O)—NH—(CH₂)₂—N(CH₃)₂, and/or when R₈ is

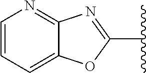

and Ring A is 3,4 dimethoxyphenyl, none of R₇, R₉, R₁₀ and R₁₁ is C(O)OCH₃ or C(O)OH.

In certain embodiments, such as when R₈ has one of the values described above and/or Ring A is optionally substituted as described above, at least one of R₇, R₉, R₁₀ and R₁₁ is —H. In certain such embodiments, each of R₇, R₉, R₁₀ and R₁₁ is —H.

In certain embodiments, R₇, R₉, R₁₀ or R₁₁ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2. In certain such embodiments, R₁₀ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙpyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2, and each of R₇, R₉, and R₁₁ is H.

In certain embodiments, Ring A is substituted with a nitrile group or is substituted at the para position with a 5- or 6-membered heterocycle. Typical examples of the heterocycle include pyrrolidyl, piperidinyl and morpholinyl.

In yet another aspect, the invention utilizes sirtuin-modulating compounds of Formula (IV):

Ar-L-J-M-K—Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, SR₁₀′, NR₁₀′R₁₀′, COOR₁₀′, NO₂, CN, C(O)R₁₀′, C(O)NR₁₀′R₁₀′, NHC(O)R₁₀, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a preferred embodiment of the invention, each Ar, L, and Ar′ is independently an optionally substituted 5- to 7-membered monocyclic ring system or an optionally substituted 9- to 12-membered bicyclic ring system.

According to another preferred embodiment,
Ar is

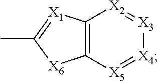

X₁, X₂, X₃, X₄, and X₅ are independently selected from CR₁′ and N; and
X₆ is selected from NR₁′, O, and S;

According to yet another preferred embodiment, X₁ and X₂ are N; X₃, X₄, and X₅ are CR₁′; and X₆ is O.

According to still yet another preferred embodiment, X₁ and X₃ are N; X₂, X₄, and X₅ are CR₁′; and X₆ is O.

According to still yet another preferred embodiment, X₁ and X₄ are N; X₂, X₃, and X₅ are CR₁′; and X₆ is O.

According to still yet another preferred embodiment, X₁ and X₅ are N; X₂, X₃, and X₄ are CR₁′; and X₆ is O.

In another embodiment, the compounds of the formula above are those wherein J is NR₁′, K is absent, and M is C(O).

In yet another embodiment, the compounds of the formula above are those wherein J is absent, K is NR₁′, and M is C(O).

In a further embodiment, compounds of formula (IV) are those where when J is absent and K is NR₁′, M is not C(O) and when J is NR₁′ and K is absent, M is not C(O).

In a preferred embodiment, the compounds above are those wherein L is an optionally substituted 5- to 7-membered carbocyclic or heterocyclic aryl group.

In yet another preferred embodiment, the compounds are those wherein L is an optionally substituted phenylene, pyridinylene, imidazolylene, oxazolylene, or thiazolylene.

In a particularly preferred embodiment, L is an optionally substituted phenylene.

In another particularly preferred embodiment, L is an optionally substituted pyridinylene.

In an even more preferred embodiment, L is phenylene.

In another even more preferred embodiment, L is pyridinylene.

In either of these embodiments, Ar and J may be attached to L at the ortho-, meta-, or para-positions. Particularly preferred are those embodiments where attachment is at the meta-position.

In certain embodiments, L is not phenylene when Ar′ is phenyl. Examples of such embodiments include embodiments where L is an optionally substituted heterocyclic aryl group and Ar′ is an optionally substituted carbocyclic or heterocyclic aryl group, or wherein L is an optionally substituted carbocyclic or heterocyclic aryl group and Ar′ is an optionally substituted heterocyclic aryl group.

In yet another aspect, the invention provides novel sirtuin-modulating compounds of Formula (I) or a salt thereof, wherein

Ring A is substituted with at least one R₁′ group;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group;

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′; and

Ring B is substituted with at least one

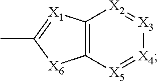

wherein

X₁, X₂, X₃, X₄, and X₅ are independently selected from CR₁′ and N; and

X₆ is selected from NR₁′, O, and S.

In a preferred embodiment, Ring B is phenyl or pyridinyl.

In a further aspect, the invention utilizes novel sirtuin-modulating compounds of Formula (IVa):

Het-L-Q-Ar′ (IVa)

or a salt thereof, wherein:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R′₁—NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—S(O)₂—NR₁′—, —NR₁′—CR₁′R′₁—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—

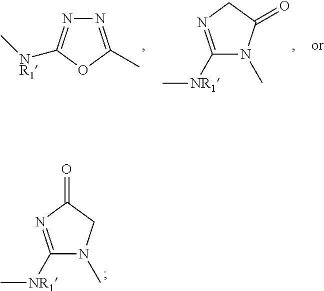

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In certain embodiments, when Het is a polycyclic heteroaryl, L is optionally substituted phenylene, and Ar′ is optionally substituted phenyl; then Q is not —NH—C(O)—.

In certain embodiments (e.g., when the compound is a sirtuin activator), Het and Q are attached to L in a 1-, 2- or 1-,3-configuration (e.g., when L is phenylene, Het and Q are attached in an ortho or a meta orientation). In certain embodiments where Het and Q are attached to L in a 1-,3-configuration, if Het is benzoxazolyl, L is pyridylene and Q is —NH—C(O)—NH, then Ar′ is not 3,4 dioxymethlyene phenyl; if Het is methyl thiazolyl, L is phenylene and Q is —NH—C(O)—, then Ar′ is not 3-dimethylamino phenyl; if Het is oxazolopyridyl, L is pyridylene and Q is —NH—C(O)—NH, then Ar′ is not 4-dimethylamino phenyl; if Het is oxazolopyridyl or benzoxazolyl and L is

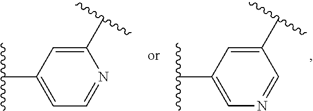

then Q is not —NH—(SO)₂—; and if Het is oxazolopyridyl, L is

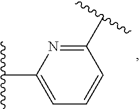

and Q is —NH—C(O)—, then Ar′ is not 3,4 dimethoxyphenyl or pyridyl.

When Het is substituted, it is typically substituted at up to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

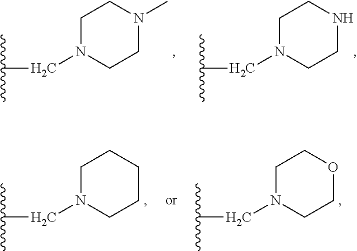

where each R₁₂ is independently selected from optionally substituted C₁-C₃ straight or branched alkyl.

In certain embodiments, Het is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In other embodiments, Het comprises one ring N heteroatom and 1 to 2 additional ring heteroatoms independently selected from N, O or S, such as thiazolyl, triazolyl, oxadiazolyl, thiazolopyridyl, imidazothiazolyl, benzoxazinonyl, or imidazopyridyl.

Particular examples of Het include:

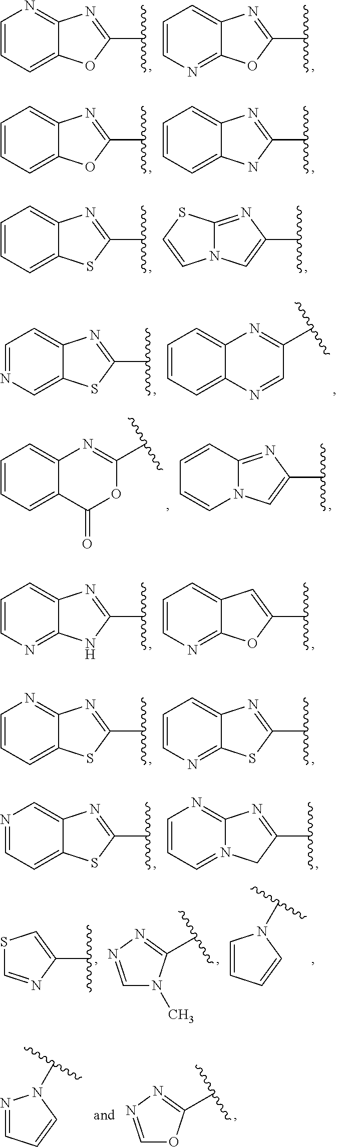

where up to 2 ring carbons not immediately adjacent to the indicated attachment point are independently substituted with optionally substituted C₁-C₃ straight or branched alkyl, phenyl, halo, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

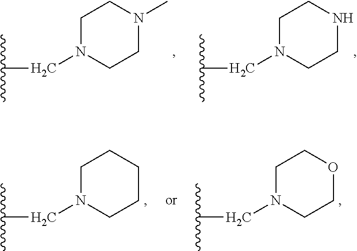

wherein each R₁₂ is independently selected from optionally substituted C₁-C₃ straight or branched alkyl.

In certain embodiments, L is selected from

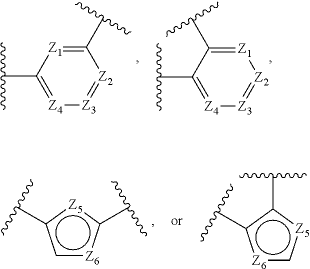

wherein:

each of Z₁, Z₂, Z₃ and Z₄ is independently selected from CH or N, wherein not more than three of said Z₁, Z₂, Z₃ or Z₄ is N;

each of Z₅ and Z₆ is independently selected from C, N, O or S, provided that at least one of Z₅ and Z₆ is N; and

L is optionally substituted at 1 to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

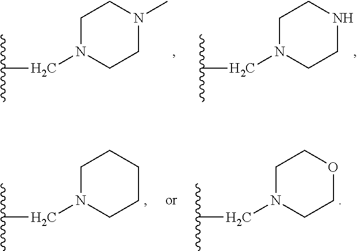

In preferred embodiments, L is selected from phenylene or pyridylene, such as unsubstituted phenylene or phenylene substituted with a single substituent selected from C(O)OCH₃, C(O)OH, CH₂OH, N(CH₃)₂, or CH₂N(CH₃)₂, or unsubstituted pyridylene.

In certain embodiments, Q is selected from —NH—C(O)—, —NH—S(O)₂—, —NH—C(O)—NH—, —C(O)—NH—, —CH₂—, —N(CH₃)—C(O)—NH—, —NH—C(O)—N(CH₃)—, or —NH—S(O)₂—NH—, particularly —NH—C(O)—, —C(O)—NH—, —NH—, —NH—C(O)—NH, or —NH—S(O)₂—.

In certain embodiments, Ar′ is selected from optionally substituted phenyl, benzothiazolyl, or benzoxazolyl. When Ar′ is phenyl, typical optional substituents are 1 to 3 substituents independently selected from halo, (optionally substituted C₁-C₃ straight or branched alkyl), O-(optionally substituted C₁-C₃ straight or branched alkyl), S-(optionally substituted C₁-C₃ straight or branched alkyl), N(CH₃)₂ or optionally substituted heterocyclyl, or wherein two substituents on adjacent ring atoms are taken together to form a dioxymethylene.

In certain embodiments, Het is selected from

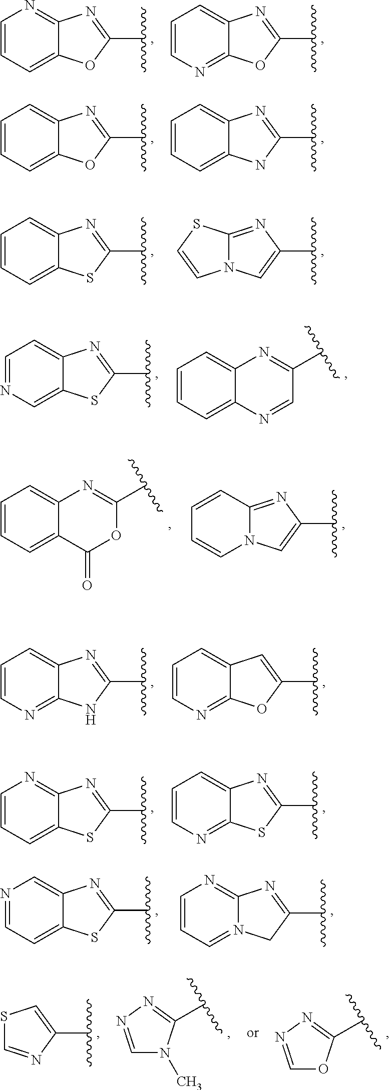

and wherein up to 2 ring carbons not immediately adjacent to the indicated attachment point are independently substituted with optionally substituted C₁-C₃ straight or branched alkyl, phenyl or halo;

L is selected from unsubstituted phenylene, phenylene substituted with a single substituent selected from C(O)OCH₃, C(O)OH, CH₂OH, N(CH₃)₂, or CH₂N(CH₃)₂, or unsubstituted pyridylene;

Q is selected from —NH—C(O)—, —C(O)—NH—, —NH—, —NH—C(O)—NH, or —NH—S(O)₂—; and

Ar′ is selected from optionally substituted phenyl, benzothiazolyl, or benzoxazolyl, wherein said phenyl is optionally substituted with 1 to 3 substituents independently selected from chloro, methyl, O-methyl, S-methyl, N(CH₃)₂, morpholino, or 3,4 dioxymethylene.

In certain embodiments, Q is selected from —NH—C(O)—, —C(O)—NH—, —NH— or —NH—C(O)—NH.

In certain embodiments, the substituents on Ar′ are selected from chloro, methyl, O-methyl, S-methyl or N(CH₃)₂. In certain embodiments, the only substituent on Ar′ is an O-methyl group, particularly an O-methyl group ortho or meta to Q. In certain embodiments, when there are two or more O-methyl groups or Ar′, at least one is ortho or meta to Q.

In certain embodiments, L is pyridyl and Het and Q are at the 1,3- or 2,4-position with respect to the pyridyl nitrogen atom. In certain such embodiments, Q is —NH—S(O)₂—.

In certain embodiments where L is further substituted, the substituent is typically meta to both Het and Q.

In certain embodiments, Q is —NH— and Het is thiazolyl or oxazolopyridyl.

In certain embodiments, Q is —NH— and Ar is benzothiazolyl or benzoxazolyl.

In certain embodiments, such as when the sirtuin modulator is a sirtuin activator, L is

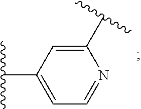

and Q is —NH—(SO)₂—. In certain such embodiments, Het is oxazolopyridyl. When L, Q and optionally Het have these values, Ar′ is advantageously naphthyl or phenyl, where Ar′ is optionally substituted with 1 to 3 substituents independently selected from CN, halo, (C₁-C₃ straight or branched alkyl), O—(C₁-C₃ straight or branched alkyl), N(C₁-C₃ straight or branched alkyl)₂, or a 5 to 6-membered heterocycle.

In certain embodiments, such as when the sirtuin modulator is a sirtuin activator, L is

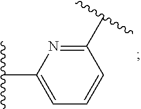

and Q is —NH—C(O)—. In certain such embodiments, Het is oxazolopyridyl. When L, Q and optionally Het have these values, Ar′ is advantageously pyridyl or phenyl optionally substituted with 1 to 3 substituents independently selected from CN, halo, (C1-C3 straight or branched alkyl), O—(C1-C3 straight or branched alkyl), N(C1-C3 straight or branched alkyl)2, or a 5 to 6-membered heterocycle.

In certain embodiments, such as when the sirtuin modulatory is a sirtuin inhibitor, Het comprises one N heteroatom and 1 to 2 additional heteroatoms independently selected from N, O or S;

L is

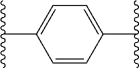

and is optionally substituted;

Q is —NH—C(O)—; and

Ar′ is phenyl substituted with 1 to 3 substituents independently selected from CN, halo, C₁-C₃ straight or branched alkyl, O—(C₁-C₃ straight or branched alkyl), N(C₁-C₃ straight or branched alkyl)₂, or a 5 to 6-membered heterocycle,

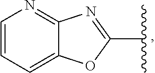

wherein when R₈ is unsubstituted then ring A is:
a) not simultaneously substituted at the 2- and 6-positions with O—(C₁-C₃ straight or branched alkyl);
b) not simultaneously substituted at the 2-position with C₁-C₃ straight or branched alkyl or O—(C₁-C₃ straight or branched alkyl) and at the 3-position with O—(C₁-C₃ straight or branched alkyl);
c) not substituted at the 4-position with O—(C₁-C₃ straight or branched alkyl) unless simultaneously substituted at the 3-position with halo or O—(C₁-C₃ straight or branched alkyl) and unsubstituted at all other positions;

not substituted at the 4-position with N(C₁-C₃ straight or branched alkyl)₂, or said 5 to 6-membered heterocycle. In certain such embodiments, L is unsubstituted and/or Het is oxazolopyridyl.

In yet another aspect, the invention utilizes novel sirtuin-modulating compounds of Formula (V):

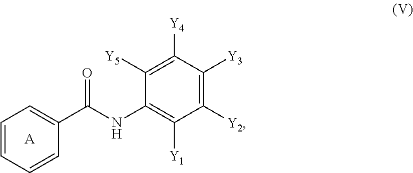

or a salt thereof, wherein:

Ring A is optionally substituted with at least one R₁′ group;

Y₁, Y₂, Y₃, Y₄, and Y₅ are independently R₁′;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉), S(O)₂NR₉′R₉′, OC(O)R₉′; or R₉′.

In a preferred embodiment of the above compound,

either Y₂ or Y₃ is

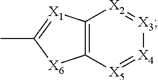

X₁, X₂, X₃, X₄, and X₅ are independently selected from CR₁′ and N; and

X₆ is selected from NR₁′, O, and S.

According to an even more preferred embodiment, X₁ and X₂ are N; X₃, X₄, and X₅ are CR₁′; and X₆ is O.

According to another even more preferred embodiment, X₁ and X₃ are N; X₂, X₄, and X₅ are CR₁′; and X₆ is O.

According to another even more preferred embodiment, X₁ and X₄ are N; X₂, X₃, and X₅ are CR₁′; and X₆ is O.

According to another even more preferred embodiment, X₁ and X₅ are N; X₂, X₃, and X₄ are CR₁′; and X₆ is O.

In another aspect, the invention provides sirtuin-modulating compounds of Structural Formula (VII):

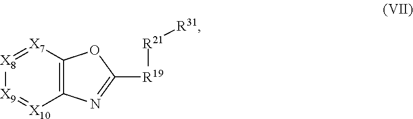

or a salt thereof, wherein:

each of X₇, X₈, X₉ and X₁₀ is independently selected from N, CR²⁰, or CR₁′, wherein:

each R²⁰ is independently selected from H or a solubilizing group;
each R₁′ is independently selected from H or optionally substituted C₁-C₃ straight or branched alkyl;

one of X₇, X₈, X₉ and X₁₀ is N and the others are selected from CR²⁰ or CR₁′; and
zero to one R²⁰ is a solubilizing group;

R¹⁹ is selected from:

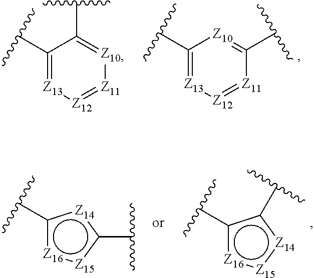

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that said compound is not:

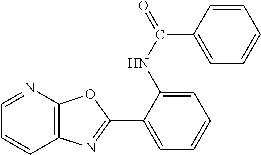

or

that when R¹⁹ is

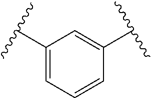

and R²¹ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

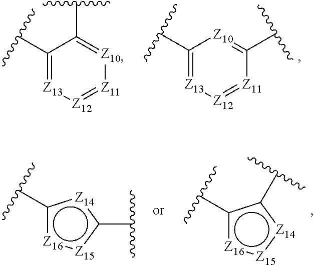

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

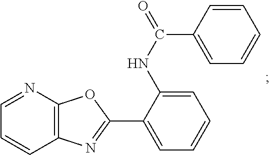

and

when X₈ and X₉ are each independently selected from CR²⁰ or CR₁′, R¹⁹ is

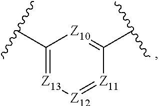

and each of Z₁₀, Z₁₁, Z₁₂ and Z₁₃ is independently selected from CR²⁰, or CR₁′, then:

a) at least one of X₈ and X₉ is not CH; or

b) at least one of Z₁₀, Z₁₁, Z₁₂ and Z₁₃ is CR²⁰, wherein R²⁰ is a solubilizing group.

In certain embodiments, when Z₁₂ is CR²⁰ and R²⁰ is a solubilizing group, the solubilizing group is not —C(O)OCH₂CH₃, —COOH,

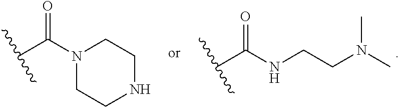

In certain embodiments, when X₈ and X₉ are each independently selected from CR²⁰ or CR₁′, R¹⁹ is

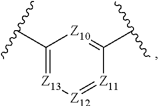

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁ and Z₁₃ is CR²⁰, wherein R²⁰ is a solubilizing group.

In certain embodiments, when R¹⁹ is

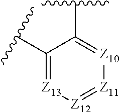

and each of Z₁₀, Z₁₁, Z₁₂ and Z₁₃ is CR²⁰, or CR₁′; X₈ and X₉ are CR²⁰ or CR₁′; R²¹ is —NHC(O)—; and R³¹ is optionally substituted phenyl, then R³¹ is a substituted phenyl, at least one R₁′ in a CR₁′ moiety is optionally substituted C₁-C₃ straight or branched alkyl, or at least one R²⁰ in a CR²⁰ is a solubilizing group, or a combination thereof.

In certain embodiments, R¹⁹ is selected from phenyl, pyridyl, thienyl or furyl.

In certain embodiments, R¹⁹ is

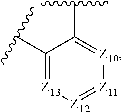

wherein each of Z₁₀, Z₁₁, Z₁₂ and Z₁₃ is independently selected from CR²⁰ or CR₁′; and

R²¹ is —NH—C(O)—; and

R³¹ is a substituted phenyl.

In certain such embodiments, when X₉ is N, R³¹ is not 2,4 dimethoxyphenyl and/or when X₁₀ is N, R³¹ is not halo substituted phenyl; 3,4-dioxoethylenephenyl; or 3,5-dimethoxyphenyl.

In preferred embodiments, R³¹ is optionally substituted with 1 to 3 substituents independently selected from —OCH₃, —CH₃, —N(CH₃)₂, pyrazinoxy or a solubilizing group. Suitable examples of R³¹ include 3-methoxy-4-((4-methylpiperazin-1-yl)methyl)phenyl, 3-methoxy-4-morpholinomethylphenyl, 3-methoxy-4-diaminomethylphenyl, 3-methoxy-4-((pyrrolidin-1-yl)methyl)phenyl, 3,4-dimethoxyphenyl, 3,5-dimethoxyphenyl, 2,3,4-trimethoxyphenyl, 3,4,5-trimethoxyphenyl, 2-dimethylaminophenyl, 3-dimethylaminophenyl, 4-dimethylaminophenyl, or 3,5-dimethylphenyl.

In certain embodiments, R¹⁹ is

selected from

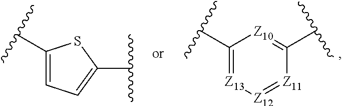

wherein one of Z₁₀, Z₁₁, Z₁₂, and Z₁₃ is N and the others are independently selected from CR²⁰ or CR₁′;

R²¹ is selected from —NH—, —NH—C(O)—, —NH—C(O)—NH, —NH—C(S)—NH— or —NH—S(O)₂—; and

R³¹ is selected from an optionally substituted phenyl, an optionally substituted naphthyl, or an optionally substituted heteroaryl.

In certain such embodiments,
a) when R²¹ is —NH—S(O)₂—, either:
   i) Z₁₀ is N; or
   ii) Z₁₁ is N and R³¹ is halophenyl or 2-methoxy-5-methylphenyl;
b) when R¹⁹ is

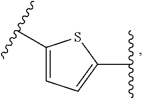

R³¹ is not 4-dimethylaminophenyl, 2,3,4-trimethoxyphenyl, or 3,5 dimethoxyphenyl; and/or
c) when R²¹ is —NH—C(O)—NH— and Z₁₀ is N, R³¹ is not 4-dimethylaminophenyl.

In certain such embodiments, R³¹ is selected from optionally substituted phenyl, benzothiazolyl, or benzoxazolyl.

In yet another embodiment, the invention utilizes sirtuin-modulating compounds of Structural Formula (VIII):

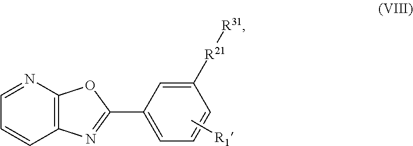

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁₁—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

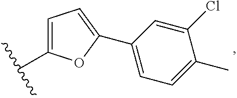

1-methoxynaphthyl, 2-methoxynaphthyl, or unsubstituted 2-thienyl;

when R₁′ is methyl, and R²¹ is —NH—C(O)—CH═CH—, R³¹ is not

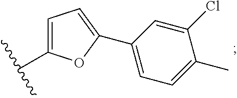

when R₁′ is methyl, and R²¹ is —NH—C(O)—CH—O—, R³¹ is not unsubstituted naphthyl, 2-methoxy, 4-nitrophenyl, 4-chloro-2-methylphenyl, or 4-t-butylphenyl; and

when R²¹ is —NH—C(O)—, R³¹ is not optionally substituted phenyl.

In certain embodiments, R²¹ is —NH—C(O)—; and R³¹ is phenyl optionally substituted with 1 to 3 substituents independently selected from —OCH₃, —CH₃, —N(CH₃)₂, or a solubilizing group.

In certain such embodiments, R²¹ is —NH—C(O)— and R³¹ is selected from unsubstituted phenyl, 2-methoxyphenyl, 3-methoxyphenyl, 2,3,4-trimethoxyphenyl, 3,4,5-trimethoxyphenyl, 2,4-dimethoxyphenyl, 3,5-dimethoxyphenyl, 2-methyl-3-methoxyphenyl, 2-morpholinophenyl, 2-methoxy-4-methylphenyl, 2-dimethylaminophenyl, 4-dimethylaminophenyl, or

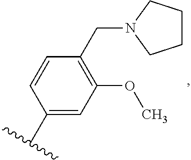

particularly phenyl; 2-methoxyphenyl; 3-methoxyphenyl; 2,3,4-trimethoxyphenyl; 3,4,5-trimethoxyphenyl; 2,4-dimethoxyphenyl; 3,5-dimethoxyphenyl; 2-methyl-3-methoxyphenyl; 2-morpholinophenyl; 2-methoxy-4-methylphenyl; 2-dimethylaminophenyl; or 4-dimethylaminophenyl.

In a further embodiment, the invention utilizes sirtuin-modulating compounds of Structural Formula (IX):

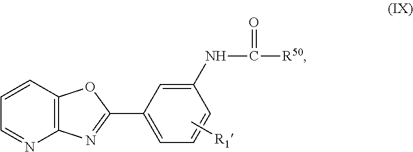

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention utilizes sirtuin-modulating compounds of Structural Formula (X):

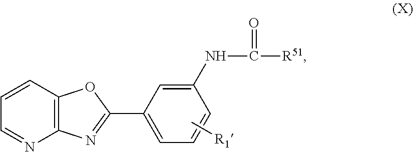

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo(b)thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In certain embodiments, R⁵¹ is selected from pyrazolyl, thiazolyl, oxazolyl, pyrimidinyl, furyl, thienyl, pyridyl, isoxazolyl, indolyl, benzopyrazolyl, benzothiazolyl, benzoxazolyl, quinoxalinyl, benzofuranyl, benzothienyl, quinolinyl, benzoisoxazolyl, benzotriazinyl, triazinyl, naphthyl, or

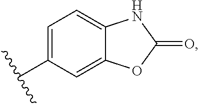

and wherein R⁵¹ is optionally substituted. In certain such embodiments, R⁵¹ is selected from pyrazolyl, thiazolyl, oxazolyl, pyrimidinyl, indolyl, pyrazinyl, triazinyl, or

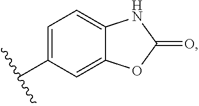

and R⁵¹ is optionally substituted.

In another aspect, the invention utilizes sirtuin-modulating compounds of Structural Formula (XI):

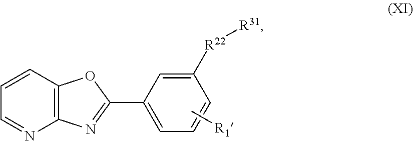

or a salt thereof, wherein:

R₁′ is selected from H or optionally substituted C₁-C₃ straight or branched alkyl;

R²² is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—-C(O)—NR₁′—, —CR₁′R₁′—C(O)—NR₁′—, —NR₁′—C(O)—CR₁′═CR₁′—CR₁′—, —NR₁′—C(═N—CN)—NR₁′—, —NR₁′—C(O)—CR₁′R₁′—O—, —NR₁′—C(O)—CR₁′R₁′—CR₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In certain embodiments, R²² is selected from —C(O)—NH—, —NH—, or —C(O)—NH—CH₃.

In certain embodiments, such as when R²² is selected from —C(O)—NH—, —NH—, or —C(O)—NH—CH₃, R³¹ is selected from optionally substituted phenyl, benzothiazolyl, quinoxalinyl, or benzoxazolyl.

In yet another aspect, the invention utilizes sirtuin-modulating compounds of Structural Formula (XII):

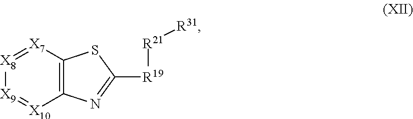

or a salt thereof, wherein:

each of X₇, X₈, X₉ and X₁₀ is independently selected from N, CR²⁰, or CR₁′, wherein:

each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

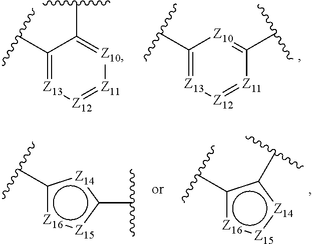

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,

with the proviso that when R¹⁹ is

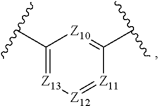

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:

each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

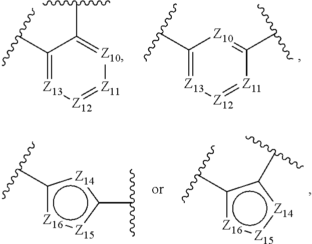

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —C(O)—NR₁′—S(O)₂—, —NR₁′—, —NR₁′—C(O)—CR₁′═CR₁′—, —NR₁′—C(O)—NR₁′—S(O)₂—, —NR₁′—C(O)—CR₁′═CR₁′—CR₁′R₁′—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

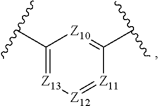

and each of Z₁₀, Z₁₁, Z₁₂ and Z₁₃ is independently selected from CR²⁰, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In certain embodiments, R²¹ is —NH—C(O)— and R¹⁹ is selected from:

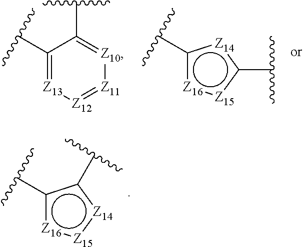

In certain embodiments, R¹⁹ is selected from optionally substituted phenyl, optionally substituted pyridyl, optionally substituted thienyl or optionally substituted furyl.

In certain embodiments, R¹⁹ is

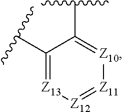

wherein each of Z₁₀, Z₁₁, Z₁₂ and Z₁₃ is independently selected from CR²⁰ or CR₁′; and

R²¹ is selected from —NH—C(O)—, —NH—C(O)—CH(CH₃)—O—, —NH—C(O)—CH₂—O—, or —NH—S(O)₂—CH₂—CH₂—; and

R³¹ is selected from an optionally substituted aryl, or an optionally substituted heteroaryl.

In certain such embodiments, R³¹ is optionally substituted with 1 to 3 substituents independently selected from —OCH₃, —CH₃, —N(CH₃)₂, phenyl, phenoxy, 3,4-dioxymethylene, fluoro, or another solubilizing group. Suitable examples of R³¹ include unsubstituted quinolinyl, 2,4-dimethoxyphenyl, 3,4-dimethoxyphenyl, 3,5-dimethoxyphenyl, 3,4,5-trimethoxyphenyl, 2,3,4-trimethoxyphenyl, 2-dimethylaminophenyl, 3-dimethylaminophenyl, 4-dimethylaminophenyl, 3,5-dimethylphenyl, 3,5-difluorophenyl, 3-trifluoromethoxyphenyl, unsubstituted quinoxalinyl, unsubstituted benzopyrimidinyl,

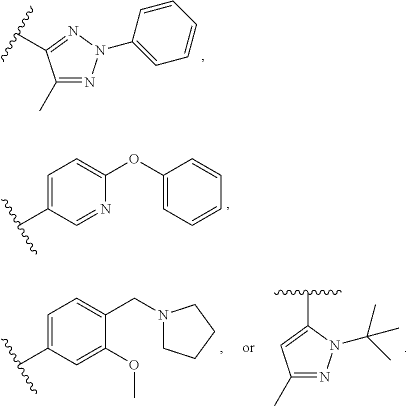

In certain such embodiments, R³¹ is not phenyl-substituted furyl.

In certain embodiments, R¹⁹ is selected from

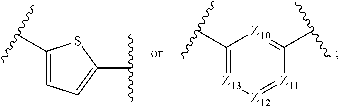

each of Z₁₀, Z₁₁, Z₁₂ and Z₁₃ is independently selected from CR²⁰, or CR₁′;

R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

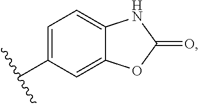

and R³¹ is optionally substituted (e.g., optionally substituted with up to three substituents independently selected from —OCH₃, —CH₃, —N(CH₃)₂, —O-phenyl, or another solubilizing group). Suitable examples of R³¹ include unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

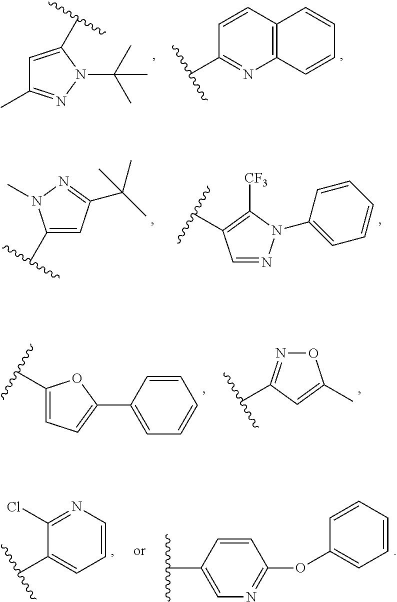

In certain embodiments, one or more of the following conditions applies:

when X₈ is N, R²¹ is —NH—C(S)—NH—, and R¹⁹ is phenyl, R³¹ is not 2-methoxy-5-nitrophenyl, 2-S-methylphenyl or 2-acetylphenyl;

when X₈ is N, R²¹ is —NH—S(O)₂—, and R¹⁹ is phenyl, R³¹ is not thiadiazole-substituted thienyl or 4-methylsulfonylphenyl;

when X₈ is N, R²¹ is —NH—CO—, and R¹⁹ is phenyl, R³¹ is not 2,4-difluorophenyl, pyridyl-substituted thienyl, 3,4-dichlorophenyl, 4-t-butylphenyl, or 3-benzyloxyphenyl;

when X₉ is N, R²¹ is —NH—C(O)— and R¹⁹ is

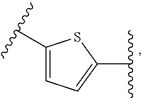

R³¹ is not 2,3,4-trimethoxyphenyl or 3,5-dimethoxyphenyl; and

when X₉ is N, R²¹ is —NH—C(O)— and R¹⁹ is phenyl, R³¹ is not 3,5-dimethoxyphenyl.

In a further embodiment, the invention utilizes compounds of Structural Formula (XIII):

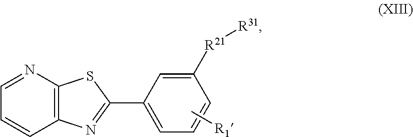

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethlyphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In certain embodiments, R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from a monocyclic or bicyclic aryl or a monocyclic or bicyclic heteroaryl, and comprises a solubilizing group substituent.

In certain embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

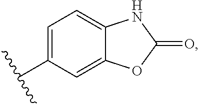

and R³¹ is optionally substituted.

In certain embodiments, R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, particularly when R²¹ is —NH—C(O)—, R³¹ is selected from R³¹ is selected from unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

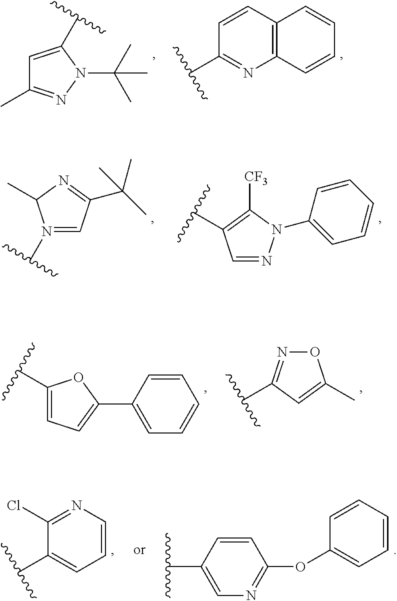

In one aspect, the invention provides sirtuin-modulating compounds of Structural Formula (XIV):

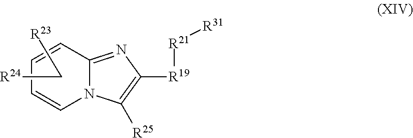

or a salt thereof, wherein:

each of R²³ and R²⁴ is independently selected from H, —CH₃ or a solubilizing group;

R²⁵ is selected from H or a solubilizing group; and

R¹⁹ is selected from:

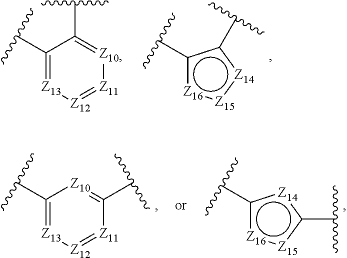

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, S, O, CR²⁰, or CR₁′, wherein:
   zero to two of Z₁₀, Z₁₁, Z₁₂ or Z₁₃ are N;
   at least one of Z₁₄, Z₁₅ and Z₁₆ is N, NR₁′, O or S;
   zero to one of Z₁₄, Z₁₅ and Z₁₆ is S or O;
   zero to two of Z₁₄, Z₁₅ and Z₁₆ are N or NV;
   zero to one R²⁰ is a solubilizing group; and
   zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R′₁—NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁′—, —NR₁′—S(O)₂—NR₁′, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R′₁—CR₁′R′₁—O—, —NR₁′—S(O)₂—CR₁′R′₁—, —NR₁′—S(O)₂—CR₁′R′₁—, —NR₁′—C(O)—CR₁′R′₁—CR₁′R′₁—, —NR₁′—C(S)—NR₁′—CR₁′R′₁—, —NR₁′—C(O)—O— or —NR₁′—C(O)—CR₁′R₁′—; and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,

wherein when R¹⁹ is

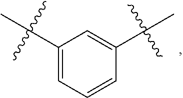

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In certain embodiments, each of R²³ and R²⁴ is independently selected from H, —CH₃ or a solubilizing group;

R²⁵ is selected from H, or a solubilizing group; and

R¹⁹ is selected from:

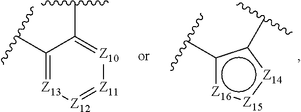

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,
wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R′₁—NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —CR₁′R′₁—C(O)—NR₁′—, —NR₁′—C(O)—CR₁′═CR₁′—CR₁′R₁′, —NR₁′—C(═N—CN)—NR₁′—, —NR₁′—C(O)—CR₁′R′₁—O—, —NR₁′—C(O)—CR₁′R′₁—CR₁′R′₁—O—, —NR₁′—S(O)₂—CR₁′R′₁—, —NR₁′—S(O)₂—CR₁′R′₁—CR₁′R′₁—, —NR₁′—C(O)—CR₁′R′₁—CR₁′R′₁—, —NR₁′—C(S)—NR₁′—CR₁′R′₁—CR₁′R′₁—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, R³¹ is not 2,4-dimethoxyphenyl.

Typically, R²⁵ is selected from H, —CH₂—N(CH₃)₂, or

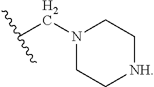

Typically, R²³ and R²⁴ are H.

Typically, R¹⁹ is selected from phenyl, pyridyl, thienyl or furyl, particularly optionally substituted phenyl. Preferably, a phenyl is optionally substituted with:

a) up to three —O—CH₃ groups; or

b) one —N(CH₃)₂ group.

In certain embodiments, each of R²³ and R²⁴ is independently selected from H, —CH₃ or a solubilizing group;

R²⁵ is selected from H, or a solubilizing group; and

R¹⁹ is selected from:

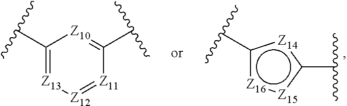

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
   zero to two of Z₁₀, Z₁₁, Z₁₂ or Z₁₃ are N;
   at least one of Z₁₄, Z₁₅ and Z₁₆ is N, NR₁′, O or S;
   zero to one of Z₁₄, Z₁₅ and Z₁₆ is S or O;
   zero to two of Z₁₄, Z₁₅ and Z₁₆ is N or NR₁′;
   zero to one R²⁰ is a solubilizing group; and
   zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁′—, —NR₁′—C(O)—CR₁′R′₁—NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R′₁—CR₁′R′₁—O—, —NR₁′—S(O)₂—CR₁′R′₁—, —NR₁′—S(O)₂—CR₁′R₁′, —CR₁′R′₁—, —NR₁′—C(O)—CR₁′R′₁—CR₁′R₁′, —, —NR₁′—C(S)—NR₁′—CR₁′R′₁—CR₁′R′₁—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,

wherein when R¹⁹ is phenyl, at least one of R²³, R²⁴, or R²⁵ is a solubilizing group and wherein said compound is not 2-chloro-N-[3-[3-(cyclohexylamino)imidazo[1,2-a]pyridin-2-yl]phenyl]-4-nitrobenzamide.

Typically, R²⁵ is selected from H, —CH₂—N(CH₃)₂, or

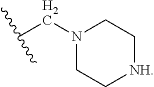

Typically, R²³ and R²⁴ are H.

Typically, R¹⁹ is selected from phenyl, pyridyl, thienyl or furyl, particularly optionally substituted phenyl. Preferably, a phenyl is optionally substituted with:

a) up to three —O—CH₃ groups; or

b) one —N(CH₃)₂ group.

In another aspect, the invention utilizes sirtuin-modulating compounds of Structural Formula (XV):

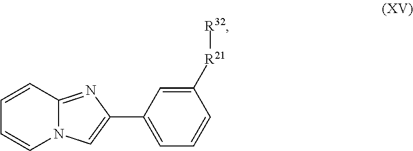

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R′₁—NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R′₁—CR₁′R′₁—O—, —NR₁′—S(O)₂—CR₁′R′₁—, —NR₁′—S(O)₂—CR₁′R′₁—CR₁′R′₁—, —NR₁′—C(O)—CR₁′R′₁—CR₁′R′₁—, —NR₁′—C(S)—NR₁′—CR₁′R′₁—CR₁′R′₁—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

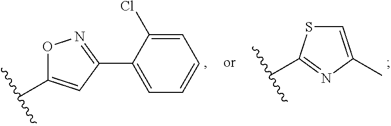

and

when R²¹ is —NR₁′—S(O)₂—, R³² is not unsubstituted 2-thienyl or unsubstituted naphthyl.

In yet another aspect, the invention provides sirtuin-modulating compounds of Structural Formula (XVI):

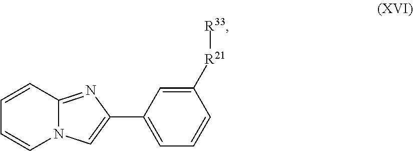

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R′₁—, —NR₁′—, —NR₁′—C(═NR₁′)—NR₁′—, —C(O)—NR₁′, —C(O)—NR₁′—S(O)₂—, —NR₁′—, —CR₁′R′₁—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —CR₁′R′₁—C(O)—NR₁′—, —NR₁′—C(O)—CR₁′R′₁—O—, —NR₁′—C(O)—CR₁′R′₁—, —CR₁′R′₁—O—, —NR₁′—S(O)₂—CR₁′R′₁—, —NR₁′—S(O)₂—CR₁′R′₁—CR₁′R′₁—, —NR₁′—C(O)—CR₁′═CR₁′—CR₁′R₁′—, —NR₁′—C(═N—CN)—NR₁′—, —NR₁′—C(O)—CR₁′R′₁—CR₁′R′₁—, —NR₁′—C(S)—NR₁′—CR₁′R′₁—CR₁′R′₁—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In certain embodiments, R²¹ is selected from —NR²²—C(O)—, —NR₁′—C(O)—NR₁′—, —NR₁′—C(S)—NR₁′—, —NR₁′—C(S)—NR₁′—CR₁′R′₁—, —NR₁′—C(O)—CR₁′R′₁—NR₁′—, —NR₁′—C(═NR₁′)—NR₁′—, —C(O)—NR₁′—, —C(O)—NR₁′—S(O)₂—, —CR₁′R′₁—, —NR₁′—S(O)₂—NR₁′—, —NR₁′—C(O)—NR₁′—S(O)₂—, —NR₁′—CR₁R′₁—C(O)—NR₁′—, —NR₁′—C(O)—CR₁′R′₁—O—, —NR₁′—S(O)₂—CR₁′R′₁—, —NR₁′—C(O)—CR₁′═CR₁′—CR₁′R₁′—, —NR₁′—C(═N—CN)—NR₁′—, or —NR₁′—C(O)—CR₁′R₁′—; and

each R₁′ is independently selected from H or optionally substituted C₁-C₃ straight or branched alkyl;

R²² is an optionally substituted C₁-C₃ straight or branched alkyl; and

R³³ is phenyl comprising a solubilizing group substituent, wherein: when R²¹ is —NH—S(O)₂ said phenyl comprises an additional substituent.

In certain embodiments, R²¹ is selected from —NR²²—C(O)—, —NR₁′—C(S)—NR₁′—, —NR₁′—C(O)—CR₁′R′₁—, —NR₁′—C(═NR₁′)—NR₁′—, —C(O)—NR₁′—, —C(O)—NR₁′—S(O)₂—, —CR₁′R′₁—, —NR₁′—C(O)—CR₁′═CR₁′—, —NR₁′—C(O)—NR₁′—S(O)₂—, —NR₁′—CR₁′R′₁—C(O)—NR₁′—, —NR₁′—S(O)₂—CR₁′R′₁—, —NR₁′—S(O)₂—CR₁′R′₁—, —NR₁′—C(O)—CR₁′═CR₁′—CR₁′R₁′—, or —NR₁′—C(═N—CN)—NR₁′—,

each R₁′ is independently selected from H or optionally substituted C₁-C₃ straight or branched alkyl; and

R²² is an optionally substituted C₁-C₃ straight or branched alkyl.

In certain embodiments, R³³ is optionally substituted on up to three carbon atoms with a substituent independently selected from —O—CH₃, —CH₃, —N(CH₃)₂, —S(CH₃), or CN; or substituted on adjacent carbon atoms with

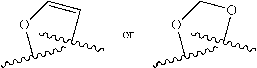

bridging said adjacent carbon atoms.

In a further embodiment, the invention utilizes sirtuin-modulating compounds of Structural Formula (XVII):

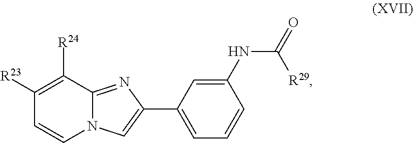

or a salt thereof, wherein:

each of R²³ and R²⁴ is independently selected from H or —CH₃, wherein at least one of R²³ and R²⁴ is H; and

R²⁹ is phenyl substituted with:
a) two —O—CH₃ groups;
b) three —O—CH₃ groups located at the 2,3 and 4 positions; or
c) one —N(CH₃)₂ group; and;
d) when R²³ is CH₃, one —O—CH₃ group at the 2 or 3 position,

wherein R²⁹ is optionally additionally substituted with a solubilizing group.

In certain embodiments, R²⁹ is phenyl substituted with:
a) three —O—CH₃ groups located at the 2,3 and 4 positions; or
b) one —N(CH₃)₂ group.

In one aspect, the invention utilizes sirtuin-modulating compounds of Structural Formula (XVIII):

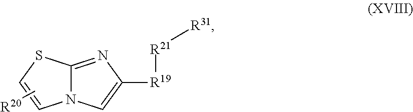

or a salt thereof, wherein

R¹⁹ is selected from:

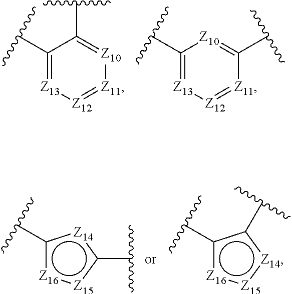

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,
wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′, —C(O)—NR₁′—S(O)₂—, —NR₁′—, —CR₁′R₁′—, —NR₁′—C(O)—CR₁′═CR₁′, —NR₁′—S(O)₂—NR₁′—, —NR₁′—C(O)—CR₁′═CR₁′—CR₁′—, —NR₁′—C(═N—CN)—NR₁′—, —NR₁′—C(O)—CR₁′R₁′—O—, —NR₁′—C(O)—CR₁′═CR₁′—O—, —NR₁′—S(O)₂—CR₁′—, —NR₁′—S(O)₂—CR₁′R₁′—, —NR₁′—C(O)—CR₁′—; —NR₁′—C(S)—NR₁′—CR₁′R′₁—CR₁′R′₁—, —NR₁′—C(O)—O—,

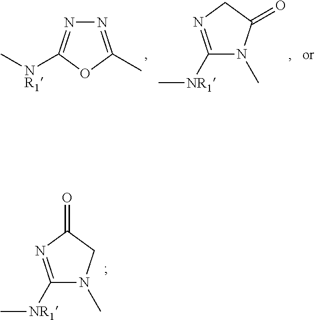

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, with the proviso that when R¹⁹ is

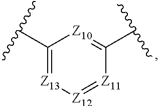

Z₁₀, Z₁₁, Z₁₂ and Z₁₃ are each CH, R²⁰ is H, and R²¹ is —NHC(O)—, R³¹ is not an optionally substituted phenyl.
In certain embodiments, R¹⁹ is selected from:

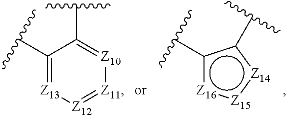

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,
wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

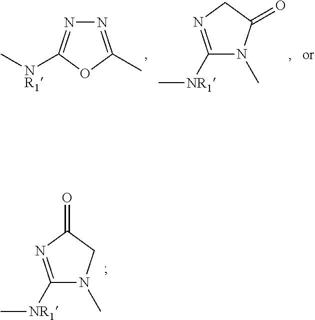

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, compounds of Structural Formula (XVIII) have the formula:

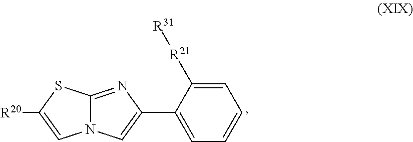

or a salt thereof, wherein

R²⁰ is selected from H or a solubilizing group;

R²¹ is selected from —NH—C(O)—, or —NH—C(O)—CH₂—; and

R³¹ is selected from an optionally substituted monocyclic or bicyclic aryl, or an optionally substituted monocyclic or bicyclic heteroaryl.

Typically, R¹⁹ in compounds of Structural Formula (XVIII) is selected from phenyl, pyridyl, thienyl or furyl, particularly optionally substituted phenyl.

Typically, R²⁰ is selected from H, —CH₂—N(CH₃)₂,

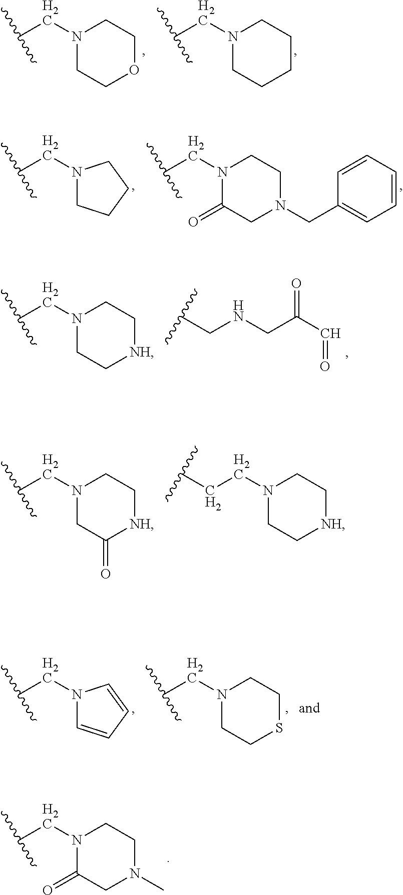

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CF₁₂—.

In certain such embodiments, when R²¹ is —NR₁′—C(O)—, R³¹ is not 4-cyanophenyl or

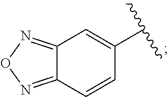

and/or when R²¹ is —NR₁′—S(O)₂—, R³¹ is not 4-methoxyphenyl or 4-t-butylphenyl.

In certain such embodiments, when R¹⁹ is

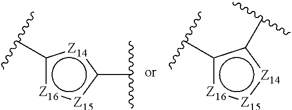

and R²¹ is —NR₁′—C(O)—, R³¹ is not 4-cyanophenyl or

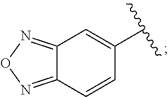

and/or when R¹⁹ is

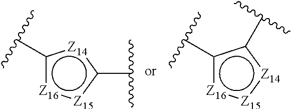

and R²¹ is —NR₁′—S(O)₂—, R³¹ is not 4-methoxyphenyl or 4-t-butylphenyl.

In another aspect, the invention utilizes sirtuin-modulating compounds of Structural Formula (XX):

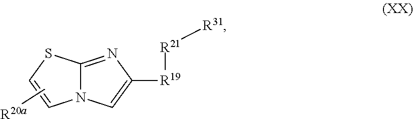

or a salt thereof, wherein

R¹⁹ is selected from:

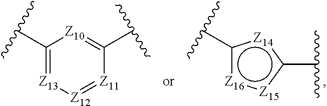

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,
wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R^{20a} is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R₁′—CR₁′R′₁—, —NR₁′—C(O)—O—,

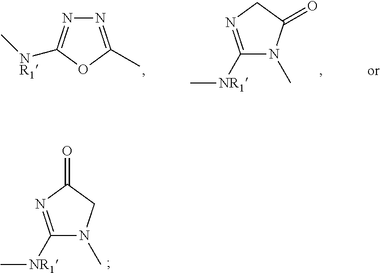

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

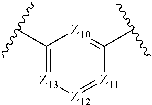

and Z₁₀, Z₁₁, Z₁₂ and Z₁₃ are each CH, R^{20a} is a solubilizing group.

Typically, R¹⁹ in compounds of Structural Formula (XX) is selected from phenyl, pyridyl, thienyl or furyl, particularly optionally substituted phenyl.

Typically, R^{20a} is selected from H, —CH₂—N(CH₃)₂,

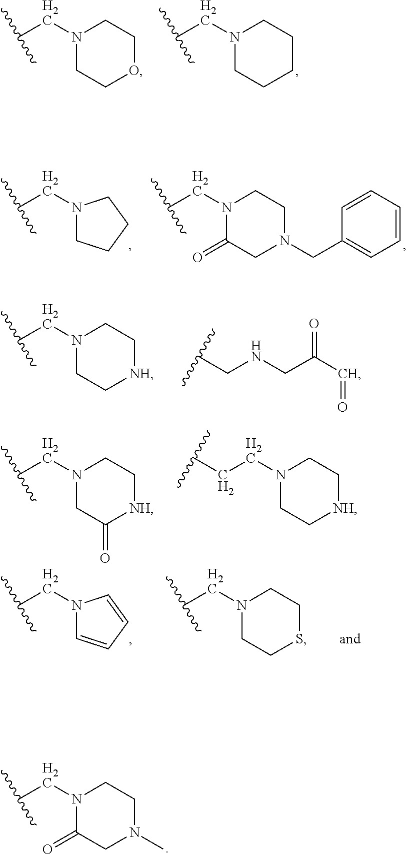

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In yet another aspect, the invention utilizes sirtuin-modulating compounds of Structural Formula (XXI):

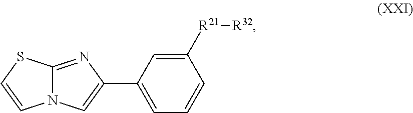

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

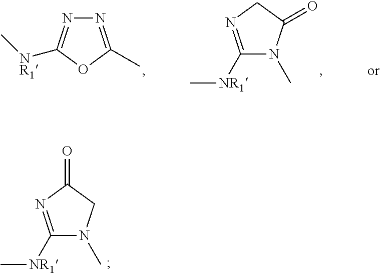

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In certain embodiments, R³² is selected from pyrrolyl, pyrazolyl, pyrazinyl, furyl, pyridyl, pyrimidinyl, or thienyl, and R³² is optionally substituted and is optionally benzofused.

In certain embodiments, R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

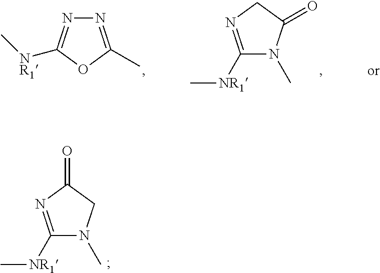

wherein
each R₁′ is independently selected from H or optionally substituted C₁-C₃ straight or branched alkyl; and

R³² is selected from benzofuryl, methylfuryl, benzothienyl, pyridyl, pyrazinyl, pyrimidinyl, pyrazolyl, wherein said methyfuryl, pyridyl, pyrazinyl, pyrimidinyl or pyrazolyl is optionally benzofused and wherein R³² is optionally substituted or further substituted.

In a further aspect, the invention provides sirtuin-modulating compounds of Structural Formula (XXII):

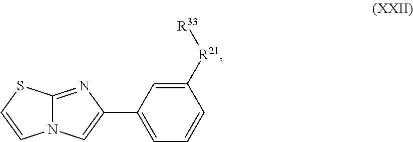

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—C(O)—CR₁′R₁′—O—, —NR₁′—S(O)₂—CR₁′R₁′—, —NR₁′—S(O)₂—CR₁′R₁′—CR₁′R₁′—,

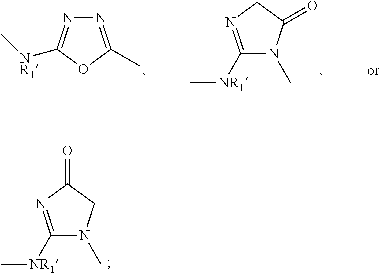

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

Preferably, R²¹ is selected from —NH—C(O)— or —NH—C(O)—CH₂—.

In one aspect, the invention utilizes sirtuin-modulating compounds of Structural Formula (XXII):

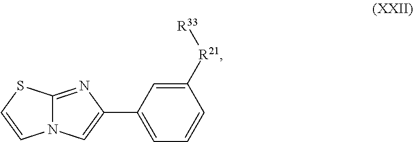

or a salt thereof wherein:
R²¹ is selected from —NH—C(O)—, or —NH—C(O)—CH₂—; and
R³³ is phenyl substituted with

a) one —N(CH₃)₂ group;

b) one CN group at the 3 position;

c) one —S(CH₃) group; or

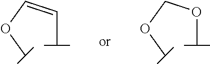

bridging the 3 and 4 positions.

In another aspect, the invention utilizes sirtuin-modulating compounds of Structural Formula (XXIII):

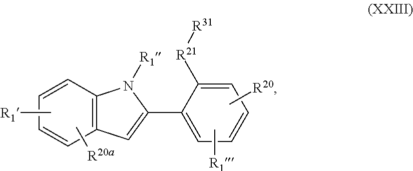

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′—-CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl,

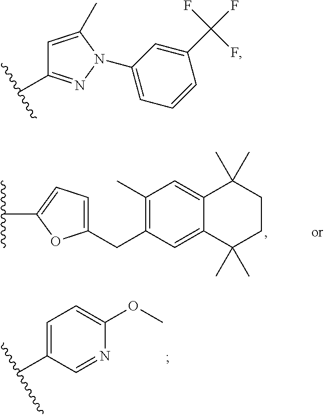

when R²¹ is —NH—C(O)— and each of R²⁰, R^{20a}, R₁′, R₁″ and R₁′″ is hydrogen, R³¹ is not

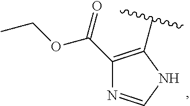

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;

when R²¹ is —NH—C(O)—, R₁″ is methyl; and each of R²⁰, R^{20a}, R₁′ and R₁′″ is hydrogen, R³¹ is not 2-methylaminophenyl,

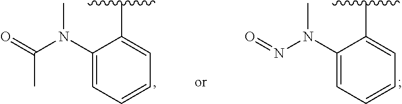

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;

when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

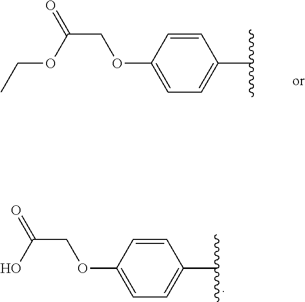

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In a particular aspect, the invention utilizes sirtuin-modulating compounds of Structural Formula (XXIII):

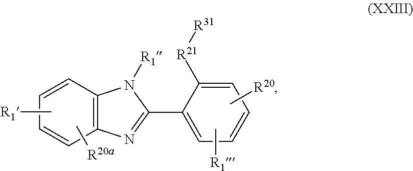

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,

wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In yet another aspect, the invention provides sirtuin-modulating compounds of Structural Formula (XXIV):

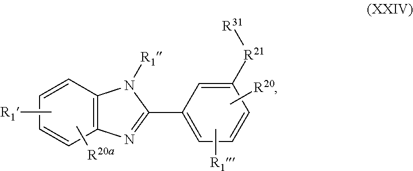

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl;
R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—CH₂—, R³¹ is not 2-methylphenyl, or 3,4-dimethoxyphenyl;
when R²¹ is —NH—C(O)—CH═CH—, R³¹ is not 2-chlorophenyl;
when R²¹ is —NH—C(O)—NH—, R³¹ is not unsubstituted benzimidazolyl;
when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;
when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;
when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;
when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

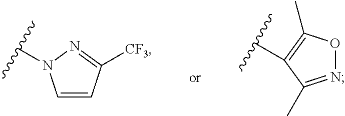

when R²¹ is —NH—C(O)—CH₂—, R₁′ is methyl, and each of R²⁰, R^{20a}, R₁″, and R₁′″ is hydrogen, R³¹ is not unsubstituted phenyl;
when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;
when R²¹ is —NH—C(O)—CH(CH₂CH₃)—, and each of R²⁰, R^{20a}, R₁′, R₁″, and R₁′″ is hydrogen, R³¹ is not unsubstituted phenyl;
when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention utilizes sirtuin-modulating compounds of Structural Formula (XXIV):

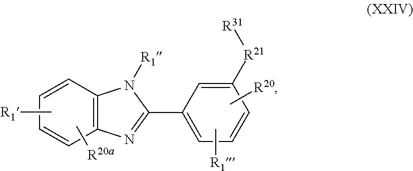

or a salt thereof, wherein:
each R²⁰ and R^{20a} is independently selected from H or a solubilizing group and at least one of R²⁰ and R^{20a} is a solubilizing group;
each R₁′, R₁″ and R₁′″ is independently selected from H or optionally substituted C₁-C₃ straight or branched alkyl;
R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁1-CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and
R³¹ is selected from an optionally substituted monocyclic or bicyclic aryl, or an optionally substituted monocyclic or bicyclic heteroaryl.

In certain embodiments, when R²¹ is —NH—C(O)—CH₂—, R³¹ is not 2-methylphenyl; or 3,4-dimethoxyphenyl; when R²¹ is —NH—C(O)—CH═CH—, R³¹ is not 2-chlorophenyl; and/or when R²¹ is —NH—C(O)—NH—, R³¹ is not unsubstituted benzimidazolyl.

In a further aspect, the invention utilizes sirtuin-modulating compounds of Structural Formula (XXV):

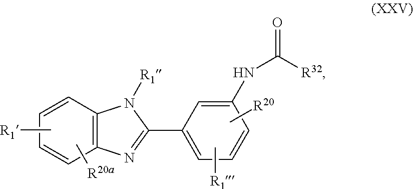

or a salt thereof, wherein:
each R²⁰ and R^{20a} is independently selected from H or a solubilizing group, wherein at least one of R²⁰ and R^{20a} is a solubilizing group;
each R₁′, R₁″ and R₁′″ is independently selected from H or optionally substituted C₁-C₃ straight or branched alkyl; and
R³² is an optionally substituted phenyl.

In certain embodiments, R³² is selected from 3,4-dimethoxyphenyl, 2,6-dimethoxyphenyl, or 2,4-dimethoxyphenyl; wherein R³² is further optionally substituted with a solubilizing group.

In certain embodiments, R³² is not unsubstituted thienyl; unsubstituted phenyl; 2-methylphenyl; 4-fluorophenyl; 4-methoxyphenyl; 4-methylphenyl; 3,4-dioxyethylenephenyl; 3-acetylamino-4-methylphenyl; 3-[(6-amino-1-oxohexyl)amino]-4-methylphenyl; 3-amino-4-methylphenyl; 3,5-dimethoxyphenyl; 3-halo-4-methoxyphenyl; 3-nitro-4-methylphenyl; or 4-propoxyphenyl.

In one aspect, the invention provides sirtuin-modulating compounds of Structural Formula (XXVI):

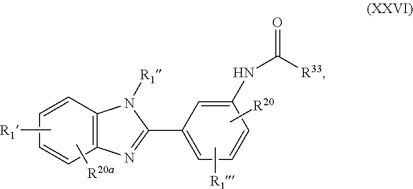

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl; and
R³³ is selected from an optionally substituted heteroaryl or an optionally substituted bicyclic aryl, with the provisos that:
when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

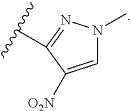

In a particular aspect, the invention utilizes sirtuin-modulating compounds of Structural Formula (XXVI):

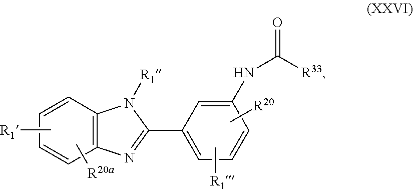

or a salt thereof, wherein:
each R²⁰ and R^{20a} is independently selected from H or a solubilizing group, wherein at least one of R²⁰ or R^{20a} is a solubilizing group;
each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention utilizes sirtuin-modulating compounds of Structural Formula (XXVII):

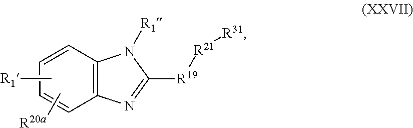

wherein:

each R²⁰ and R^{20a} is independently selected from H or a solubilizing group;
each R₁′ and R₁″ is independently selected from H or optionally substituted C₁-C₃ straight or branched alkyl;

R¹⁹ is selected from:

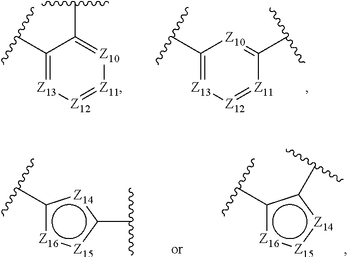

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—-C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R′₁—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

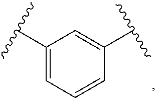

R³¹ is not unsubstituted pyridyl, 2,6-dimethoxyphenyl, 3,4,5-trimethoxyphenyl or unsubstituted furyl.

In a particular aspect, the invention utilizes sirtuin-modulating compounds of Structural Formula (XXVII):

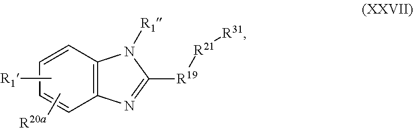

or a salt thereof, wherein:
each R²⁰ and R^{20a} is independently selected from H or a solubilizing group;
each R₁′ and R₁″ is independently selected from H or optionally substituted C₁-C₃ straight or branched alkyl;
R¹⁹ is selected from:

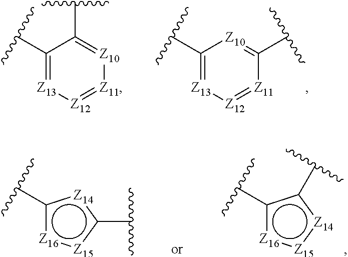

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—, and R¹⁹ is thiazolyl, R³¹ is not optionally substituted phenyl or optionally substituted pyridyl;

when R²¹ is —NH—C(O)—CH₂—, and R¹⁹ is pyrazolyl, R³¹ is not unsubstituted indolyl or unsubstituted phenyl;

when R²¹ is —NH—C(O)—CH₂—, and R¹⁹ is

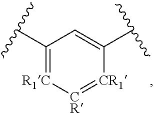

R³¹ is not 2-methylphenyl or 3,4-dimethoxyphenyl;

when R²¹ is —NH—C(O)—CH═CH—, and R¹⁹ is

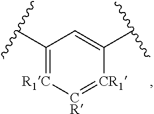

R³¹ is not 2-chlorophenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;

when R²¹ is —NH—C(O)—NH—, and R¹⁹ is

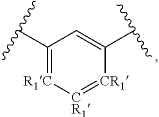

R³¹ is not unsubstituted benzimidazolyl;

when R²¹ is —NH—, and R¹⁹ is pyrazolyl, R³¹ is not unsubstituted pyridyl;

when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;

when R^{20a} is a solubilizing group, R¹⁹ is thienyl and R²′ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;

when R²¹ is —NH— and R¹⁹ is pyridyl, oxadiazolyl or thiadiazolyl, R³′ is not unsubstituted phenyl, 3-methoxyphenyl or 4-methoxyphenyl;

when R²¹ is —NH—C(O)— and R¹⁹ is thiazolyl or pyrimidinyl, R³¹ is not unsubstituted phenyl;

when R²¹ is —NH—C(O)— and R¹⁹ is

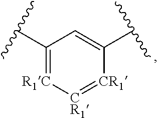

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

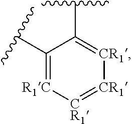

R³¹ is not 3,5-dinitrophenyl, 4-butoxyphenyl,

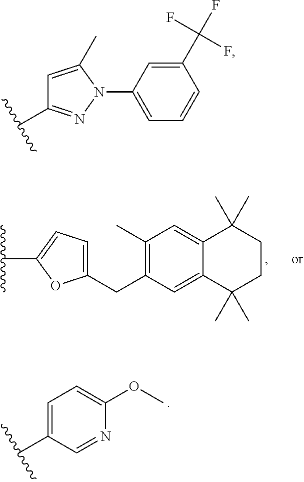

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In certain embodiments, when R²¹ is —NH—C(O)—, R¹⁹ is not

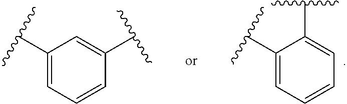

In certain embodiments, when R²¹ is —NH—C(O)—, R¹⁹ is not optionally substituted pyrazolyl, thiazolyl, thienyl, pyrrolyl or pyrimidinyl; when R²¹ is —NH—C(O)—CH₂— or —NH—C(O)—NH—, R¹⁹ is not pyrazolyl; and/or when R²¹ is —NH—, R¹⁹ is not optionally substituted pyridyl, thiazolyl, pyrazolyl, thiadiazolyl, or oxadiazolyl.

In a more particular aspect, the invention utilizes sirtuin-modulating compounds of Structural Formula (XXVII):

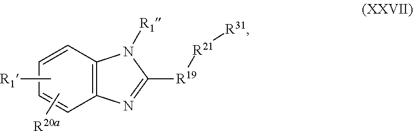

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R¹⁹ is selected from:

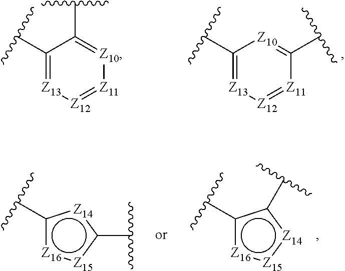

wherein:
each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

one to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—C(O)—CH₂—, and R¹⁹ is pyrazolyl, R³¹ is not unsubstituted indolyl or unsubstituted phenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;

when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and

when R²¹ is —NH—C(O)— and R¹⁹ is thiazolyl or pyrimidinyl, R³¹ is not unsubstituted phenyl.

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In yet another aspect, the invention utilizes compounds of Structural Formula (XXVIII):

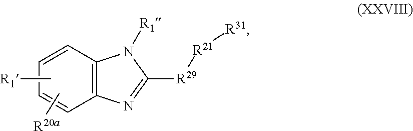

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R²⁹ is selected from:

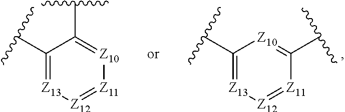

wherein:

each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl.

In certain embodiments, R³¹ is optionally substituted phenyl, such as 3-methoxyphenyl, 3,4-dimethoxyphenyl, 3,4,5-trimethoxyphenyl, or 4-dimethylaminophenyl.

In certain embodiments, R²¹ is —NH—C(O)—.

In preferred embodiments, R²¹ is —NH—C(O)— and R³¹ is an optionally substituted phenyl, such as 3-methoxyphenyl, 3,4-dimethoxyphenyl, 3,4,5-trimethoxyphenyl, or 4-dimethylaminophenyl.

In a further aspect, such as when the sirtuin modulator is a sirtuin inhibitor, the invention provides novel sirtuin-modulating compounds of Formula (VI):

or a salt thereof, wherein:

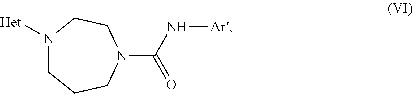

Het is an optionally substituted heterocyclic aryl group; and

Ar′ is an optionally substituted carbocyclic or heterocyclic aryl group.

In certain embodiments, Het comprises one N heteroatom and 1 to 2 additional heteroatoms independently selected from N, O or S, such as oxazolopyridyl.

In certain embodiments, Ar′ is selected from optionally substituted phenyl, benzothiazolyl, or benzoxazolyl. When Ar′ is substituted phenyl, typically it is substituted with 1 to 3 substituents independently selected from halo, methyl, O-methyl, S-methyl or N(CH₃)₂, morpholino, or 3,4 dioxymethylene.

Compounds described above can be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

In the compounds described above, bivalent groups disclosed as possible values for variables can have either orientation, provided that such orientation results in a stable molecule. Preferably, however, the left hand side of a bivalent group (e.g., —NR₁′—C(O)—) is attached to a bivalent arylene or heteroarylene group (e.g., R¹⁹) and the right hand side of a bivalent group is attached to a monovalent aryl group (e.g., R³¹).

Sirtuin-modulating compounds of the invention having hydroxyl substituents, unless otherwise indicated, also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OSO₃⁻M⁺, where M⁺ is a suitable cation (preferably H⁺, NH₄⁺ or an alkali metal ion such as Na⁺ or K⁺) and sugars such as

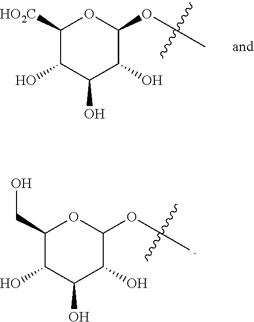

These groups are generally cleavable to —OH by hydrolysis or by metabolic (e.g., enzymatic) cleavage.

In certain embodiments, the compounds of the invention exclude one or more of the species disclosed in Tables 4-6. In certain such embodiments, the compounds of the invention exclude compound 7.

Sirtuin-modulating compounds of the invention advantageously modulate the level and/or activity of a sirtuin protein, particularly the deacetylase activity of the sirtuin protein.

Separately or in addition to the above properties, certain sirtuin-modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity, at concentrations of the compound that are effective for modulating the deacetylation activity of a sirtuin protein (e.g., such as a SIRT1 and/or a SIRT3 protein).

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A ring (e.g., 5- to 7-membered ring) or cyclic group includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuryl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR₁′CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}—R^{d} are each independently an aliphatic, substituted aliphatic, benzyl, substituted benzyl, aromatic or substituted aromatic group, preferably an alkyl, benzylic or aryl group. In addition, —NR^{a}R^{b}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

A hydrogen-bond donating group is a functional group having a partially positively-charged hydrogen atom (e.g., —OH, —NH₂, —SH) or a group (e.g., an ester) that metabolizes into a group capable of donating a hydrogen bond.

As used herein, a “solubilizing group” is a moiety that has hydrophilic character sufficient to improve or increase the water-solubility of the compound in which it is included, as compared to an analog compound that does not include the group. The hydrophilic character can be achieved by any means, such as by the inclusion of functional groups that ionize under the conditions of use to form charged moieties (e.g., carboxylic acids, sulfonic acids, phosphoric acids, amines, etc.); groups that include permanent charges (e.g., quaternary ammonium groups); and/or heteroatoms or heteroatomic groups (e.g., O, S, N, NH, N—(CH₂)_{y}—R^{a}, N—(CH₂)_{y}—C(O)R^{a}, N—(CH₂)_{y}—C(O)OR^{a}, N—(CH₂)_{y}—S(O)₂R^{a}—, N—(CH₂)_{y}—S(O)₂OR^{a}, N—(CH₂)_{y}—C(O)NR^{a}R^{a}, etc., wherein R^{a} is selected from hydrogen, lower alkyl, lower cycloalkyl, (C6-C14) aryl, phenyl, naphthyl, (C7-C20) arylalkyl and benzyl, wherein R^{a} is optionally substituted; and y is an integer ranging from 0 to 6), optionally substituted heterocyclic groups (e.g., —(CH₂)ₙ—R^{b}, —(CH₂)ₙ—C(O)—R^{b}, —(CH₂)ₙ—O—(CH₂)ₙ—R^{b}, wherein R^{b} is selected from an optionally substituted saturated monocyclic heterocycle, an optionally substituted saturated bicyclic fused heterocycle, an optionally substituted saturated bicyclic spiro heterocycle, an optionally substituted heteroaryl and an optionally substituted partially substituted non-aryl heterocycle; and n is an integer ranging from 0 to 2). It should be understood that substituents present on R^{a} or R^{b} need not improve or increase water solubility over their unsubstituted counterparts to be within the scope of this definition. All that is required is that such substituents do not significantly reverse the improvement in water-solubility afforded by the unsubstituted R^{a} or le moiety.

In one embodiment, the solubilizing group increases the water-solubility of the corresponding compound lacking the solubilizing group at least 5-fold, preferably at least 10-fold, more preferably at least 20-fold and most preferably at least 50-fold.

In one preferred embodiment, the solubilizing group is a moiety of the formula:

—(CH₂)ₙ—R¹⁰⁰—N(R¹⁰¹)(R¹⁰¹), wherein:

n is selected from 0, 1 or 2;

R¹⁰⁰ is selected from a bond, —C(O)—, or —O(CH₂)ₙ; and

each R¹⁰¹ is independently selected from:
a. hydrogen;
b. C₁-C₄ straight or branched alkyl, wherein said alkyl is optionally substituted with halo, CN, OH, O—(C₁-C₄ straight or branched alkyl), N(R₁′)(R₁′), or ═O;

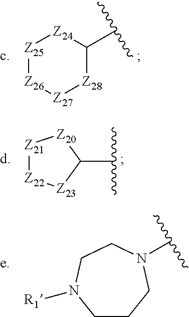

or
f. both R¹⁰¹ moieties are taken together with the nitrogen atom to which they are bound to form a ring of the structure

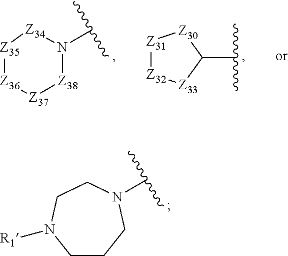

or
g. both R¹⁰¹ moieties are taken together with the nitrogen atom to which they are bound to form a 5-membered heteroaryl ring containing 1 to 3 additional N atoms, wherein said heteroaryl ring is optionally substituted with R₁′;
   wherein:
      each Z is independently selected from —O—, —S—, —NR₁′—, or —C(R⁵⁰)(R⁵⁰)—, wherein:
         at least three of Z₂₀, Z₂₁, Z₂₂, and Z₂₃ are —C(R⁵⁰ (R⁵⁰)—;
         at least three of Z₂₄, Z₂₅, Z₂₆, Z₂₇, and Z₂₈ are —C(R⁵⁰)(R⁵⁰)—;
         at least four of Z₃₀, Z₃₁, Z₃₂, and Z₃₃ are —C(R⁵⁰)(R⁵⁰)—; and
         at least four of Z₃₄, Z₃₅, Z₃₆, Z₃₇, and Z₃₈ are —C(R⁵⁰)(R⁵⁰)—;
      each R₁′ is independently selected from hydrogen or a C₁-C₃ straight or branched alkyl optionally substituted with one or more substituent independently selected from halo, —CN, —OH, —OCH₃, —NH₂, —NH(CH₃), —N(CH₃)₂, or ═O;
      each R⁵⁰ is independently selected from R₁′, halo, CN, OH, O—(C₁-C₄ straight or branched alkyl), N(R₁′)(R₁′), ═CRC, SR₁′, ═NR₁′, ═NOR₁′, or ═O;
      any two suitable non-cyclic R⁵⁰ are optionally bound to one another directly or via a C₁ to C₂ alkylene, alkenylene or alkanediylidene bridge to produce a bicyclic fused or spiro ring; and

any

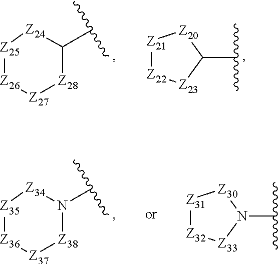

ring structure is optionally benzofused or fused to a monocyclic heteroaryl to produce a bicyclic ring.

For clarity, the term “C₁ to C₂ alkylene, alkenylene or alkanediylidene bridge” means the multivalent structures —CH₂—, —CH₂—CH₂—, —CH═, ═CH—, —CH═CH—, or ═CH—CH═. The two R⁵⁰ moieties that are optionally bound to one another can be either on the same carbon atom or different carbon atoms. The former produces a spiro bicyclic ring, while the latter produces a fused bicyclic ring. It will be obvious to those of skill in the art that when two R⁵⁰ are bound to one another to form a ring (whether directly or through one of the recited bridges), one or more terminal hydrogen atoms on each R⁵⁰ will be lost. Accordingly, a “suitable non-cyclic R⁵⁰” moiety available for forming a ring is a non-cyclic R⁵⁰ that comprises at least one terminal hydrogen atom.

In another preferred embodiment, the solubilizing group is a moiety of the formula: —(CH₂)ₙ—O—R¹⁰¹, wherein n and R¹⁰¹ are as defined above.

In another preferred embodiment, the solubilizing group is a moiety of the formula: —(CH₂)ₙ—C(O)—R₁′, wherein n and R₁′ are as defined above.

In a more preferred embodiment, a solubilizing group is selected from —(CH₂)ₙ—R¹⁰², wherein n is 0, 1 or 2; and R¹⁰² is selected from

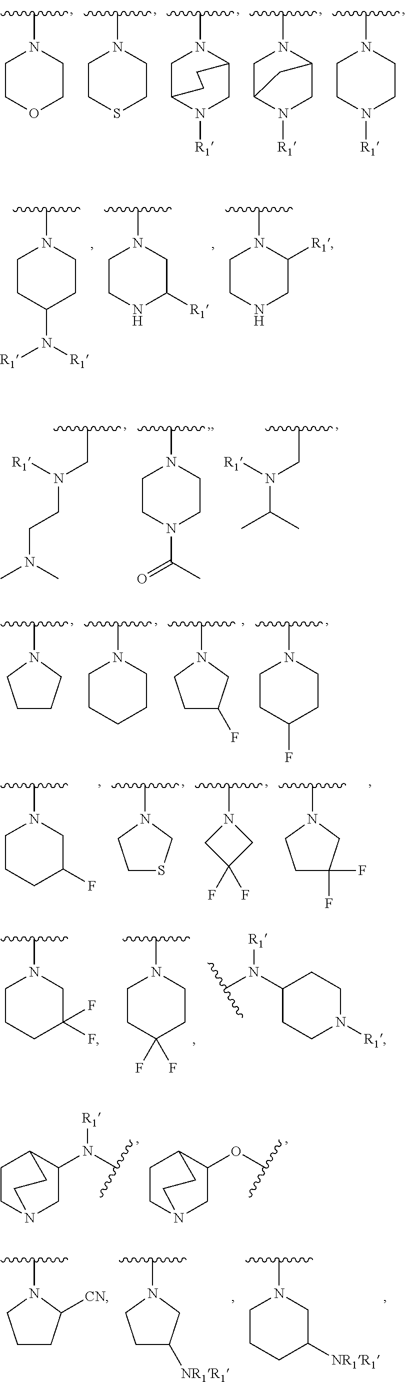

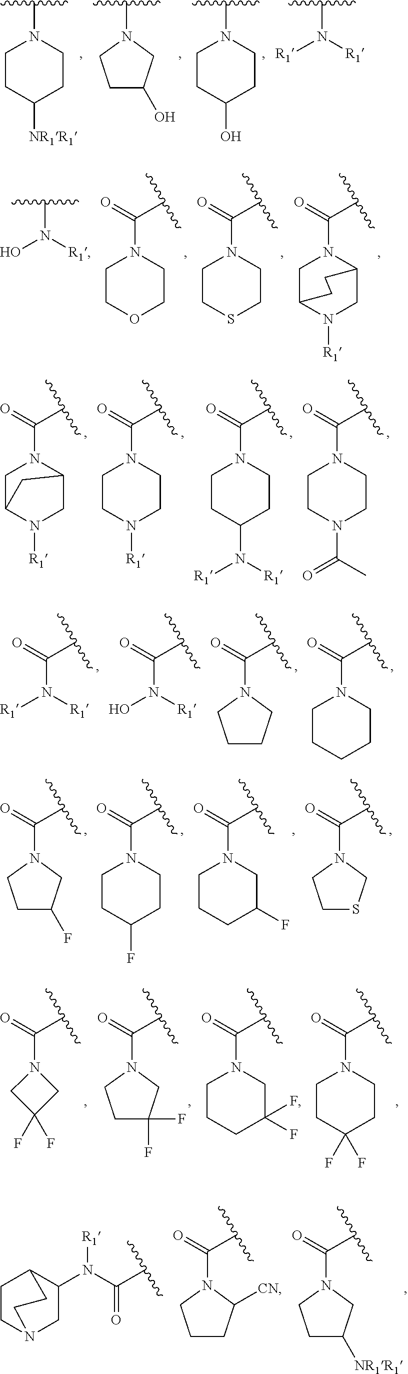

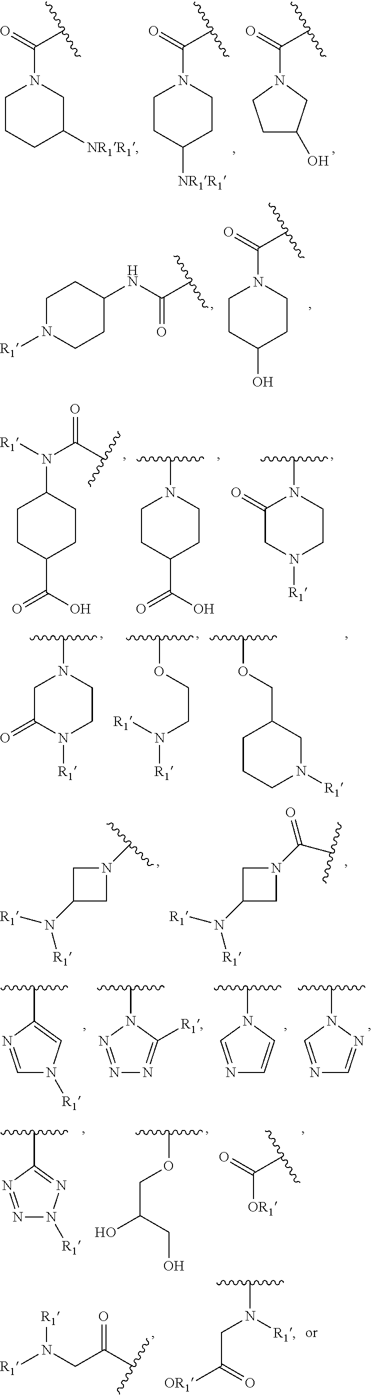

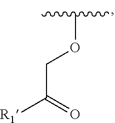

wherein R₁′ are as defined above.

In an even more preferred embodiment, a solubilizing group is selected from 2-dimethylaminoethylcarbamoyl, piperazin-1-ylcarbonyl, piperazinylmethyl, dimethylaminomethyl, 4-methylpiperazin-1-ylmethyl, 4-aminopiperidin-1-yl-methyl, 4-fluoropiperidin-1-yl-methyl, morpholinomethyl, pyrrolidin-1-ylmethyl, 2-oxo-4-benzylpiperazin-1-ylmethyl, 4-benzylpiperazin-1-ylmethyl, 3-oxopiperazin-1-ylmethyl, piperidin-1-ylmethyl, piperazin-1-ylethyl, 2,3-dioxopropylaminomethyl, thiazolidin-3-ylmethyl, 4-acetylpiperazin-1-ylmethyl, 4-acetylpiperazin-1-yl, morpholino, 3,3-difluoroazetidin-1-ylmethyl, 2H-tetrazol-5-ylmethyl, thiomorpholin-4-ylmethyl, 1-oxothiomorpholin-4-ylmethyl, 1,1-dioxothiomorpholin-4-ylmethyl, 1H-imidazol-1-ylmethyl, 3,5-dimethylpiperazin-1-ylmethyl, 4-hydroxypiperidin-1-ylmethyl, N-methyl(1-acetylpiperidin-4-yl)-aminomethyl, N-methylquinuclidin-3-ylaminomethyl, 1H-1,2,4-triazol-1-ylmethyl, 1-methylpiperidin-3-yl-oxymethyl, or 4-fluoropiperidin-1-yl.

To the extent not included within any of the definitions set forth above, the term “solubilizing group” also includes moieties disclosed as being attached to the 7-position of 1-cyclopropyl-6-fluoro-1,4-dihydro-4-oxoquinoline-3-carboxylic acid (ciprofloxacin) and its derivatives, as disclosed in PCT publications WO 2005026165, WO 2005049602, and WO 2005033108, and European Patent publications EP 0343524, EP 0688772, EP 0153163, EP 0159174; as well as “water-solubilizing groups” described in United States patent publication 2006/0035891. The disclosure of each of these patent publications is incorporated herein by reference.

Double bonds indicated in a structure as:

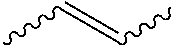

are intended to include both the (E)- and (Z)-configuration. Preferably, double bonds are in the (E)-configuration.

A sugar is an aldehyde or ketone derivative of a straight-chain polyhydroxy alcohol, which contains at least three carbon atoms. A sugar can exist as a linear molecule or, preferably, as a cyclic molecule (e.g., in the pyranose or furanose form). Preferably, a sugar is a monosaccharide such as glucose or glucuronic acid. In embodiments of the invention where, for example, prolonged residence of a compound derivatized with a sugar is desired, the sugar is preferably a non-naturally occurring sugar. For example, one or more hydroxyl groups are substituted with another group, such as a halogen (e.g., chlorine). The stereochemical configuration at one or more carbon atoms can also be altered, as compared to a naturally occurring sugar. One example of a suitable non-naturally occurring sugar is sucralose.

A fatty acid is a carboxylic acid having a long-chained hydrocarbon moiety. Typically, a fatty acid has an even number of carbon atoms ranging from 12 to 24, often from 14 to 20. Fatty acids can be saturated or unsaturated and substituted or unsubstituted, but are typically unsubstituted. Fatty acids can be naturally or non-naturally occurring. In embodiments of the invention where, for example, prolonged residence time of a compound having a fatty acid moiety is desired, the fatty acid is preferably non-naturally occurring. The acyl group of a fatty acid consists of the hydrocarbon moiety and the carbonyl moiety of the carboxylic acid functionality, but excludes the —OH moiety associated with the carboxylic acid functionality.

Also included in the present invention are salts, particularly pharmaceutically acceptable salts, of the sirtuin-modulating compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion (e.g., a halide such as bromide, chloride, or fluoride, particularly bromide).

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

Methods of producing the above-defined sirtuin-modulating compounds are described in U.S. Pat. No. 7,345,178. The compounds may be synthesized using conventional techniques. Advantageously, these compounds are conveniently synthesized from readily available starting materials.

Synthetic chemistry transformations and methodologies useful in synthesizing the sirtuin-modulating compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

In an exemplary embodiment, a sirtuin-modulating compound may traverse the cytoplasmic membrane of a cell and/or the blood-brain barrier. For example, a compound may have a cell-permeability or blood-brain barrier permeability of at least about 1%, 2%, 5%, 10%, 20%, 50%, 75%, 80%, 90% or 95%.

Sirtuin-modulating compounds described herein may also have one or more of the following characteristics: the compound may be essentially non-toxic to a cell or subject; the sirtuin-modulating compound may be an organic molecule or a small molecule of 2000 amu or less, 1000 amu or less; a compound may have a half-life under normal atmospheric conditions of at least about 30 days, 60 days, 120 days, 6 months or 1 year; the compound may have a half-life in solution of at least about 30 days, 60 days, 120 days, 6 months or 1 year; a sirtuin-modulating compound may be more stable in solution than resveratrol by at least a factor of about 50%, 2 fold, 5 fold, 10 fold, 30 fold, 50 fold or 100 fold; a sirtuin-modulating compound may promote deacetylation of the DNA repair factor Ku70; a sirtuin-modulating compound may promote deacetylation of RelA/p65; a compound may increase general turnover rates and enhance the sensitivity of cells to TNF-induced apoptosis.

In certain embodiments, a sirtuin-modulating compound does not have any substantial ability to inhibit a histone deacetylase (HDACs) class I, a HDAC class II, or HDACs I and II, at concentrations (e.g., in vivo) effective for modulating the deacetylase activity of the sirtuin. For instance, in preferred embodiments the sirtuin-modulating compound is a sirtuin-activating compound and is chosen to have an EC₅₀ for activating sirtuin deacetylase activity that is at least 5 fold less than the EC₅₀ for inhibition of an HDAC I and/or HDAC II, and even more preferably at least 10 fold, 100 fold or even 1000 fold less. Methods for assaying HDAC I and/or HDAC II activity are well known in the art and kits to perform such assays may be purchased commercially. See e.g., BioVision, Inc. (Mountain View, Calif.; world wide web at biovision.com) and Thomas Scientific (Swedesboro, N.J.; world wide web at tomassci.com).

In certain embodiments, a sirtuin-modulating compound does not have any substantial ability to modulate sirtuin homologs. In one embodiment, an activator of a human sirtuin protein may not have any substantial ability to activate a sirtuin protein from lower eukaryotes, particularly yeast or human pathogens, at concentrations (e.g., in vivo) effective for activating the deacetylase activity of human sirtuin. For example, a sirtuin-activating compound may be chosen to have an EC₅₀ for activating a human sirtuin, such as SIRT1 and/or SIRT3, deacetylase activity that is at least 5 fold less than the EC₅₀ for activating a yeast sirtuin, such as Sir2 (such as Candida, S. cerevisiae, etc.), and even more preferably at least 10 fold, 100 fold or even 1000 fold less. In another embodiment, an inhibitor of a sirtuin protein from lower eukaryotes, particularly yeast or human pathogens, does not have any substantial ability to inhibit a sirtuin protein from humans at concentrations (e.g., in vivo) effective for inhibiting the deacetylase activity of a sirtuin protein from a lower eukaryote. For example, a sirtuin-inhibiting compound may be chosen to have an IC₅₀ for inhibiting a human sirtuin, such as SIRT1 and/or SIRT3, deacetylase activity that is at least 5 fold less than the IC₅₀ for inhibiting a yeast sirtuin, such as Sir2 (such as Candida, S. cerevisiae, etc.), and even more preferably at least 10 fold, 100 fold or even 1000 fold less.

In certain embodiments, a sirtuin-modulating compound may have the ability to modulate one or more sirtuin protein homologs, such as, for example, one or more of human SIRT1, SIRT2, SIRT3, SIRT4, SIRT5, SIRT6, or SIRT7. In one embodiment, a sirtuin-modulating compound has the ability to modulate both a SIRT1 and a SIRT3 protein.

In other embodiments, a SIRT1 modulator does not have any substantial ability to modulate other sirtuin protein homologs, such as, for example, one or more of human SIRT2, SIRT3, SIRT4, SIRT5, SIRT6, or SIRT7, at concentrations (e.g., in vivo) effective for modulating the deacetylase activity of human SIRT1. For example, a sirtuin-modulating compound may be chosen to have an ED₅₀ for modulating human SIRT1 deacetylase activity that is at least 5 fold less than the ED₅₀ for modulating one or more of human SIRT2, SIRT3, SIRT4, SIRT5, SIRT6, or SIRT7, and even more preferably at least 10 fold, 100 fold or even 1000 fold less. In one embodiment, a SIRT1 modulator does not have any substantial ability to modulate a SIRT3 protein.

In other embodiments, a SIRT3 modulator does not have any substantial ability to modulate other sirtuin protein homologs, such as, for example, one or more of human SIRT1, SIRT2, SIRT4, SIRT5, SIRT6, or SIRT7, at concentrations (e.g., in vivo) effective for modulating the deacetylase activity of human SIRT3. For example, a sirtuin-modulating compound may be chosen to have an ED₅₀ for modulating human SIRT3 deacetylase activity that is at least 5 fold less than the ED₅₀ for modulating one or more of human SIRT1, SIRT2, SIRT4, SIRT5, SIRT6, or SIRT7, and even more preferably at least 10 fold, 100 fold or even 1000 fold less. In one embodiment, a SIRT3 modulator does not have any substantial ability to modulate a SIRT1 protein.

In certain embodiments, a sirtuin-modulating compound may have a binding affinity for a sirtuin protein of about 10⁻⁹M, 10⁻¹⁰ M, 10⁻¹¹M, 10⁻¹²M or less. A sirtuin-modulating compound may reduce (activator) or increase (inhibitor) the apparent Km of a sirtuin protein for its substrate or NAD⁺ (or other cofactor) by a factor of at least about 2, 3, 4, 5, 10, 20, 30, 50 or 100. Km values can be determined using mass spectrometry assays that are well known in the art. Preferred activating compounds reduce the Km of a sirtuin for its substrate or cofactor to a greater extent than caused by resveratrol at a similar concentration or reduce the Km of a sirtuin for its substrate or cofactor similar to that caused by resveratrol at a lower concentration. A sirtuin-modulating compound may increase the Vmax of a sirtuin protein by a factor of at least about 2, 3, 4, 5, 10, 20, 30, 50 or 100. A sirtuin-modulating compound may have an ED50 for modulating the deacetylase activity of a SIRT1 and/or SIRT3 protein of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A sirtuin-modulating compound may modulate the deacetylase activity of a SIRT1 and/or SIRT3 protein by a factor of at least about 5, 10, 20, 30, 50, or 100, as measured in a cellular assay or in a cell based assay. A sirtuin-activating compound may cause at least about 10%, 30%, 50%, 80%, 2 fold, 5 fold, 10 fold, 50 fold or 100 fold greater induction of the deacetylase activity of a sirtuin protein relative to the same concentration of resveratrol. A sirtuin-modulating compound may have an ED50 for modulating SIRT5 that is at least about 10 fold, 20 fold, 30 fold, 50 fold greater than that for modulating SIRT1 and/or SIRT3.

SIRT1 inhibitors also include RNA inhibitory molecules (RNAi) as described in US 2007/0185049, which is hereby incorporated by reference in its entirety, and as described elsewhere herein. Sirtuin inhibitors also include those disclosed in Grozinger et al., J. Biol. Chem. 42:38837-43 (2001), which is hereby incorporated by reference in its entirety. Sirtuin inhibitors include the compounds A3, sirtinol, and M15 described therein.

A “high dose” of a sirtuin activating compound refers to a quantity of a sirtuin activator having a sirtuin activating effect equal to or greater than the sirtuin activating effect of 18 mg/kg resveratrol (e.g., in humans). In certain embodiments, a high dose of a sirtuin activating compound refers to a quantity of a sirtuin activator having a sirtuin activating effect equal to or greater than the sirtuin activating effect of 18 mg/kg of resveratrol which is administered (i) orally, (ii) released from a sustained release form over 6 to 48 hours, and/or (iii) for an equivalent amount of time. In certain embodiments, a high dose of a sirtuin activating compound refers to a quantity of a sirtuin activator having a sirtuin activating effect equal to or greater than the sirtuin activating effect of at least about 20, 25, 30, 35, 40, 50, 60, 75, 100, 150 mg/kg, or more, or resveratrol.

“Sirtuin activating effect” refers to the level or extent of one or more therapeutic effects obtained upon administration of a high dose of a sirtuin activating compound. Therapeutic effects include, for example, (i) preventing or inhibiting weight gain upon consuming a diet having an increased fat and/or calorie content without an increase in activity, heart rate, and/or blood pressure; and/or (ii) improved blood glucose levels. Such therapeutic effects include, for example, the therapeutic effects illustrated in the Examples.

“Sirtuin protein” refers to a member of the sirtuin deacetylase protein family or preferably to the Sir2 family, which include yeast Sir2 (GenBank Accession No. P53685), C. elegans Sir-2.1 (GenBank Accession No. NP501912), and human SIRT1 (GenBank Accession No. NM012238 and NP036370 (or AF083106)) and SIRT2 (GenBank Accession No. NM030593 and AF083107) proteins. Other family members include the four additional yeast Sir2-like genes termed “HST genes” (homologues of Sir2) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

“SIRT1 protein” refers to a member of the sir2 family of sirtuin deacetylases. In one embodiment, a SIRT1 protein includes yeast Sir2 (GenBank Accession No. P53685), C. elegans Sir-2.1 (GenBank Accession No. NP501912), human SIRT1 (GenBank Accession No. NM012238 and NP036370 (or AF083106)), human SIRT2 (GenBank Accession No. NM012237, NM030593, NP036369, NP085096, and AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP036370, NP501912, NP085096, NP036369, and P53685. SIRT1 proteins include polypeptides comprising all or a portion of the amino acid sequence set forth in GenBank Accession Nos. NP036370, NP501912, NP085096, NP036369, and P53685; the amino acid sequence set forth in GenBank Accession Nos. NP036370, NP501912, NP085096, NP036369, and P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP036370, NP501912, NP085096, NP036369, and P53685 and functional fragments thereof. Polypeptides of the invention also include homologs (e.g., orthologs and paralogs), variants, or fragments, of GenBank Accession Nos. NP036370, NP501912, NP085096, NP036369, and P53685.

“Biologically active portion of a sirtuin” refers to a portion of a sirtuin protein having a biological activity, such as the ability to deacetylate. Biologically active portions of sirtuins may comprise the core domain of sirtuins. For example, amino acids 62-293 of the SIRT1 protein sequence, which are encoded by nucleotides 237 to 932 of the SIRT1 nucleic acid sequence, encompass the NAD⁺ binding domain and the substrate binding domain. Therefore, this region is sometimes referred to as the core domain. Other biologically active portions of SIRT1, also sometimes referred to as core domains, include about amino acids 261 to 447 of the SIRT1 protein sequence, which are encoded by nucleotides 834 to 1394 of the SIRT1 nucleic acid sequence; about amino acids 242 to 493 of the SIRT1 protein sequence, which are encoded by nucleotides 777 to 1532 of the SIRT1 nucleic acid sequence; or about amino acids 254 to 495 of the SIRT1 protein sequence, which are encoded by nucleotides 813 to 1538 of the SIRT1 nucleic acid sequence.

Nampt is a nicotinamide phosphribosyltransferase enzyme (NAMPRT; E.C.2.4.2.12) that metabolizes nicotinamide. The human gene encoding Nampt is also referred to as pre-B-cell colony enhancing factor 1(PBEF1) and visfatin and exists as two isoforms (Samal et al., Mol Cell Biol 1994, 14:1431; Rongwaux et al., Eur J Immunol 2002, 32:3225; Fukuhara et al., Science 2005, 307:426-30; U.S. Pat. Nos. 5,874,399 and 6,844,163). The sequence of isoform a is available under GenBank Accession numbers NM_{—}005746, NP_{—}005737 and U02020 and the sequence of isoform b is available under GenBank Accession numbers NM_{—}182790, NP_{—}877591 and BC020691. The sequence of a genomic clone of human NAMPRT is provided in GenBank Accession No. AC007032. The structure of the human gene is described in Ognjanovic et al., J Mol Endocrinol 2001, 26:107.

The terms “comprise” and “comprising” are used in the inclusive, open sense, meaning that additional elements may be included.

The term “including” is used to mean “including but not limited to”. “Including” and “including but not limited to” are used interchangeably.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The terms “polynucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement.

A “patient”, “subject”, “individual” or “host” refers to either a human or a non-human animal. Non-human animals include farm animals (e.g., cows, horses, pigs, sheep, goats) and companion animals (e.g., dogs, cats).

The term “substantially homologous” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

The term “modulation” is art-recognized and refers to up regulation (i.e., activation or stimulation), down regulation (i.e., inhibition or suppression) of a response, or the two in combination or apart.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “mammal” is known in the art, and exemplary mammals include humans, primates, bovines, porcines, canines, felines, and rodents (e.g., mice and rats).

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

The term “pharmaceutical” refers to any compound having a pharmacological effect. For example, the term pharmaceutical encompasses natural compounds as well as nonnatural compounds that have a pharmacological effect.

The term “pharmaceutically-acceptable salts” is art-recognized and refers to the relatively non-toxic, inorganic and organic acid addition salts of compounds, as well as solvates, co-crystals, polymorphs and the like of the salts, including, for example, those contained in compositions described herein.

The term “pharmaceutically acceptable carrier” is art-recognized and refers to a pharmaceutically-acceptable material, composition or vehicle, such as a liquid or solid filler, diluent, excipient, solvent or encapsulating material. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion.

“Transcriptional regulatory sequence” is a generic term used throughout the specification to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

“Treating” a condition or disease refers to curing as well as ameliorating at least one symptom of the condition or disease.

The term “therapeutic agent” is art-recognized and refers to any compound that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “effective amount” means that amount of such a substance that produces some desired local or systemic effect at a reasonable benefit/risk ratio applicable to any treatment. The effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect on metabolic disorders or diabetes or complications thereof, at a reasonable benefit/risk ratio applicable to such treatment.

The term “synthetic” is art-recognized and refers to production by in vitro chemical or enzymatic synthesis.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

### Downregulation of SIRT1 or Nampt

SIRT1 or Nampt expression may be inhibited by the use of any method which results in decreased transcription of the gene encoding SIRT1 or Nampt. In some embodiments, RNAi technology can be used to inhibit or downregulate the expression of SIRT1 or Nampt by decreasing transcription of the gene encoding SIRT1 or Nampt. RNA interference or “RNAi” is a term initially coined by Fire and co-workers to describe the observation that double-stranded RNA (dsRNA) can block gene expression when it is introduced into worms (Fire et al. (1998) Nature 391, 806-811). “RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. See for example U.S. patent application Ser. Nos: 20030153519A1; 20030167490A1; and U.S. Pat. Nos. 6,506,559; 6,573,099, which are herein incorporated by reference in their entirety.

Isolated RNA molecules specific to SIRT1 mRNA or Nampt mRNA, which mediate RNAi, are antagonists useful in the method of the present invention. In one embodiment, the RNA interfering agents used in the methods of the invention. Exemplary SIRT1 RNA interfering agents are described in US 2007/0185049 and US 2007/0160586, which are incorporated by reference herein for that teaching.

Other strategies for delivery of the RNA interfering agents, e.g., the siRNAs or shRNAs of used in the methods of the invention, may also be employed, such as, for example, delivery by a vector, e.g., a plasmid or viral vector, e.g., a lentiviral vector. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

The RNA interfering agents, e.g., siRNAs, may also be administered in combination with other pharmaceutical agents.

An “RNA interfering agent” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), and small molecules which interfere with or inhibit expression of a target gene by RNA interference (RNAi). The target gene of the present invention is the gene encoding SIRT1.

As used herein, “inhibition of target gene expression” includes any decrease in expression or protein activity or level of the target gene or protein encoded by the target gene. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, 5, or 6 nucleotides. The length of the overhang is independent between the two strands, i.e., the length of the overhang on one strand is not dependent on the length of the overhang on the second strand. In one embodiment, the siRNA can inhibit SIRT1 by transcriptional silencing. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA Apr; 9(4):493-501, incorporated by reference herein).

A siRNA may be substantially homologous to the target SIRT1 gene or genomic sequence, or a fragment thereof. As used herein, the term “homologous” is defined as being substantially identical, sufficiently complementary, or similar to the target mRNA, or a fragment thereof, to effect RNA interference of the target. In addition to native RNA molecules, RNAs suitable for inhibiting or interfering with the expression of a target sequence include RNA derivatives and analogs. siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues may be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

SIRT1 expression may also be inhibited in vivo by the use of any method which results in decreased transcription of the gene encoding SIRT1. One embodiment uses antisense technology. Gene expression can be controlled through triple-helix formation or antisense DNA or RNA, both of which methods are based on binding of a polynucleotide to DNA or RNA. An antisense nucleic acid molecule which is complementary to a nucleic acid molecule encoding SIRT1 can be designed based upon the isolated nucleic acid molecules encoding SIRT1 by means known to those in the art.

Design and Preparation of siRNA Molecules

Synthetic siRNA molecules, including shRNA molecules, for use in the methods described herein, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are routinely synthesized and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell. 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule of the present invention can be selected from a given target gene sequence, e.g., an apoptosis-related gene or a cytokine, beginning from about 25 to 50 nucleotides, from about 50 to 75 nucleotides, or from about 75 to 100 nucleotides downstream of the start codon. Nucleotide sequences may contain 5′ or 3′ UTRs and regions nearby the start codon. One method of designing a siRNA molecule of the present invention involves identifying the 23 nucleotide sequence motif AA(N19)TT (where N can be any nucleotide) and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search may be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23 nucleotide sequence motif. The use of symmetric 3′ TT overhangs may be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis companies such as Oligoengine®, may also be used to select siRNA sequences against EST libraries to ensure that only one gene is targeted.

### Delivery of RNA Interfering Agents

Methods of delivering RNA interfering agents, e.g., an siRNA of the present invention, or vectors containing an RNA interfering agent, e.g., an siRNA of the present invention, to the target cells for uptake include injection of a composition containing the RNA interfering agent, e.g., an siRNA, or directly contacting the cell, with a composition comprising an RNA interfering agent, e.g., an siRNA.

A viral-mediated delivery mechanism may also be employed to deliver siRNAs to cells in vitro and in vivo as described in Xia, H. et al. (2002) Nat Biotechnol 20(10): 1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Other methods of introducing siRNA molecules of the present invention to target cells, e.g., tumor cells, include a variety of art-recognized techniques including, but not limited to, calcium phosphate or calcium chloride co-precipitation, DEAE-dextran-mediated transfection, lipofection, or electroporation as well as a number of commercially available transfection kits (e.g., OLIGOFECTAMINE® Reagent from Invitrogen) (see, e.g. Sui, G. et al. (2002) Proc. Natl. Acad. Sci. USA 99:5515-5520; Calegari, F. et al. (2002) Proc. Natl. Acad. Sci., USA Oct. 21, 2002; J-M Jacque, K. Triques and M. Stevenson (2002) Nature 418:435-437; and Elbashir, S. M et al. (2001) supra). Suitable methods for transfecting a target cell, e.g., a tubular cell of the kidney or a cardiac cell can also be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals. The efficiency of transfection may depend on a number of factors, including the cell type, the passage number, the confluency of the cells as well as the time and the manner of formation of siRNA- or shRNA-liposome complexes (e.g., inversion versus vortexing). These factors can be assessed and adjusted without undue experimentation by one with ordinary skill in the art.

The RNA interfering agents, e.g., the siRNAs or shRNAs of the invention, may be introduced along with components that perform one or more of the following activities: enhance uptake of the RNA interfering agents, e.g., siRNA, by the cell, inhibit annealing of single strands, stabilize single strands, or otherwise facilitate delivery to the target cell and increase inhibition of the target gene, SIRT1.

### Administration

The pharmacological agents used in the methods of the invention are preferably sterile and contain an effective amount of one or more agents for producing the desired response in a unit of weight or volume suitable for administration to a subject. The doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent may be adjusted by the individual physician or veterinarian, particularly in the event of any complication. A therapeutically effective amount typically varies from 0.01 mg/kg to about 1000 mg/kg, preferably from about 0.1 mg/kg to about 500 mg/kg, and most preferably from about 0.2 mg/kg to about 250 mg/kg, in one or more dose administrations daily, for one or more days.

Pharmacological agents associated with the invention and optionally other therapeutics may be administered per se or in the form of a pharmaceutically acceptable salt.

Various modes of administration are known to those of ordinary skill in the art which effectively deliver the pharmacological agents of the invention to a desired tissue, cell, or bodily fluid. The administration methods are discussed elsewhere in the application. The invention is not limited by the particular modes of administration disclosed herein. Standard references in the art (e.g., Remington's Pharmaceutical Sciences, 20th Edition, Lippincott, Williams and Wilkins, Baltimore Md., 2001) provide modes of administration and formulations for delivery of various pharmaceutical preparations and formulations in pharmaceutical carriers. Other protocols which are useful for the administration of pharmacological agents of the invention will be known to one of ordinary skill in the art, in which the dose amount, schedule of administration, sites of administration, mode of administration and the like vary from those presented herein.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A pharmacological agent or composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride, chlorobutanol, parabens and thimerosal.

The pharmaceutical compositions may conveniently be presented in unit dosage form and may be prepared by any of the methods well known in the art of pharmacy. All methods include the step of bringing the active agent into association with a carrier, which constitutes one or more accessory ingredients. In general, the compositions are prepared by uniformly and intimately bringing the active compound into association with a liquid carrier, a finely divided solid carrier, or both, and then, if necessary, shaping the product.

The pharmacological agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle (e.g., saline, buffer, or sterile pyrogen-free water) before use.

Compositions suitable for oral administration may be presented as discrete units, such as capsules, tablets, pills, lozenges, each containing a predetermined amount of the active compound. Other compositions include suspensions in aqueous liquids or non-aqueous liquids such as a syrup, elixir, an emulsion, or a gel.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, sorbitol or cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the agent or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration can also be as a powder, lightly compressed plugs or even as tablets. The therapeutic can be prepared by compression.

Colorants and flavoring agents may all be included. For example, agents may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of an agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery. Agents can be delivered to the lungs of a mammal while inhaling and traverse across the lung epithelial lining to the blood stream. Reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (al-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-γ and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of a given agent. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise an agent dissolved in water at a concentration of about 0.1 to 25 mg of biologically active agent per mL of solution. The formulation may also include a buffer and a simple sugar (e.g., for stabilization and regulation of osmotic pressure). The nebulizer formulation may also contain a surfactant, to reduce or prevent surface induced aggregation of the agent caused by atomization of the solution in forming the aerosol.

Formulations for use with a metered-dose inhaler device will generally comprise a finely divided powder containing the agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing an agent and may also include a bulking agent, such as lactose, sorbitol, sucrose, or mannitol in amounts which facilitate dispersal of the powder from the device, e.g., 50 to 90% by weight of the formulation. The agent should most advantageously be prepared in particulate form with an average particle size of less than 10 mm (or microns), most preferably 0.5 to 5 mm, for most effective delivery to the distal lung.

Nasal (or intranasal) delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic agent(s), may be provided in particles. Particles as used herein means nano or micro particles (or in some instances larger) which can consist in whole or in part of therapeutic agent(s) described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain therapeutic agents described herein in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The blood-brain barrier (BBB) excludes many highly hydrophilic compounds. The therapeutic agents may be delivered to the brain using a formulation capable of delivering a therapeutic agent across the blood brain barrier. One obstacle to delivering therapeutics to the brain is the physiology and structure of the brain. The blood-brain barrier is made up of specialized capillaries lined with a single layer of endothelial cells. The region between cells are sealed with a tight junction, so the only access to the brain from the blood is through the endothelial cells. The barrier allows only certain substances, such as lipophilic molecules through and keeps other harmful compounds and pathogens out. Thus, lipophilic carriers are useful for delivering non-lipohilic compounds to the brain. For instance, DHA, a fatty acid naturally occurring in the human brain has been found to be useful for delivering drugs covalently attached thereto to the brain (Such as those described in U.S. Pat. No. 6,407,137). U.S. Pat. No. 5,525,727 describes a dihydropyridine pyridinium salt carrier redox system for the specific and sustained delivery of drug species to the brain. U.S. Pat. No. 5,618,803 describes targeted drug delivery with phosphonate derivatives. U.S. Pat. No. 7,119,074 describes amphiphilic prodrugs of a therapeutic compound conjugated to an PEG-oligomer/polymer for delivering the compound across the blood brain barrier. The compounds described herein may be modified by covalent attachment to a lipophilic carrier or co-formulation with a lipophilic carrier. The therapeutic compounds also can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V. V. Ranade, 1989, J. Clin. Pharmacol. 29:685). Others are known to those of skill in the art.

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

For topical administration to the eye, nasal membranes, mucous membranes or to the skin, the therapeutic agents may be formulated as ointments, creams or lotions, or as a transdermal patch or intraocular insert or iontophoresis. For example, ointments and creams can be formulated with an aqueous or oily base alone or together with suitable thickening and/or gelling agents. Lotions can be formulated with an aqueous or oily base and, typically, further include one or more emulsifying agents, stabilizing agents, dispersing agents, suspending agents, thickening agents, or coloring agents. (See, e.g., U.S. Pat. No. 5,563,153, entitled “Sterile Topical Anesthetic Gel”, issued to Mueller, D., et al., for a description of a pharmaceutically acceptable gel-based topical carrier.)

In general, the therapeutic agent is present in a topical formulation in an amount ranging from about 0.01% to about 30.0% by weight, based upon the total weight of the composition. Preferably, the agent is present in an amount ranging from about 0.5 to about 30% by weight and, most preferably, the agent is present in an amount ranging from about 0.5 to about 10% by weight. In one embodiment, the compositions of the invention comprise a gel mixture to maximize contact with the surface of the localized pain and minimize the volume and dosage necessary to alleviate the localized pain. GELFOAM® (a methylcellulose-based gel manufactured by Upjohn Corporation) is a preferred pharmaceutically acceptable topical carrier. Other pharmaceutically acceptable carriers include iontophoresis for transdermal drug delivery.

The invention also contemplates the use of kits. In some aspects of the invention, the kit can include a pharmaceutical preparation vial, a pharmaceutical preparation diluent vial, and one or more therapeutic agents. In some embodiments the kit contains agents for diagnostic purposes such as an antibody or multiple antibodies. The vial containing the diluent for the pharmaceutical preparation is optional. The diluent vial contains a diluent such as physiological saline for diluting what could be a concentrated solution or lyophilized powder of a therapeutic agent. The instructions can include instructions for mixing a particular amount of the diluent with a particular amount of the concentrated pharmaceutical preparation, whereby a final formulation for injection or infusion is prepared. The instructions may include instructions for treating a subject with an effective amount of a therapeutic agent. The instructions may include instructions for diagnosing a patient, characterizing a patient's risk for a given disease, or evaluating the effectiveness of a given therapy for a patient. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

Also provided are methods an assays for identifying modulators of the circadian rhythm. Such methods include contacting a cell that expresses SIRT1, CLOCK and BMAL with a candidate molecule, and determining the level, activity or acetylation state of a biomarker indicating circadian rhythm activity. When the level or acetylation state of the biomarker in the cell that has been contacted with the candidate molecule differs from a reference or control level of the level or acetylation state of the biomarker, then the candidate molecule is a modulator of circadian rhythm.

A variety of biomarkers can be used in such methods and assays. Exemplary biomarkers include SIRT1 expression CLOCK acetylase activity, BMAL1 acetylation state, such as the state of acetylation of lysine 537, and PER2 acetylation state.

A wide variety of assays to identify candidate molecules that modulate the circadian rhythm can be used in accordance with the aspects of the invention, including, labeled in vitro protein-protein binding assays, electrophoretic mobility shift assays, immunoassays, cell-based assays such as two- or three-hybrid screens, expression assays, etc. The assay mixture comprises a candidate molecule(s). Typically, a plurality of assay mixtures are run in parallel with different agent concentrations to obtain a different response to the various concentrations. Typically, one of these concentrations serves as a negative control, i.e., at zero concentration of candidate molecule or at a concentration of candidate molecule below the limits of assay detection.

Candidate molecules encompass numerous chemical classes, although typically they are organic compounds. In some embodiments, the candidate molecules are small organic compounds, i.e., those having a molecular weight of more than 50 yet less than about 2500, preferably less than about 1000 and, more preferably, less than about 500. Candidate molecules comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate molecules can comprise cyclic carbon or heterocyclic structure and/or aromatic or polyaromatic structures substituted with one or more of the above-identified functional groups. Candidate molecules also can be biomolecules such as peptides, saccharides, fatty acids, sterols, isoprenoids, purines, pyrimidines, derivatives or structural analogs of the above, or combinations thereof and the like. Where the candidate molecule is a nucleic acid molecule, the agent typically is a DNA or RNA molecule, although modified nucleic acid molecules as defined herein are also contemplated.

It is contemplated that cell-based assays as described herein can be performed using cell samples and/or cultured cells. Cells include cells that transformed to express SIRT1 protein, CLOCK protein and/or BMAL protein, and cells treated to modulate (e.g. inhibit or enhance) the level and/or activity of a SIRT1 protein, CLOCK protein and/or BMAL protein or a complex of these proteins.

Candidate molecules are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the assay mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

An assay may be used to identify candidate molecules that modulate circadian rhythm by modulating interaction of SIRT1/CLOCK or SIRT1/CLOCK/BMAL or by modulating the activity or expression of one or more of these proteins. In general, the mixture of the foregoing assay materials is incubated under conditions whereby, but for the presence of the candidate molecule(s), SIRT1/CLOCK or SIRT1/CLOCK/BMAL interact, i.e., bind and form a complex. It will be understood that a candidate molecule that is identified as a modulator may be identified as increasing, or reducing SIRT1/CLOCK or SIRT1/CLOCK/BMAL interaction. A reduction in SIRT1/CLOCK or SIRT1/CLOCK/BMAL interaction need not (but can) be the absence of SIRT1/CLOCK or SIRT1/CLOCK/BMAL interaction, but may be a lower level of SIRT1/CLOCK or SIRT1/CLOCK/BMAL interaction. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the interaction of SIRT1/CLOCK or SIRT1/CLOCK/BMAL or the modulation of the activity or expression of one or more of these proteins is detected by any convenient method available to the user.

The invention also provides agents such as antibodies that specifically bind to BMAL1 polypeptide acetylated at lysine 537. Such agents can be used in methods of the invention including for diagnosis. Such binding agents also can be used, for example, in screening assays to detect the presence or absence of acetylated BMAL1 polypeptides and can be used in quantitative binding assays to determine levels of acetylated BMAL1 polypeptides in biological samples and cells.

In preferred embodiments, the binding polypeptide is an antibody or antibody fragment, more preferably, an Fab or F(ab)₂ fragment of an antibody. Typically, the fragment includes a CDR3 region that is selective for the acetylated BMAL polypeptide. Any of the various types of antibodies can be used for this purpose, including polyclonal antibodies, monoclonal antibodies, humanized antibodies, and chimeric antibodies.

Thus, the invention provides antibodies that bind to acetylated BMAL polypeptides. Such antibodies can be used in screening assays to detect the presence or absence of an acetylated BMAL polypeptide and in purification protocols to isolate such polypeptides. Likewise, such antibodies can be used to selectively target drugs, toxins or other molecules (including detectable diagnostic molecules) to cells which express acetylated BMAL polypeptides.

The antibodies of the present invention thus are prepared by any of a variety of methods, including administering a protein, fragments of a protein, cells expressing the protein or fragments thereof and the like to an animal to induce polyclonal antibodies. The present invention also provides methods of producing monoclonal antibodies to the acetylated BMAL1 polypeptide as described herein. The production of monoclonal antibodies is performed according to techniques well known in the art. As detailed herein, such antibodies may be used for example to identify tissues expressing protein or to purify protein. Antibodies also may be coupled to specific labeling agents or imaging agents, including, but not limited to a molecule preferably selected from the group consisting of fluorescent, enzyme, radioactive, metallic, biotin, chemiluminescent, bioluminescent, chromophore, or colored, etc. In some aspects of the invention, a label may be a combination of the foregoing molecule types.

Significantly, as is well-known in the art, only a small portion of an antibody molecule, the paratope, is involved in the binding of the antibody to its epitope (see, in general, Clark, W. R., 1986, The Experimental Foundations of Modern Immunology, Wiley & Sons, Inc., New York; Roitt, I., 1991, Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762, and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)2, Fab, Fv, and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

The present invention is further illustrated by the following examples which should not be construed as limiting in any way. The contents of all cited references (including literature references, issued patents, published patent applications, and GenBank accession numbers as cited throughout this application) are hereby expressly incorporated by reference.

### EXAMPLES

### Example 1

### SIRT1 Deacetylase Activity is Circadian

### Experimental Procedures

### Animals

Male BALB/c mice and liver-specific Sirt1−/− mice were housed under 12 hr light/12 hr dark (LD) cycles over 2 weeks. All protocols using animals were approved by the to Institutional Animal Care and Use Committee of the University of California, Irvine.

### Plasmids

FLAG-tagged and Myc-tagged plasmids have been described (Doi et al., 2006; Travnickova-Bendova et al., 2002). Full and truncated mouse Clock ORFs were amplified by PCR and cloned in pG4 MpolyII vector. Mouse Clock(D19) was amplified by PCR from c/c MEFs. Mouse SIRT1 ORF was subcloned into pcDNA3 with a FLAG epitope sequence at the 50 end. hSIRT1-Flag/pcDNA3.1, hSIRT2-Flag/pcDNA3.1, and hSIRT3-Flag/pcDNA3.1 were kind gifts of E. Verdin. Flag-hSIRT1/pECE, Flag-hSIRT1(H363Y)/pECE, hSIRT1-HA/pECE, and hSIRT1(H363Y)-HA/pECE were kind gifts of A. Brunet.

### Antibodies

Antibodies against acetyl-histone H3, histone H4, and SIRT1 were from Millipore, antibodies against CLOCK and rabbit IgG from Santa Cruz Biotechnology, and antibodies against Flag (M2) and b-actin from Sigma. Antibodies against BMAL1 and Myc were described (Cardone et al., 2005). Polyclonal acetyl-lysine 537 BMAL1antibody was generated by immunizing rabbits with KLH-conjugates of the peptide (SEQ ID NO:16) NH₂-ASSPGG[acetyl-K]KILN-(mouse BMAL1).

### Cell Culture

MEFs were generated from WT or homozygous Sirt1−/− sibling mice and cultured in DMEM (4.5 g/l glucose) supplemented with 7.5% newborn bovine serum, 2.5% FBS, and antibiotics. JEG3 cells were grown in Basal Medium Eagle supplemented with 10% FBS and antibiotics.

Preparation of Cell Extracts and Nuclear Extracts from Cultured Cell Lines

Cells were washed twice with phosphate-buffered saline (PBS) and lysed in RIPA buffer (50 mM Tris/HCL [pH 8.0], 150 mM NaCl, 5 mM EDTA, 15 mM MgCl2, 1% NP40, 13 protease inhibitor cocktail [Roche], 1 mM DTT, 1 mM trichostatin A [TSA], 10 mM NAM, 10 mM NaF, 1 mM PMSF). For nuclear extracts (Andrews and Faller, 1991), after washing cells with cold PBS, cells were lysed with hypotonic buffer (10 mM HEPES-KOH [pH 7.9], 1.5 mM MgCl2, 10 mM KCl, 13 protease inhibitor cocktail [Roche], 1 mM DTT, 1 mM TSA, 10 mM NAM, 10 mM NaF, 1 mM PMSF). Following a brief centrifugation, pellet was resuspended in hypertonic buffer (20 mM HEPES-KOH [pH 7.9], 25% glycerol, 420 mM NaCl 1.5 mM MgCl2, 0.2 mMEDTA, 13 protease inhibitor cocktail [Roche], 1 mM DTT, 1 mM TSA, 10 mM NAM, 10 mM NaF, 1 mM PMSF). Supernatants were recovered as nuclear extracts.

### ChIP Assays

Conventional ChIP assay was used for histones from MEFs (Yamamoto et al., 2004). For nonhistone proteins, dual crosslinking ChIP assay (Nowak et al., 2005) was used with slight modifications. After serum shock, cells were washed three times with room temperature PBS, then PBS/1 mM MgCl2 was added. Disuccinimidyl Glutalate (DSG) was added to a final concentration of 2 mM for crosslinking. After 45 min at room temperature, formaldehyde was added to a final concentration of 1% (v/v) and cells incubated for 15 min. After dual crosslinking, glycine was added to a final concentration of 0.1 M and incubated for 10 min to quench formaldehyde out. After harvesting, cells were lysed in 500 ml ice-cold cell lysis buffer (50 mM Tris/HCl [pH 8.0], 85 mM KCl, 0.5% NP40, 1 mM PMSF, 13 protease inhibitor cocktail [Roche]) for 10 min on ice. Nuclei were precipitated by centrifugation (3000 g for 5 min), resuspended in 600 ml ice-cold RIPA buffer (50 mM Tris/HCl [pH 8.0], 150 mM NaCl, 1 mM EDTA [pH 8.0], 1% Triton X-100, 0.1% SDS, 0.1% sodium deoxycholate, 1 mM PMSF, 13 protease inhibitor cocktail [Roche]), and incubated on ice for 30 min. Sonication was done to obtain DNA fragments 100-600 by in length.

### Quantitative Real-Time RT-PCR

Each quantitative real-time RT-PCR was performed using the Chromo4 real time detection system (BIO-RAD). The PCR primers for mDbp mRNA, mPer2 mRNA, mCry 1 mRNA, 18S rRNA, Dbp UP, Dbp E-box, Dbp 30R, and mSIRT1 mRNA were described (Ripperger and Schibler, 2006; Rodgers et al., 2005; Yamamoto et al., 2004). PCR primers for Dbp TSS were designed using Real-Time PCR Primer Design (https://www.genscript.com/ssl-bin/app/primer), and the sequences are available upon request. For a 20 ml PCR, 50 ng of cDNA template was mixed with the primers to final concentrations of 200 nM and 10 ml of iQ SYBR Green Supermix (BIO-RAD), respectively. The reaction was first incubated at 95° C. for 3 min, followed by 40 cycles at 95° C. for 30 s and 60° C. for 1 min.

### RNase Protection Assays

RNA extractions were done using TRIzol (GIBCO BRL). RNase protection assays (RPAs) were performed as described (Pando et al., 2002). The riboprobes were generated using an in vitro transcription kit (Promega). Data were quantified using a phosphorimager.

### Recombinant Proteins, [³⁵S] Labeling, and GST Pulldown Assay

GST-fused recombinant proteins were expressed in E. coli BL21. Recombinant proteins were lysed by CelLytic B Cell Lysis Reagent (Sigma) according to the manufacturer's protocol and purified by glutathione Sepharose 4B (Amersham). ³⁵S-methionine-labeled proteins were made in vitro using the TNT-T7 quick-coupled transcription-translation system (Promega). Twenty microliters of in vitro-translated ³⁵S-methionine-labeled proteins and 1 mg of GST-mSIRT1 or GST on glutathione Sepharose were added in a 1 ml binding buffer (50 mM Tris/HCl [pH 8.0], 150 mM NaCl, 1% NP-40), incubated overnight at 4° C. After washing sepharose with binding buffer three times, proteins were analyzed on SDS-PAGE.

### SIRT1 Deacetylation Assay

SIRT1 deacetylase activity was determined using a SIRT1 Fluorimetric Activity Assay/Drug Discovery Kit (AK-555; BIOMOL International) following the manufacturer's protocol. Extracts from serum-stimulated MEFs and liver from entrained mice lysed by RIPA buffer were used for measuring SIRT1 deacetylase activity. Complementation assays were performed by adding recombinant E. coli-generated SIRT1, and they included 1 U/reaction of SIRT1 protein and 25 mM of substrate (acetylated p53) in a 50 ml final volume. Endogenous SIRT1 from liver was obtained by immunoprecipitation and then incubated in deacetylase buffer with the substrate and 0.1 mM NAD⁺ for 1 hr at 37° C.

### Results

### SIRT1 Deacetylase Activity is Circadian

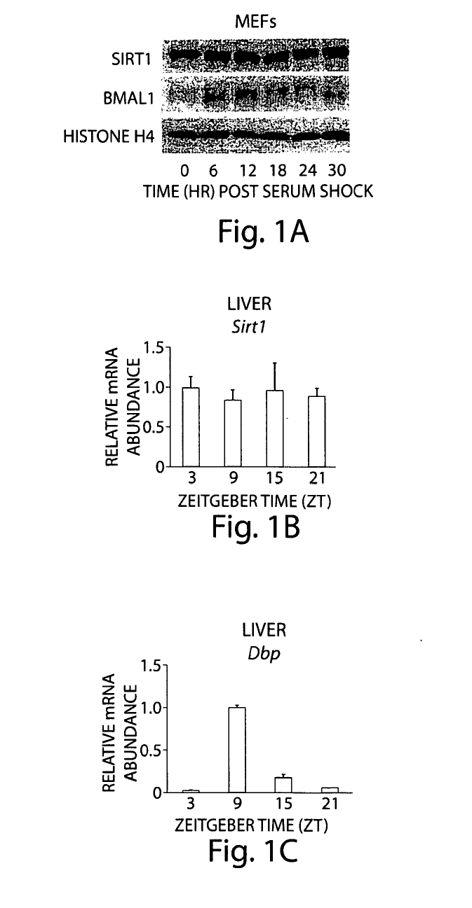

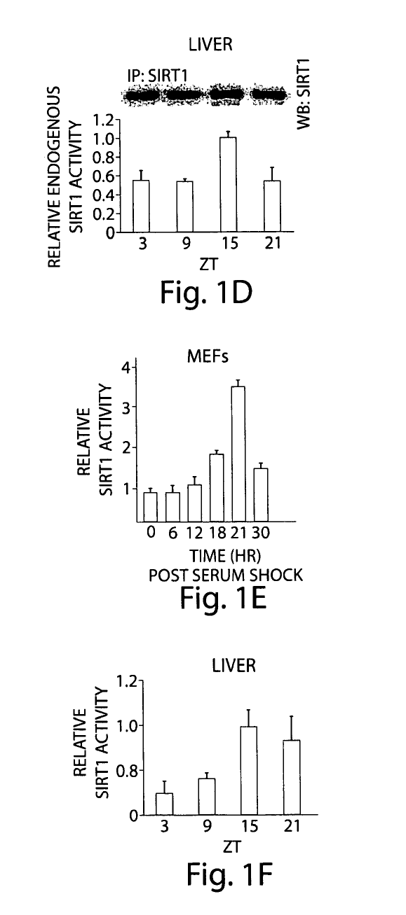

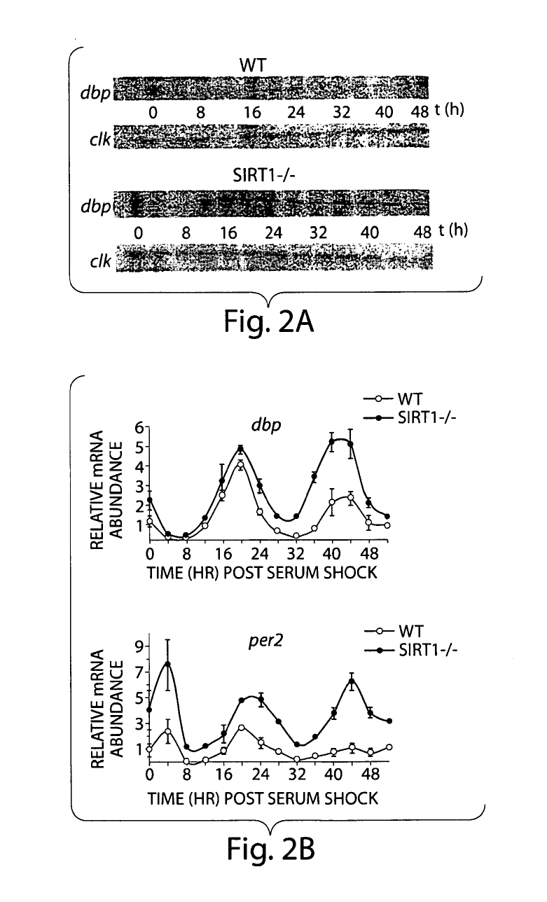

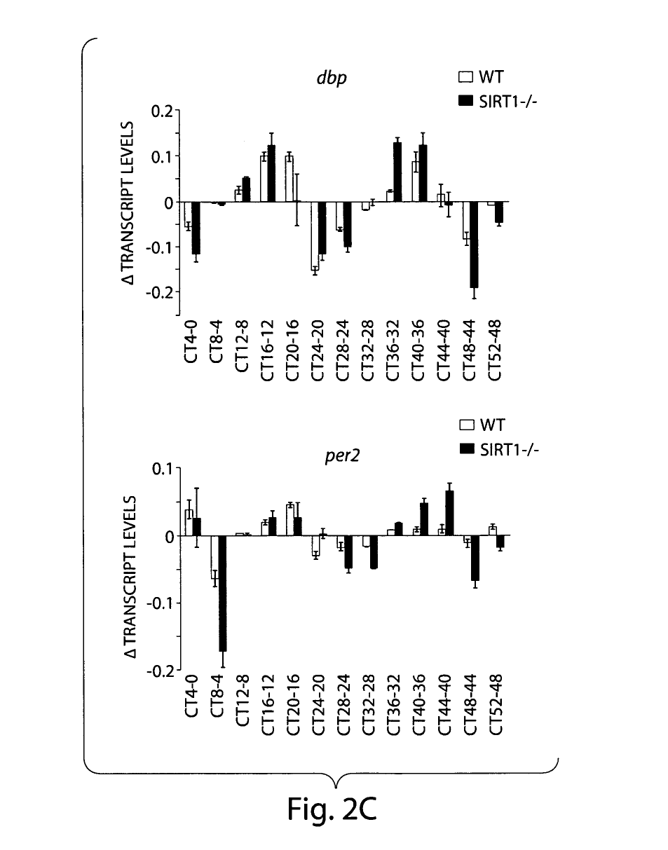

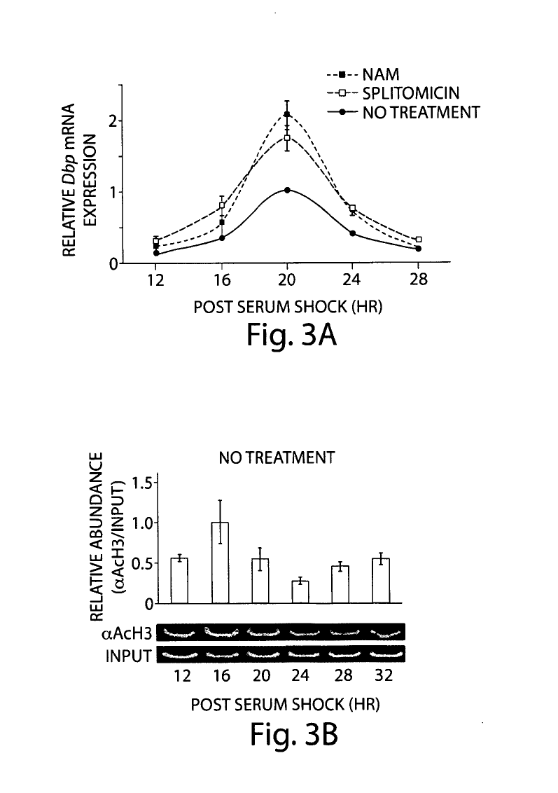

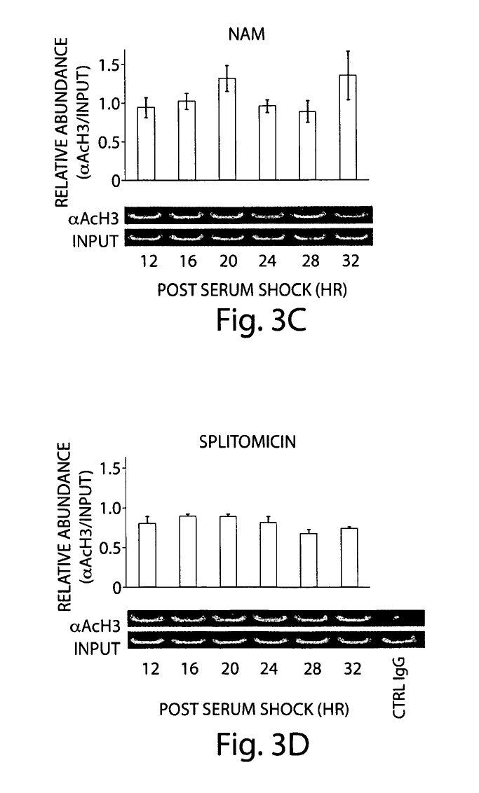

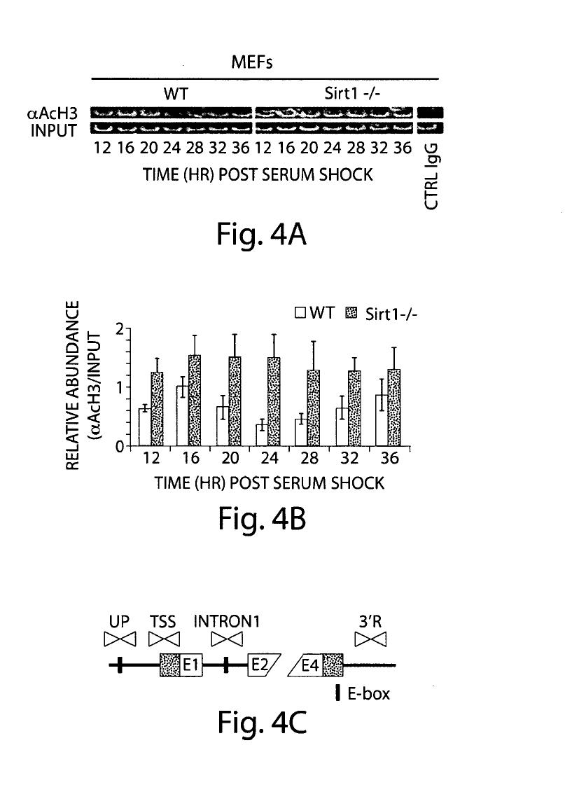

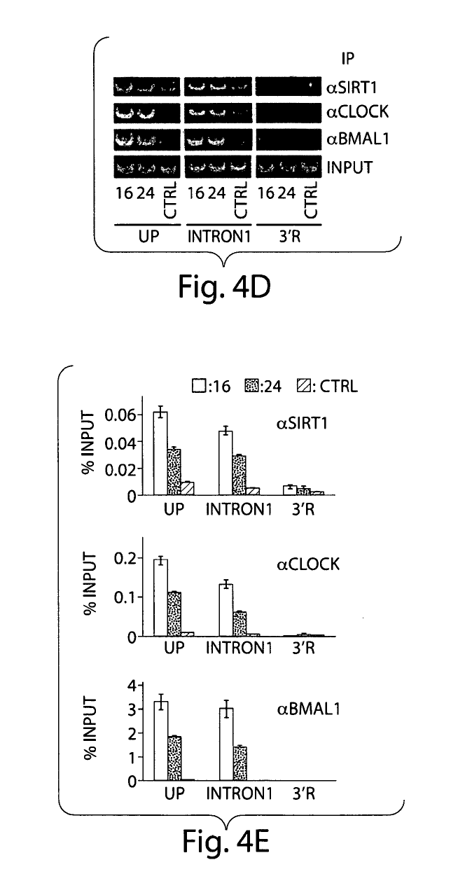

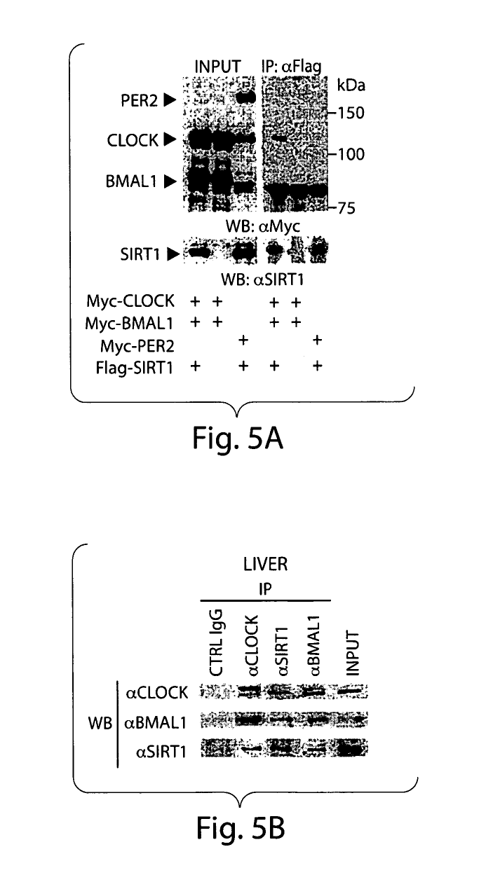

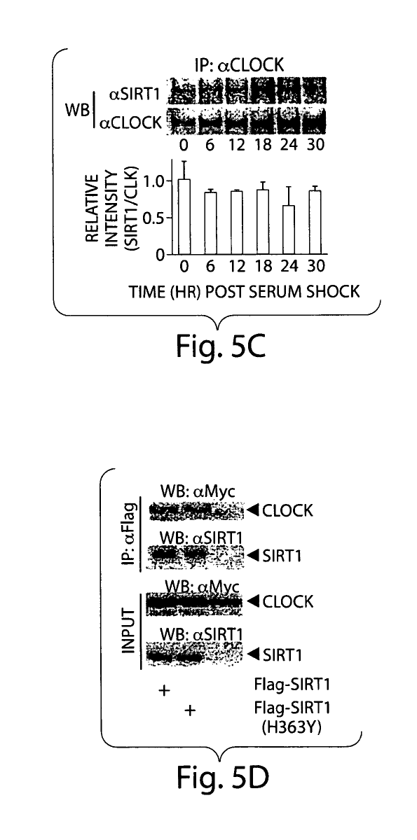

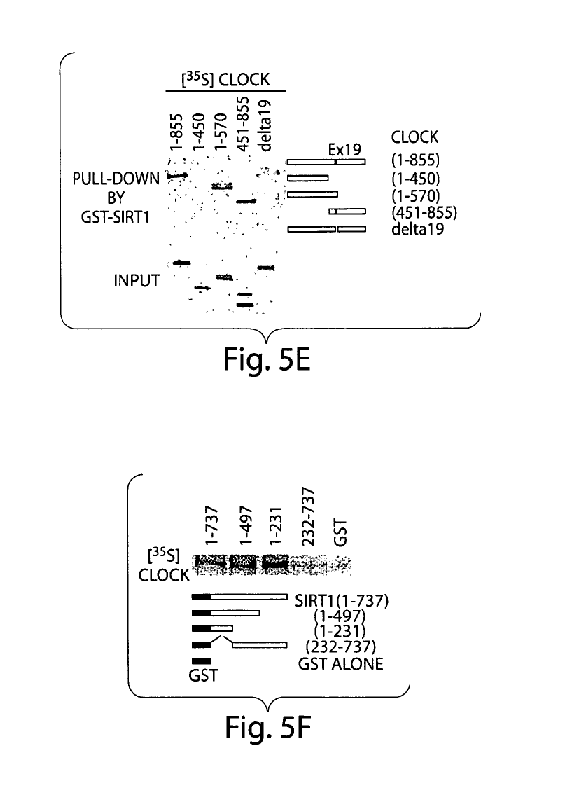

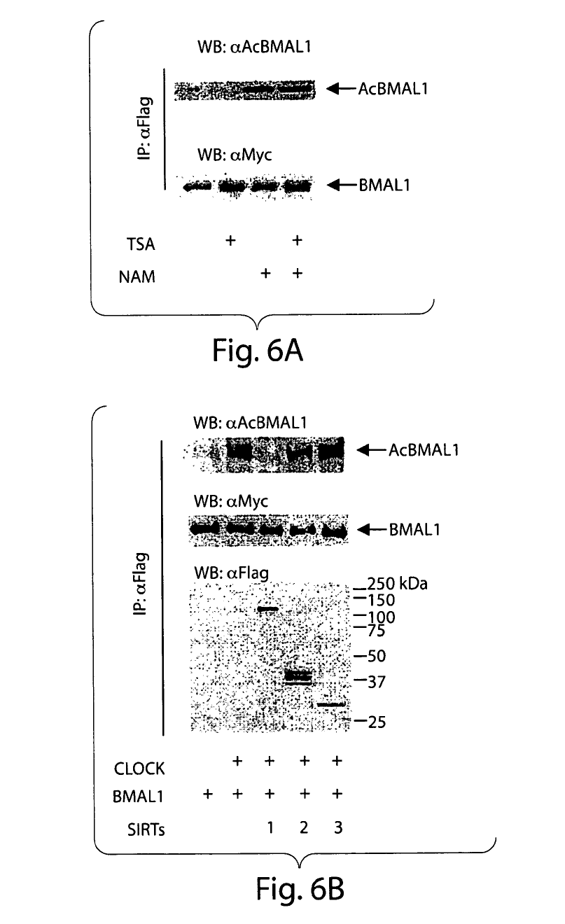

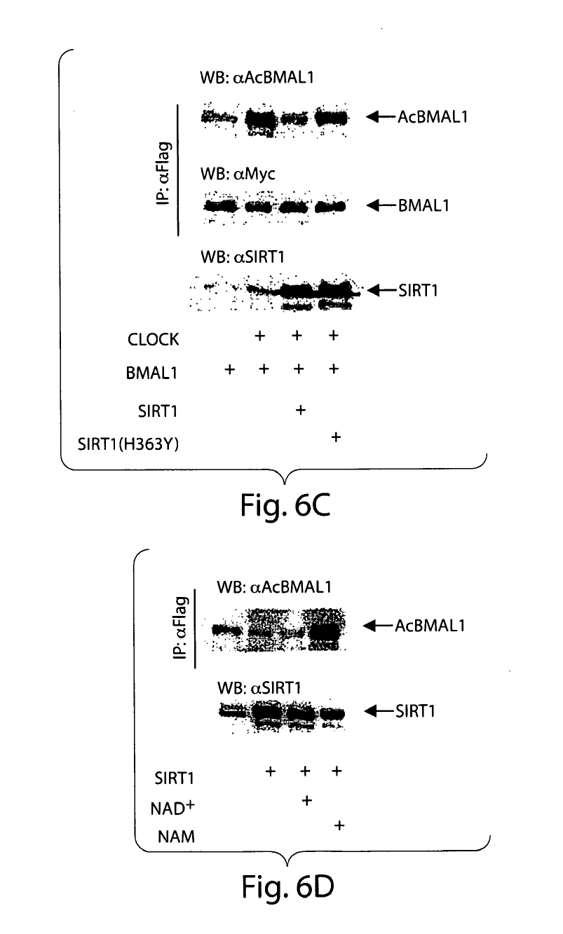

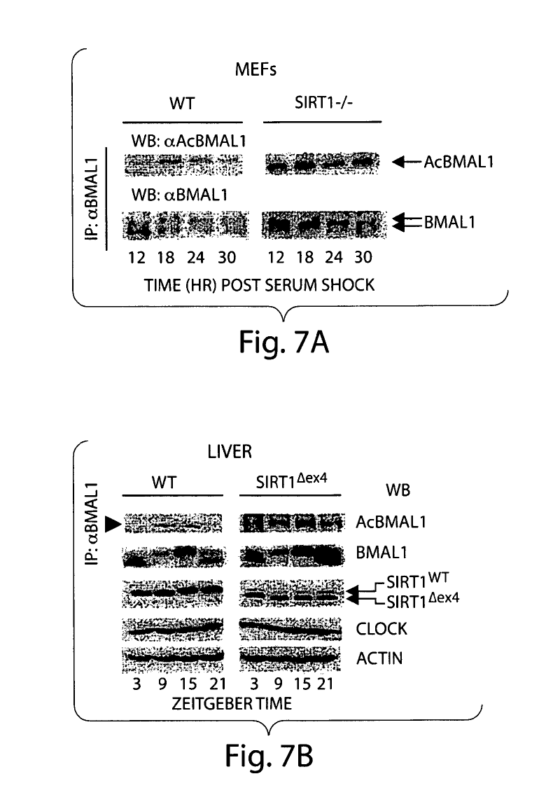

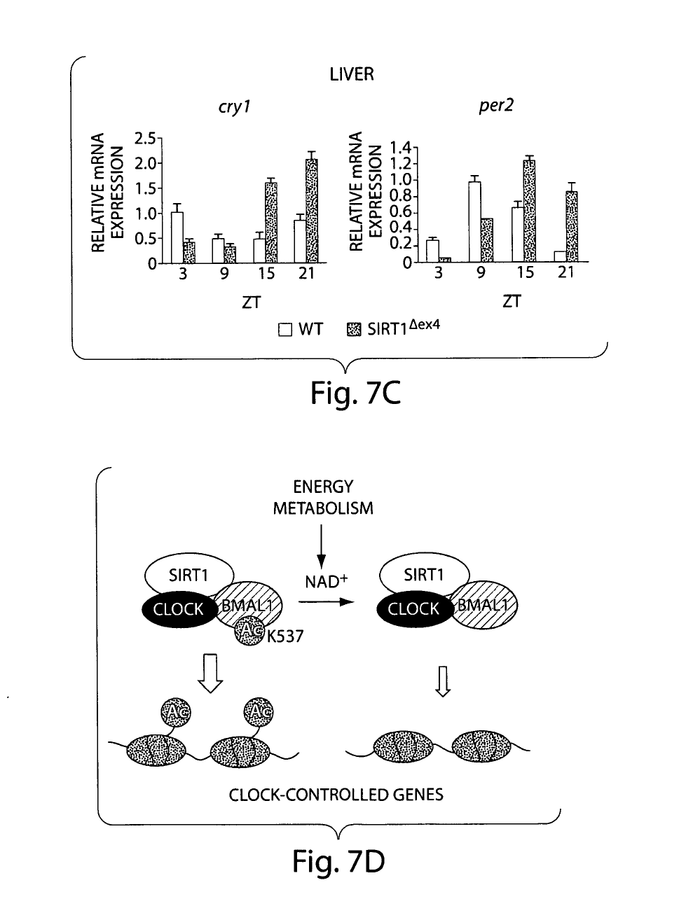

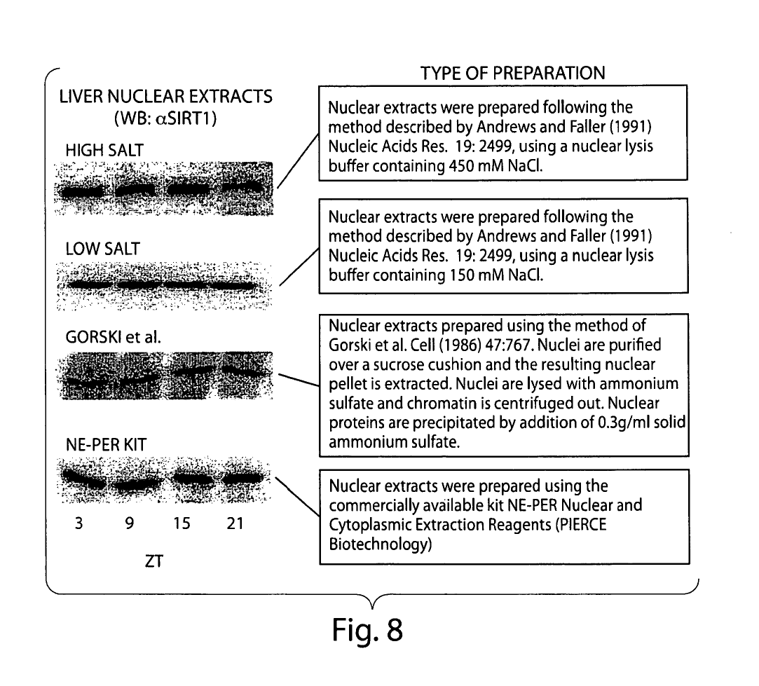

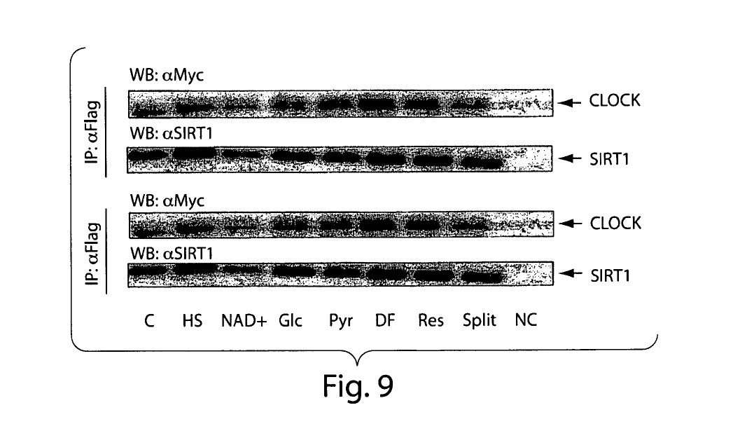

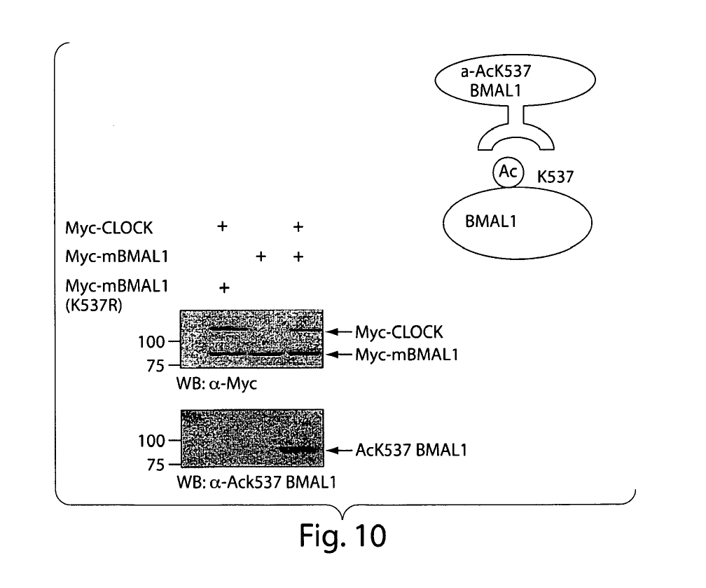

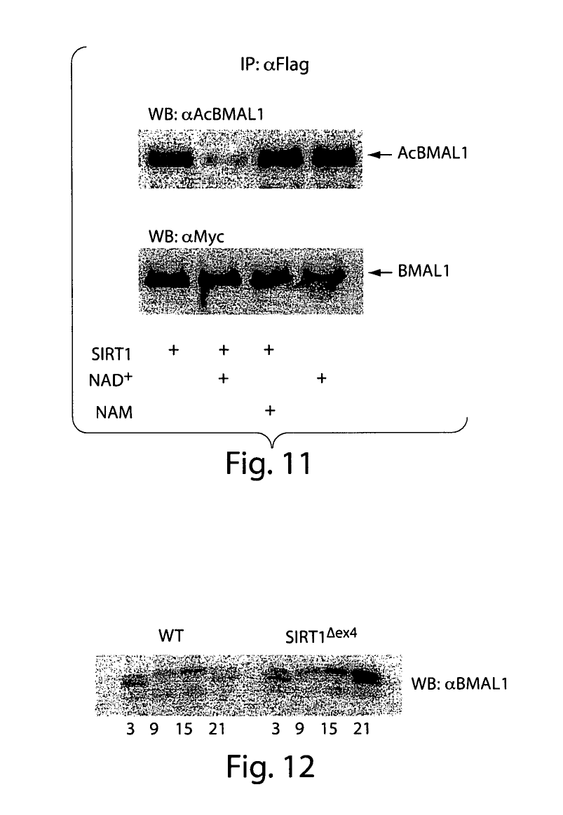

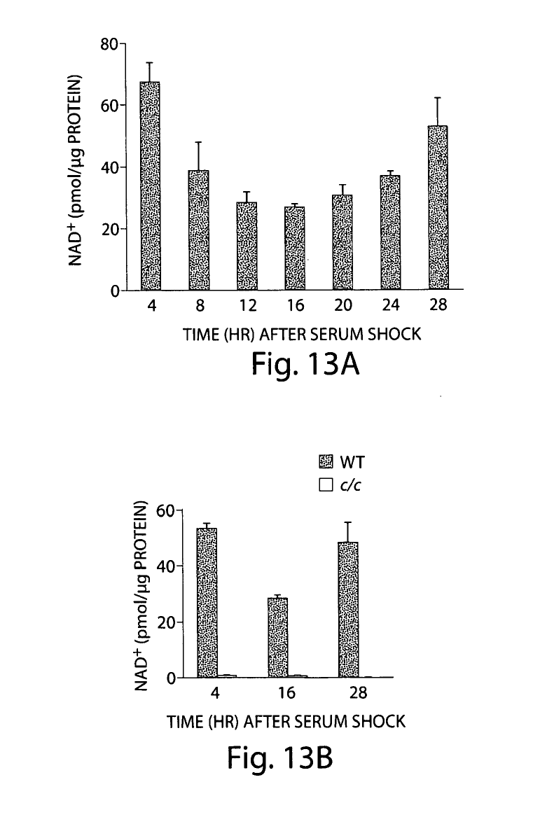

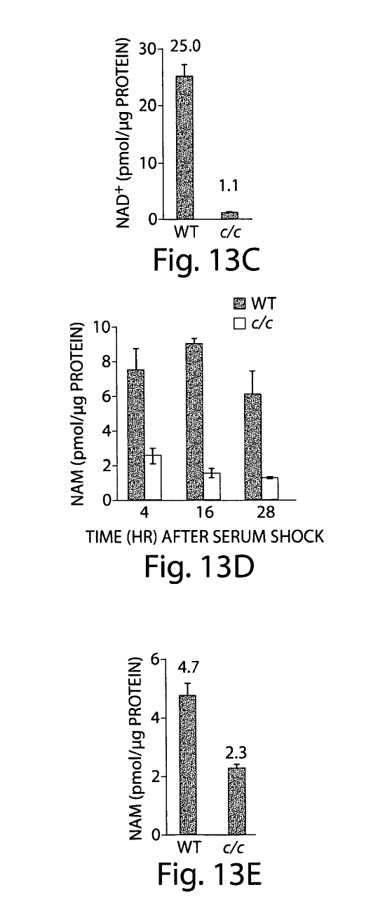

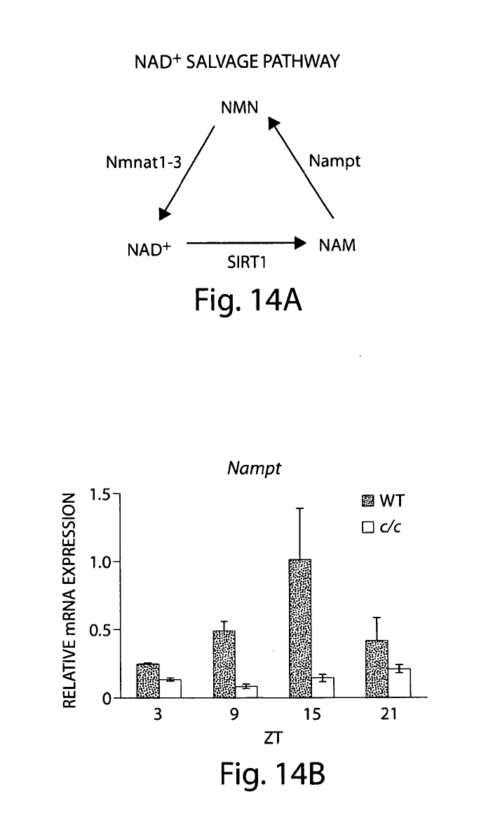

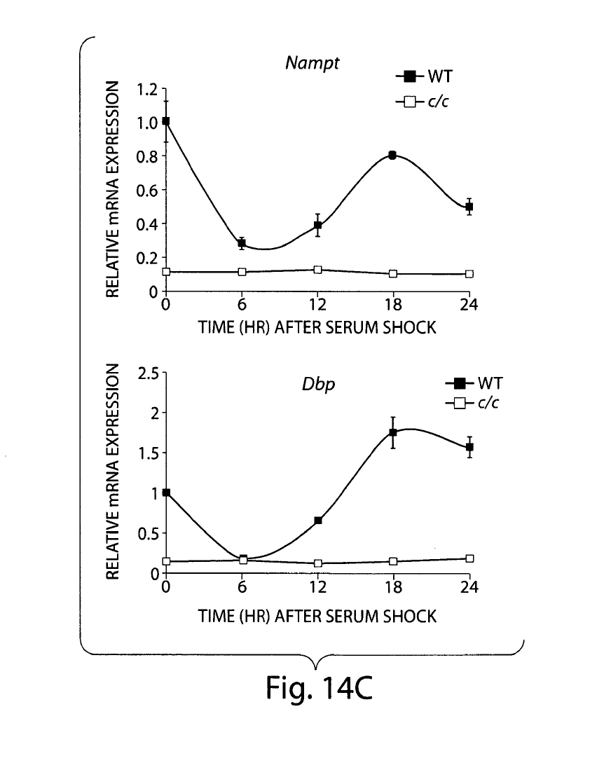

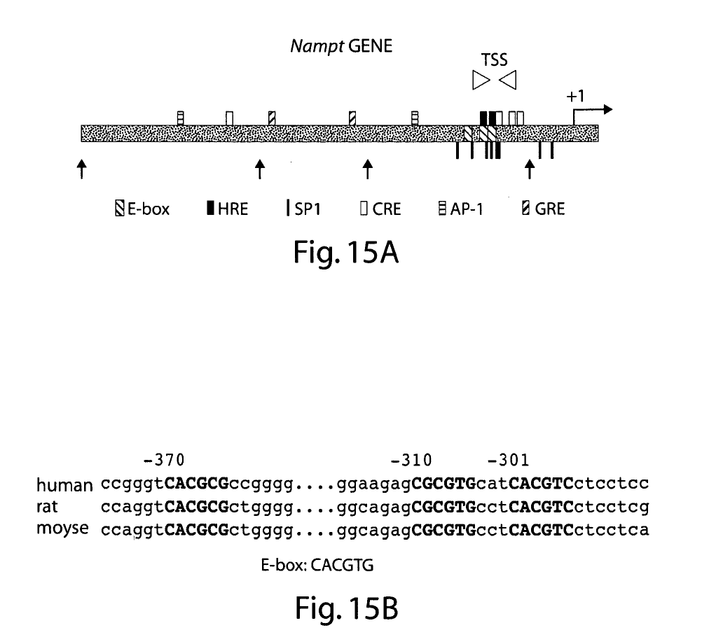

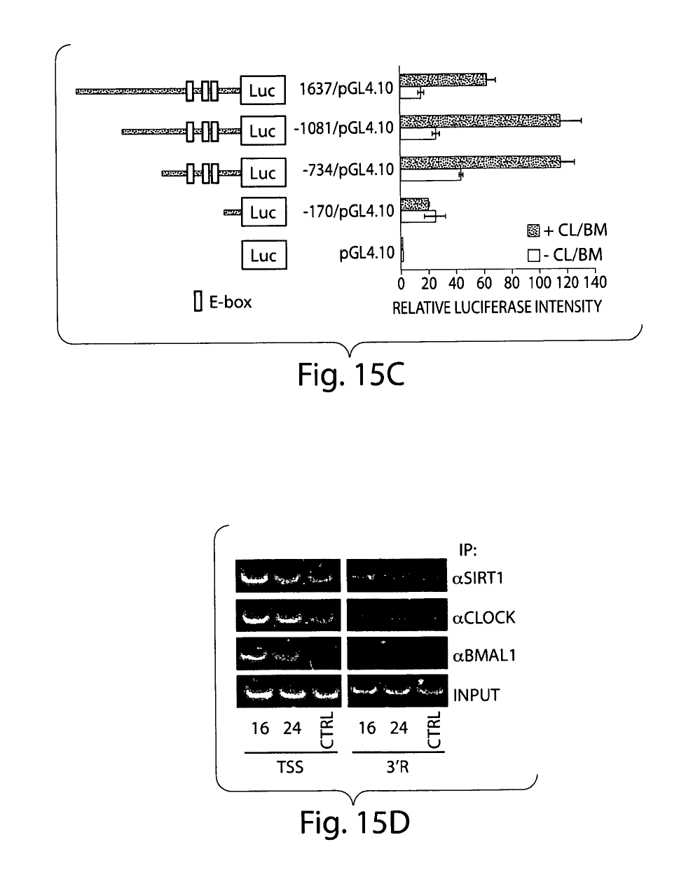

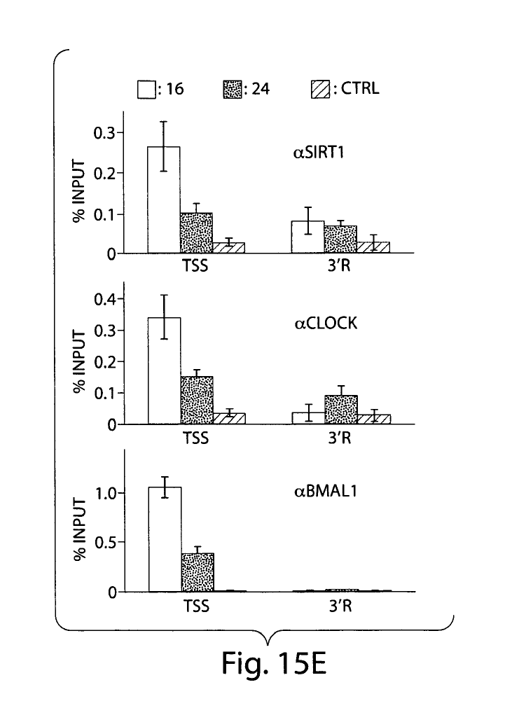

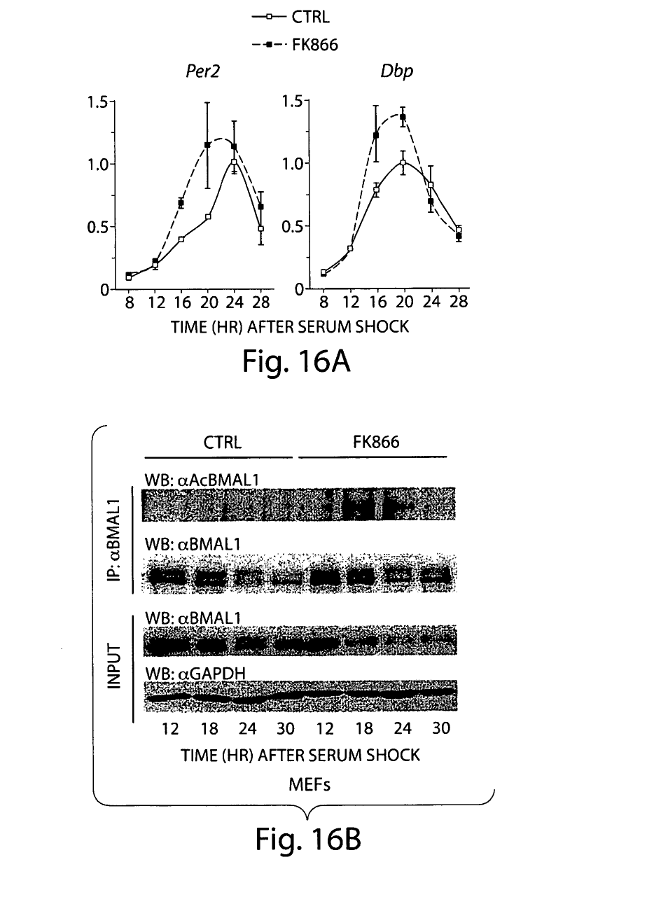

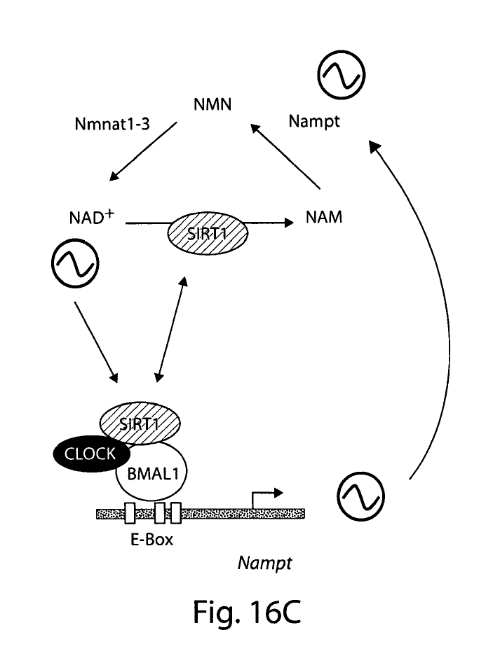

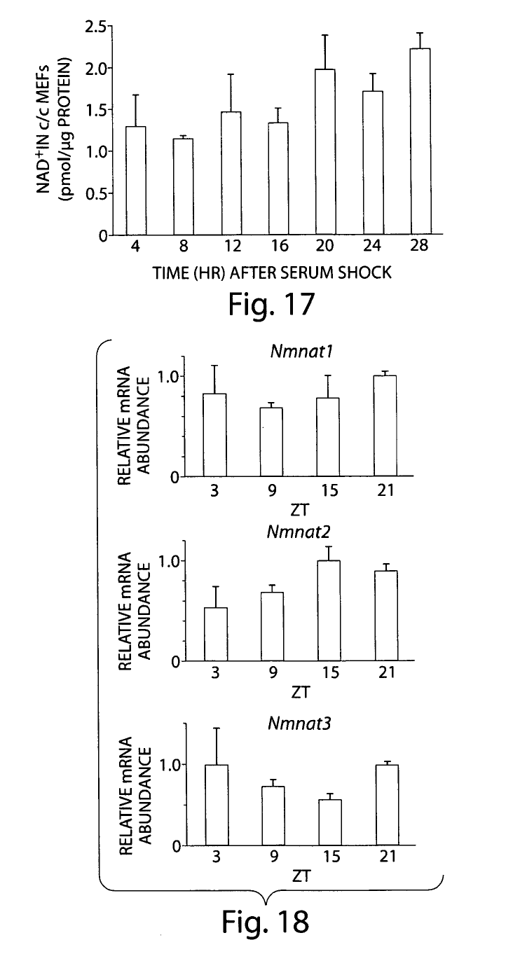

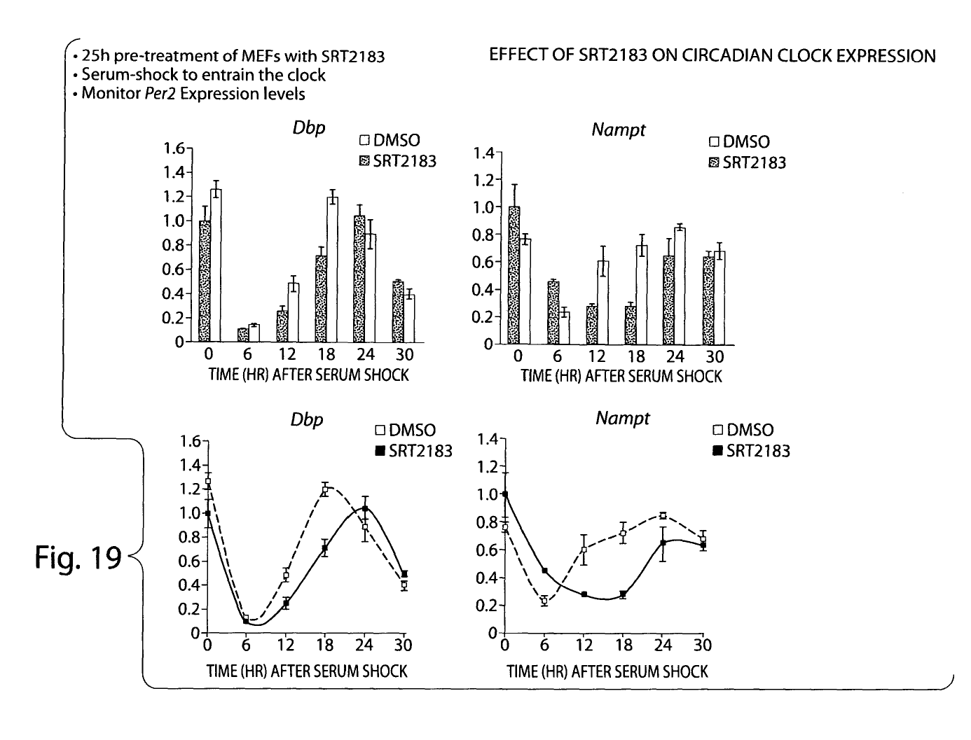

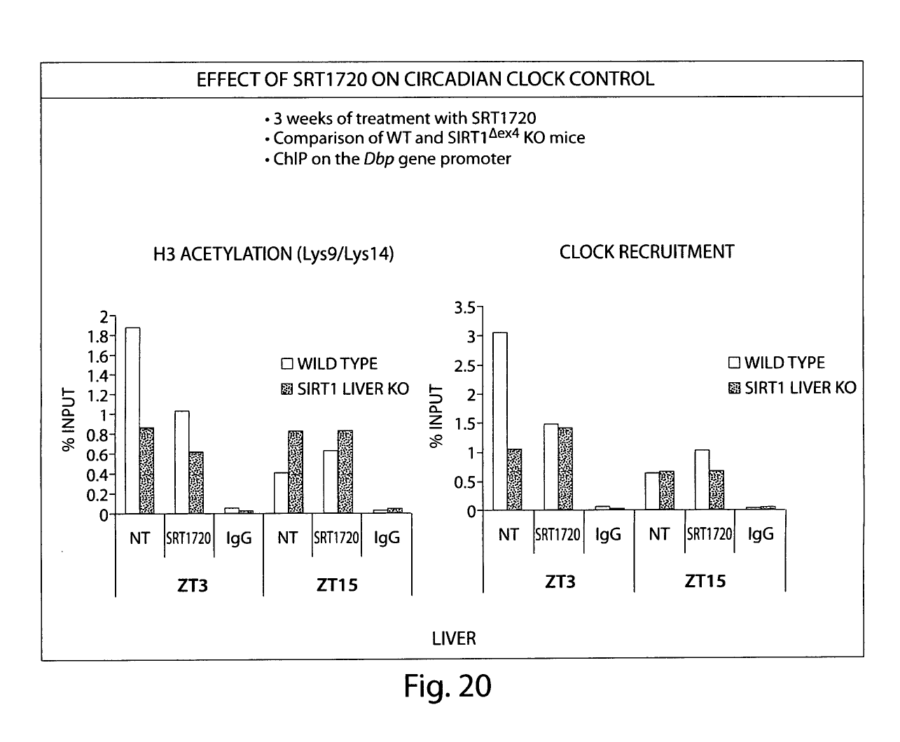

The CLOCK protein is one of the few core circadian regulators whose levels do not oscillate in most settings (Lee et al., 2001). Thus, we predicted that its HAT activity would oscillate in a circadian manner, thereby explaining the physiological remodeling of chromatin (Doi et al., 2006). An alternative scenario implicates a regulated HDAC, whose activity may function as rheostat of the HAT's function of CLOCK. To assess whether SIRT1 may be regulated in a circadian manner we prepared RNA and protein extracts from serum-stimulated cultured MEFs and from mouse liver at various Zeitgeber times (ZT). In both cases, the transcript and protein levels of SIRT1 remained constant, as determined using two anti-SIRT1-specific antibodies (FIG. 1A; see also later, FIG. 7B) and reverse transcription (RT)-PCR (FIGS. 1B and 1C, where dbp is shown as a control from the same RNA preparations). We have also determined the levels of SIRT1 in nuclear fractions prepared in various manners. Again SIRT1 protein levels showed either modest or no oscillation (FIG. 8). Thus, we turned to determining whether SIRT1 deacetylase activity may oscillate. We found that the endogenous liver SIRT1 obtained by immunoprecipitation from entrained mice, although constant in levels, displays circadian HDAC activity that peaks at ZT15 (FIG. 1D), a time that remarkably parallels the minimal transcriptional levels of various clock-controlled genes in the liver (such as dbp, FIG. 1C).

To confirm that equal levels of SIRT1 could still generate circadian HDAC function, we complemented cellular extracts with equal amounts of recombinant SIRT1 protein and acetylated p53 peptide as SIRT1 substrate (Luo et al., 2001; Vaziri et al., 2001). This assay likely reflects the intracellular relative concentrations of NAD⁺ and NAM, or of yet undefined circadian metabolites, whose ratio determines SIRT1 activity (Imai et al., 2000; Luo et al., 2001). Extracts from wild-type (WT) MEFs were prepared every 6 hr post-serum shock (FIG. 1E), and liver extracts were prepared at four different ZT from entrained mice (FIG. 1F). Also, under these conditions we found that SIRT1 deacetylase activity is rhythmic, peaking 24 hr post-serum shock in MEFs (FIG. 1E) and at ZT 15 in the liver (FIG. 1F). Importantly, the peak of SIRT1 deacetylase activity is consistent with the cyclic acetylation of histone H3 at promoters of clock-controlled genes; at 24 hr, this acetylation is at its lowest levels (see later, FIG. 4).

### SIRT1 Contributes to the Stringency of Circadian Gene Expression

The finding that SIRT1 activity is regulated in a circadian manner prompted us to investigate its role in clock gene expression and chromatin remodeling. MEFs generated from WT and Sirt1 null mice were serum-shocked, RNA was prepared at various times, and quantitative RNase protection assay was used to monitor dbp circadian gene transcription (FIG. 2A). The analysis reveals that genetic ablation of SIRT1 causes changes in circadian gene expression, including an overall increase in the transcription levels and a broadening of the oscillation cycles, with earlier onsets of increasing transcription and later decreases. Importantly, the expression of a non oscillating gene (clock) is not affected (FIG. 2A). These observations are consistent with SIRT1 having a role in controlling the stringency of circadian gene expression and being involved in the oscillatory silencing that periodically follows a transcriptional peak. These results were confirmed by quantitative RT-PCR and reproduced also on the Per2 gene (FIG. 2B). Finally, a point-by-point circadian analysis of the differential transcription of both dbp and per2 between WT and Sirt1−/− MEFs confirms that the lack of Sirt1 induces a significantly higher transcriptional efficacy at specific times that normally precede and follow each circadian expression peak (FIG. 2C).

The effect of Sirt1 genetic ablation on circadian gene expression was confirmed with pharmacological treatments using SIRT1 inhibitors (splitomicin and NAM) (FIG. 3A), which had no effect on the Sirt1−/− MEFs (not shown). Analysis of dbp expression levels in WT MEFs treated with either inhibitors revealed a pattern highly similar to the one obtained with the Sirt1 null cells, basically displaying a significantly broader phase and higher amplitude in the oscillation (FIG. 3A).

### SIRT1 Controls Circadian Histone Acetylation

The finding that CLOCK is a HAT revealed that chromatin remodeling is intimately connected to circadian physiology (Doi et al., 2006; Grimaldi et al., 2007). The remarkable effect of SIRT1 on clock gene expression (FIGS. 2 and 3) suggested that this effect may be mediated by changes in histone acetylation at specific sites. We analyzed the acetylation at Lys9 and Lys14 of histone H3 because we had previously found these to be preferential sites of CLOCK's HAT activity (Doi et al., 2006), and also because SIRT1 deacetylase function was described to be targeted to these lysines (Imai et al., 2000). We performed chromatin immunoprecipitation (ChIP) assays using WT MEFs stimulated by a serum shock. Acetylation of H3 at the Dbp transcription start site (TSS) follows a circadian profile (FIG. 3B and Ripperger and Schibler, 2006), presumably due to the concerted action of CLOCK and of an HDAC. Pharmacological treatment with the SIRT1 inhibitors (splitomicin and NAM) induced a loss in the circadian oscillation of acetylation, generating a noncyclic, high level of Lys9/Lys14 acetylation (FIGS. 3C and 3D).

Next, we extended this analysis to the MEFs from the Sirt1−/− mice. Again, histone H3 displays a robust cyclic acetylation at the Dbp promoter in WT MEFs, while genetic ablation of Sirt1 results in a constitutive, high acetylation at Lys9/Lys14 (FIGS. 4A and 4B). Thus, SIRT1 plays a critical role in maintaining a controlled rhythmically in histone acetylation, thereby contributing to circadian chromatin remodeling.

SIRT1 Is in a Chromatin Complex with CLOCK:BMAL1 on the Dbp Promoter

Based on the pattern of histone acetylation associated with circadian genes (FIGS. 3 and 4A), it is conceivable that CLOCK and SIRT1 converge in a coordinate manner to the same regulatory regions. Thus, we decided to test whether SIRT1 is recruited to E-box elements present in the regulatory region of CLOCK: BMAL1-controlled genes. To do so, we performed a dual crosslinking ChIP assay and analyzed two E-box elements within the Dbp gene. As predicted (Ripperger and Schibler, 2006), CLOCK and BMAL1 are recruited to E-box elements in the Dbp gene in a time-dependent manner (FIG. 4D). Importantly, we found that SIRT1 is jointly recruited to the same E-box elements in the Dbp gene. Furthermore, the presence of SIRT1 is temporally regulated and parallels the recruitment of the CLOCK:BMAL1 dimer (FIGS. 4D and 4E). Since SIRT1 is not a DNA-binding protein, we favor a scenario in which SIRT1 recruiting to a circadian promoter is mediated by the CLOCK:BMAL1 dimer. These results indicate that CLOCK:BMAL1 and SIRT1 coexist in a chromatin regulatory complex that operates on circadian promoters.

### Direct Interaction of SIRT1 and CLOCK

The coordinated recruiting of the CLOCK:BMAL1 dimer and SIRT1 to circadian gene promoters suggested that these regulators may physically interact. To test this possibility we coexpressed SIRT1 with CLOCK in cultured cells. In coimmunoprecipitation experiments we reveal that SIRT1 interacts with CLOCK but not with PER2 (FIG. 5A). Next, we wished to establish whether native, endogenous cellular SIRT1 interacts with CLOCK. Native SIRT1 can be coimmunoprecipitated with both CLOCK and BMAL1 in liver extracts, indicating that it interacts with the CLOCK:BMAL1 complex (FIG. 5B). It is unclear why BMAL1 in transfected cells does not seem to coimmunoprecipitate with the SIRT1-CLOCK complex (FIG. 5A), but the results on native proteins (FIG. 5B) suggest the requirement of some specific physiological conditions.

We also followed the SIRT1-CLOCK interaction during the circadian cycle (FIG. 5C). To do so, we coimmunoprecipitated SIRT1 from MEFs by using anti-CLOCK-specific antibody at various times after serum shock. While it would appear that the association undergoes some mild variations, after quantification of three different experiments we concluded that the SIRT1-CLOCK interaction is mostly stable during the circadian cycle (FIG. 5C). We have also found that the CLOCK-SIRT1 interaction is not significantly modulated by agents known or likely to influence SIRT1 function, including NAD⁺, pyruvate, resveratrol, splitomicin, desferroxamine, and glucose (FIG. 9).

Finally, the CLOCK-SIRT1 interaction does not appear to require the HDAC function. We used a SIRT1 mutant with a single amino acid substitution that diminishes the deacetylase activity (SIRT1-(H363Y); Vaziri et al., 2001). The mutated protein interacts with CLOCK with efficacy equivalent to that of normal SIRT1(FIG. 5D). To identify the protein regions involved in SIRT1-CLOCK association, we performed GST pulldown assays (FIGS. 5E and 5F). We found that the central region of CLOCK (aa 450-570) is necessary for interaction with SIRT1. Interestingly, this region contains the serine/threonine-rich domain, which we predicted to be involved in regulated protein interactions (Doi et al., 2006), and exon 19, the domain originally found to be essential for CLOCK function (Antoch et al., 1997). In SIRT1, the N-terminal region (aa 1-231) is necessary and sufficient for eliciting efficient interaction with CLOCK (FIG. 5F). This information is of interest because the same SIRT1 domain is involved in the interaction with other regulatory proteins. Specifically, it has been recently found to mediate the interaction with the histone methyltransferase SUV39H1 (Vaquero et al., 2007), a regulatory event that results in increased levels of the H3K9me3 modification and thereby control of heterochromatin formation.

### BMAL1 Acetylation at Lys537 is Regulated by SIRT1

Recently we have reported that BMAL1 is rhythmically acetylated by CLOCK and that this event is essential for control of circadian function (Hirayama et al., 2007). We have generated an antibody that specifically recognizes acetylated BMAL1 at Lys537 (FIG. 10). Because of the interplay between CLOCK and SIRT1, we suspected that the deacetylase that could regulate the dynamic levels of BMAL1 acetylation could be SIRT1. To identify which class of HDAC is responsible for deacetylation of BMAL1, we treated cultured cells expressing Myc-CLOCK and Flag-Myc-BMAL1 with class I and II inhibitor, trichostatin A (TSA), and/or class III inhibitor, NAM, for 6 hr and 16 hr, respectively. Acetylation of BMAL1 at Lys537 is significantly increased by NAM treatment but not by TSA treatment (FIG. 6A). We then coexpressed BMAL1 with CLOCK and confirmed that the anti-AcBMAL1 antibody readily recognizes CLOCK-induced acetylation at Lys537 (FIG. 6B). In the same assay, coexpression of SIRT1, but not SIRT2 and SIRT3, induced specific deacetylation of BMAL1 (FIG. 6B), indicating that SIRT1 specifically deacetylates BMAL1. We also confirmed that SIRT1 readily deacetylates BMAL1 in an in vitro deacetylation assay (FIG. 11). Importantly, the SIRT1(H363Y) enzymatically deficient mutant did not affect the acetylation state of BMAL1 (FIG. 6C). Furthermore, BMAL1 deacetylation by SIRT1 is responsive to NAD⁺ and significantly attenuated by NAM (FIG. 6D), suggesting that the acetylation of BMAL1 is an event regulated by cellular metabolism.

### SIRT1 Contributes to Circadian Control In Vivo

To determine the effect of SIRT1 on the cyclic acetylation of BMAL1 we first compared MEFs from WT mice and Sirt1−/− animals. Upon serum shock, acetylation at Lys537 is cyclic in WT cells, whereas it is sustained and mostly constant in Sirt1−/− MEFs (FIG. 7A). Interestingly, lack of acetylation appears to also influence BMAL1 phosphorylation levels (FIG. 7A). As phosphorylation has been linked to BMAL1 stability (Cardone et al., 2005; Kondratov et al., 2003), it is interesting to observe that BMAL1 appears indeed to be expressed at higher levels in the absence of SIRT1. These results suggest that SIRT1—controlled acetylation could constitute a critical regulatory step in the control of BMAL1 protein stability.

Next we sought to demonstrate the role of SIRT1 in vivo. To do so, we used tissue-specific Sirt1−/− mice in which the loxed gene was selectively deleted by albumin promoter-driven Cre recombinase in the liver. The original mutant mice have the unique deletion of exon 4 of the Sirt1 gene, which encodes the conserved SIRT1 catalytic domain (Cheng et al., 2003). Livers were collected from mice entrained at different times of the circadian cycle and used to analyze BMAL1 acetylation and gene expression levels. Paralleling the results obtained with the Sirt1−/− MEFs (FIG. 7A), BMAL1 acetylation is significantly increased and only mildly rhythmic in the livers from the mutant mice (FIG. 7B). BMAL1 phosphorylation is also slightly increased with respect to WT mice, although not to the extent observed in the Sirt1−/− MEFs (see also FIG. 12). Finally, expression of the circadian genes Cry 1 and Per2 is also significantly altered (FIG. 7C), reminiscent of the results obtained with the Sirt1−/− MEFs (FIG. 2).

### Discussion

A large array of metabolic processes follows the rhythmicity of the circadian cycle. The presence of molecular links that reveal functional wiring between the clock machinery and metabolic pathways has been invoked (Rutter et al., 2002; Schibler and Sassone-Corsi, 2002), and much compelling evidence has accumulated (Turek et al., 2005; Wijnen and Young, 2006). We have proposed that the HAT function of CLOCK may be controlled by changing cell energy levels or, conversely, could regulate them (Doi et al., 2006; Grimaldi et al., 2007). The finding that a core element of the clock machinery directly elicits histone modifications underscored the link between circadian physiology and chromatin remodeling. These notions suggested that NAD(H)-dependent energy pathways in the cell could influence the HAT function of CLOCK:BMAL1. We reasoned that CLOCK-mediated acetylation, and thereby transcriptional activation, could be counterbalanced by transcriptional silencing induced by NAD⁺-dependent HDACs (Imai et al., 2000; Landry et al., 2000). Intriguingly Sir2, a NAD⁺-dependent HDAC, had been functionally linked to Sas2 (Kimura et al., 2002; Suka et al., 2002), a protein of the MYST family of HATs to which CLOCK belongs (Doi et al., 2006; Nakahata et al., 2007).

Our results indicate that SIRT1 could function as a molecular rheostat of CLOCK-mediated HAT function, by modulating the timing of histone lysine acetylation (FIG. 7D). SIRT1 also modulates the circadian machinery by controlling the acetylation levels of BMAL1 (FIG. 6), a core circadian element whose CLOCK-induced acetylation is important for circadian physiology (Hirayama et al., 2007). BMAL1 is acetylated at a key, conserved lysine at position Lys537. We have shown that Lys537 acetylation increases the efficacy of the repressor CRY to silence CLOCK: BMAL1-mediated transcription, an event essential to obtaining proper circadian oscillations (Hirayama et al., 2007). Importantly, the oscillatory acetylation patterns of H3 and BMAL1 differ in their timing: BMAL1 acetylation is sustained at a circadian time when H3 acetylation is at minimal levels (at 24 hr post-serum shock; compare FIGS. 4A and 7A). This difference nicely fits the scenario of a dual role for CLOCK-mediated acetylation, implicated both in transcriptional activation of circadian promoters (acetylation of H3; Doi et al., 2006) and in their subsequent downregulation following acetylation of BMAL1- and CRY-mediated repression (Hirayama et al., 2007). These findings raise the fascinating possibility that CLOCK and SIRT1 enzymatic activities may be temporally regulated by additional post-translational modifications. Interestingly, H3 Lys14 acetylation was shown to be significantly modulated by the phosphorylation of the nearby Ser10 residue (Cheung et al., 2000b; Lo et al., 2000). Of relevance to circadian control, phosphorylation at Ser10 has been linked to light-induced activation of clock gene expression in the suprachiasmatic nucleus (SCN) (Crosio et al., 2000).

Another important question relates to whether SIRT1 would operate on other nonhistone circadian targets. CLOCK was found to interact with some nuclear receptors, including RARα and RXRα (McNamara et al., 2001). Since periodic availability of nuclear hormones has been implicated in the resetting of peripheral clocks (McNamara et al., 2001; Yin et al., 2007), and since SIRT1 has been found to control a number of nuclear receptors (see for example Li et al., 2007b), it is reasonable to speculate that the CLOCK-SIRT1 interaction described in this study represents a key event in the processes of fat and energy metabolism. In this respect, it is worth noting that PGC-1, a transcriptional coactivator that regulates energy metabolism and that acts in combination with SIRT1 (Nemoto et al., 2005; Rodgers et al., 2005), is rhythmically expressed in the liver and skeletal muscle and is required for cell-autonomous clock function (Liu et al., 2007; Sonoda et al., 2007). Thus, the CLOCK-SIRT1 interplay seems to occupy a privileged position in the control of gene expression by metabolites.

CLOCK and SIRT1 appear to be associated at all times of the circadian cycle (FIG. 5), suggesting that they would not only coordinately contribute to the dynamic oscillation of histone acetylation but also regulate a number of nonhistonic targets. The identification of additional molecular elements within the CLOCK:BMAL1/SIRT1 complex will define its functional features, leading to the unraveling of intracellular regulatory pathways yet poorly understood. In this respect, a fascinating connection is apparent between circadian metabolism, aging, and cancer. DNA damage accumulates with age and defects in DNA repair may lead to phenotypes reminiscent of premature aging (Lombard et al., 2005; Saunders and Verdin, 2007). The conceptual and functional link existing between the circadian clock and the cell cycle (Hunt and Sassone-Corsi, 2007; Chen and McKnight, 2007) has been extended to implicate the circadian machinery in the DNA-damage response (Collis and Boulton, 2007). The circadian genes per1 and tim have been shown to play an important role in DNA-damage control (Gery et al., 2006; Unsal-Kaçmaz et al., 2005), and phase resetting of the mammalian circadian clock is readily obtained by DNA-damaging agents (Oklejewicz et al., 2008). Finally, the role of SIRT1 in the aging process (Oberdoerffer and Sinclair, 2007; Bishop and Guarente, 2007) is intriguingly paralleled by recent observations of early aging and age-related pathologies observed in BMAL1-deficient mice (Kondratov et al., 2006). The far-reaching implications of our findings are thereby multiple, including the identification of novel strategies for the study of diabetes, obesity, and aging.

### References for Example 1

Andrews, N. C., and Faller, D. V. (1991). A rapid micropreparation technique for extraction of DNA-binding proteins from limiting numbers of mammalian cells. Nucleic Acids Res. 19, 2499.
Antoch, M. P., Song, E. J., Chang, A. M., Vitaterna, M. H., Zhao, Y., Wilsbacher, L. D., Sangoram, A. M., King, D. P., Pinto, L. H., and Takahashi, J. S. (1997). Functional identification of the mouse circadian Clock gene by transgenic BAC rescue. Cell 89, 655-667.
Bishop, N. A., and Guarente, L. (2007). Genetic links between diet and lifespan: shared mechanisms from yeast to humans. Nat. Rev. Genet. 8, 835-844. Bordone, L., and Guarente, L. (2005). Calorie restriction, SIRT1 and metabolism: understanding longevity. Nat. Rev. Mol. Cell. Biol. 6, 298-305.
Brunet, A., Sweeney, L. B., Sturgill, J. F., Chua, K. F., Greer, P. L., Lin, Y., Tran, H., Ross, S. E., Mostoslaysky, R., Cohen, H. Y., et al. (2004). Stress-dependent regulation of FOXO transcription factors by the SIRT1 deacetylase. Science 303, 2011-2015.
Cardone, L., Hirayama, J., Giordano, F., Tamaru, T., Palvimo, J. J., and Sassone-Corsi, P. (2005). Circadian clock control by SUMOylation of BMAL1. Science 309, 1390-1394. Chen, Z., and McKnight, S. L. (2007). A conserved DNA damage response pathway responsible for coupling the cell division cell cycle to the circadian and metabolic cycles. Cell Cycle 6, 2906-2912.
Cheng, H. L., Mostoslaysky, R., Saito, S., Manis, J. P., Gu, Y., Patel, P., Bronson, R., Appella, E., Alt, F. W., and Chua, K. F. (2003). Developmental defects and p53 hyperacetylation in Sir2 homolog (SIRT1)-deficient mice. Proc. Natl. Acad. Sci. USA 100, 10794-10799.
Cheung, P., Allis, C. D., and Sassone-Corsi, P. (2000a). Signaling to chromatin through histone modifications. Cell 103, 263-271.
Cheung, P., Tanner, K. G., Cheung, W. L., Sassone-Corsi, P., Denu, J. M., and Allis, C. D. (2000b). Synergistic coupling of histone H3 phosphorylation and acetylation in response to epidermal growth factor stimulation. Mol. Cell. 5, 905-915.
Chopra, V. S., and Mishra, R. K. (2005). To SIR with Polycomb: linking silencing mechanisms. Bioessays 27, 119-121.
Collis, S. J., and Boulton, S. J. (2007). Emerging links between the biological clock and the DNA damage response. Chromosoma 116, 331-339.
Crosio, C., Cermakian, N., Allis, C. D., and Sassone-Corsi, P. (2000). Light induces chromatin modification in cells of the mammalian circadian clock. Nat. Neurosci. 3, 1241-1247.
Doi, M., Hirayama, J., and Sassone-Corsi, P. (2006). Circadian regulator CLOCK is a histone acetyltransferase. Cell 125, 497-508.
Gery, S., Komatsu, N., Baldjyan, L., Yu, A., Koo, D., and Koeffler, H. P. (2006). The circadian gene per1 plays an important role in cell growth and DNA damage control in human cancer cells. Mol. Cell. 22, 375-382.
Grimaldi, B., Nakahata, Y., Sahar, S., Kaluzova, M., Gauthier, D., Pham, K., Patel, N., Hirayama, J., and Sassone-Corsi, P. (2007). Chromatin remodeling and circadian control: Master regulator CLOCK is an enzyme. Cold Spring Harb. Symp. Quant. Biol. 72, 105-112.
Grunstein, M. (1997). Histone acetylation in chromatin structure and transcription. Nature 389, 349-352.
Hardin, P. E., and Yu, W. (2006). Circadian transcription: passing the HAT to CLOCK. Cell 125, 424-426.
Hirayama, J., Sahar, S., Grimaldi, B., Tamaru, T., Takamatsu, K., Nakahata, Y., and Sassone-Corsi, P. (2007). CLOCK-mediated acetylation of BMAL1 controls circadian function. Nature 450, 1086-1090.
Hunt, T., and Sassone-Corsi, P. (2007). Riding tandem: circadian clocks and the cell cycle. Cell 129, 461-464.
Imai, S., Armstrong, C. M., Kaeberlein, M., and Guarente, L. (2000). Transcriptional silencing and longevity protein Sir2 is an NAD-dependent histone deacetylase. Nature 403, 795-800.
Kimura, A., Umehara, T., and Horikoshi, M. (2002). Chromosomal gradient of histone acetylation established by Sas2p and Sir2p functions as a shield against gene silencing. Nat. Genet. 32, 370-377.
Kondratov, R. V., Chemov, M. V., Kondratova, A. A., Gorbacheva, V. Y., Gudkov, A. V., and Antoch, M. P. (2003). BMAL1-dependent circadian oscillation of nuclear CLOCK: posttranslational events induced by dimerization of transcriptional activators of the mammalian clock system. Genes Dev. 17, 1921-1932.
Kondratov, R. V., Kondratova, A. A., Gorbatcheva, V. Y., Vykhovanets, O. V., and Antoch, M. P. (2006). Early aging and age-related patologies in mice deficient in BMAL1, the core component of the circadian clock. Genes Dev. 20, 1868-1873.
Kouzarides, T. (2007). Chromatin modifications and their function. Cell 128, 693-705.
Kovacs, J. J., Murphy, P. J., Gaillard, S., Zhao, X., Wu, J. T., Nicchitta, C. V., Yoshida, M., Toft, D. O., Pratt, W. B., and Yao, T. P. (2005). HDAC6 regulates Hsp90 acetylation and chaperone-dependent activation of glucocorticoid receptor. Mol. Cell. 18, 601-607.
Kurdistani, S. K., and Grunstein, M. (2003). Histone acetylation and deacetylation in yeast. Nat. Rev. Mol. Cell. Biol. 4, 276-284.
Ladurner, A. G. (2006). Rheostat control of gene expression by metabolites. Mol. Cell. 24, 1-11.
Lagouge, M., Argmann, C., Gerhart-Hines, Z., Meziane, H., Lerin, C., Daussin, F., Messadeq, N., Milne, J., Lambert, P., Elliott, P., et al. (2006). Resveratrol improves mitochondrial function and protects against metabolic disease by activating SIRT1 and PGC-1alpha. Cell 127, 1109-1122.
Landry, J., Sutton, A., Tafrov, S. T., Heller, R. C., Stebbins, J., Pillus, L., and Sternglanz, R. (2000). The silencing protein SIR2 and its homologs are NADdependent protein deacetylases. Proc. Natl. Acad. Sci. USA 97, 5807-5811.
Lee, C., Etchegaray, J. P., Cagampang, F. R., Loudon, A. S., and Reppert, S. M. (2001). Posttranslational mechanisms regulate the mammalian circadian clock. Cell 107, 855-867.
Li, B., Carey, M., and Workman, J. L. (2007a). The role of chromatin during transcription. Cell 128, 707-719.
Li, X., Zhang, S., Blander, G., Tse, J. G., Krieger, M., and Guarente, L. (2007b). SIRT1 deacetylates and positively regulates the nuclear receptor LXR. Mol. Cell. 28, 91-106.
Liu, C., Li, S., Liu, T., Borjigin, J., and Lin, J. D. (2007). Transcriptional coactivator PGC-1alpha integrates the mammalian clock and energy metabolism. Nature 447, 477-481.
Lo, W. S., Trievel, R. C., Rojas, J. R., Duggan, L., Hsu, J. Y., Allis, C. D., Marmorstein, R., and Berger, S. L. (2000). Phosphorylation of serine 10 in histone H3 is functionally linked in vitro and in vivo to Gcn5-mediated acetylation at lysine 14. Mol. Cell. 5, 917-926.
Lombard, D. B., Chua, K. F., Mostoslaysky, R., Franco, S., Gostissa, M., and Alt, F. W. (2005). DNA repair, genome stability, and aging. Cell 120, 497-512.
Luo, J., Nikolaev, A. Y., Imai, S., Chen, D., Su, F., Shiloh, A., Guarente, L., and Gu, W. (2001). Negative control of p53 by Sir2alpha promotes cell survival under stress. Cell 107, 137-148.
Mal, A., Sturniolo, M., Schiltz, R. L., Ghosh, M. K., and Harter, M. L. (2001). A role for histone deacetylase HDAC1 in modulating the transcriptional activity of MyoD: inhibition of the myogenic program. EMBO J. 20, 1739-1753.
Martinez-Balbas, M. A., Bauer, U. M., Nielsen, S. J., Brehm, A., and Kouzarides, T. (2000). Regulation of E2F1 activity by acetylation. EMBO J. 19, 662-671.
McNamara, P., Seo, S. P., Rudic, R. D., Sehgal, A., Chakravarti, D., and FitzGerald, G. A. (2001). Regulation of CLOCK and MOP4 by nuclear hormone receptors in the vasculature: A humoral mechanism to reset a peripheral clock. Cell 105, 877-889.
Motta, M. C., Divecha, N., Lemieux, M., Kamel, C., Chen, D., Gu, W., Bultsma, Y., McBurney, M., and Guarente, L. (2004). Mammalian SIRT1 represses forkhead transcription factors. Cell 116, 551-563.
Nakahata, Y., Grimaldi, B., Sahar, S., Hirayama, J., and Sassone-Corsi, P. (2007). Signaling to the circadian clock: plasticity by chromatin remodeling. Curr. Opin. Cell Biol. 19, 230-237.
Nemoto, S., Fergusson, M. M., and Finkel, T. (2005). SIRT1 functionally interacts with the metabolic regulator and transcriptional coactivator PGC-1 {alpha}. J. Biol. Chem. 280, 16456-16460.
Nowak, D. E., Tian, B., and Brasier, A. R. (2005). Two-step cross-linking method for identification of NF-kappaB gene network by chromatin immunoprecipitation. Biotechniques 39, 715-725.
Oberdoerffer, P., and Sinclair, D. A. (2007). The role of nuclear architecture in genomic instability and ageing. Nat. Rev. Mol. Cell. Biol. 8, 692-702.
Oklejewicz, M., Destici, E., Tamanini, F., Hut, R. A., Janssens, R., and van der Horst, G. T. J. (2008). Phase resetting of themammalian circadian clock by DNA damage. Curr. Biol. 18, 286-291.
Panda, S., Antoch, M. P., Miller, B. H., Su, A. I., Schook, A. B., Straume, M., Schultz, P. G., Kay, S. A., Takahashi, J. S., and Hogenesch, J. B. (2002). Coordinated transcription of key pathways in the mouse by the circadian clock. Cell 109, 307-320.
Pando, M. P., Morse, D., Cermakian, N., and Sassone-Corsi, P. (2002). Phenotypic rescue of a peripheral clock genetic defect via SCN hierarchical dominance. Cell 110, 107-117.
Peterson, C. L., and Laniel, M. A. (2004). Histones and histone modifications. Curr. Biol. 14, R546-R551.
Picard, F., Kurtev, M., Chung, N., Topark-Ngarm, A., Senawong, T., Machado De Oliveira, R., Leid, M., McBurney, M. W., and Guarente, L. (2004). Sirt1 promotes fat mobilization in white adipocytes by repressing PPAR-gamma. Nature 429, 771-776.
Ripperger, J. A., and Schibler, U. (2006). Rhythmic CLOCK-BMAL1 binding to multiple E-box motifs drives circadian Dbp transcription and chromatin transitions. Nat. Genet. 38, 369-374.
Rodgers, J. T., Lerin, C., Haas, W., Gygi, S. P., Spiegelman, B. M., and Puigserver, P. (2005). Nutrient control of glucose homeostasis through a complex of PGC-1alpha and SIRT1. Nature 434, 113-118.
Rutter, J., Reick, M., and McKnight, S. L. (2002). Metabolism and the control of circadian rhythms. Annu. Rev. Biochem. 71, 307-331.
Saunders, L. R., and Verdin, E. (2007). Sirtuins: critical regulators at the crossroads between cancer and aging. Oncogene 26, 5489-5504.
Sauve, A. A., Wolberger, C., Schramm, V. L., and Boeke, J. D. (2006). The biochemistry of sirtuins. Annu. Rev. Biochem. 75, 435-465.
Schibler, U., and Sassone-Corsi, P. (2002). A web of circadian pacemakers. Cell 111, 919-922.
Sonoda, J., Mehl, I. R., Ching, L. W., Nofsinger, R. R., and Evans, R. M. (2007). PGC-1 beta controls mitochondrial metabolismto modulate circadian activity, adaptive thermogenesis, and hepatic steatosis. Proc. Natl. Acad. Sci. USA 104, 5223-5228.
Storch, K. F., Lipan, O., Leykin, I., Viswanathan, N., Davis, F. C., Wong, W. H., and Weitz, C. J. (2002). Extensive and divergent circadian gene expression in liver and heart. Nature 417, 78-83.
Strahl, B. D., and Allis, C. D. (2000). The language of covalent histone modifications. Nature 403, 41-45.
Struhl, K. (1998). Histone acetylation and transcriptional regulatory mechanisms. Genes Dev. 12, 599-606.
Suka, N., Luo, K., and Grunstein, M. (2002). Sir2p and Sas2p opposingly regulate acetylation of yeast histone H4 lysinel6 and spreading of heterochromatin. Nat. Genet. 32, 378-383.
Tissenbaum, H. A., and Guarente, L. (2001). Increased dosage of a sir-2 gene extends lifespan in Caenorhabditis elegans. Nature 410, 227-230.
Travnickova-Bendova, Z., Cermakian, N., Reppert, S. M., and Sassone-Corsi, P. (2002). Bimodal regulation of mPeriod promoters by CREB-dependent signaling and CLOCK/BMAL1 activity. Proc. Natl. Acad. Sci. USA 99, 7728-7733.
Turek, F. W., Joshu, C., Kohsaka, A., Lin, E., Ivanova, G., McDearmon, E., Laposky, A., Losee-Olson, S., Easton, A., Jensen, D. R., et al. (2005). Obesity and metabolic syndrome in circadian Clock mutant mice. Science 308, 1043-1045.
Ueda, H. R., Chen, W., Adachi, A., Wakamatsu, H., Hayashi, S., Takasugi, T., Nagano, M., Nakahama, K., Suzuki, Y., Sugano, S., et al. (2002). A transcription factor response element for gene expression during circadian night. Nature 418, 534-539.
Unsal-Kac, maz, K., Mullen, T. E., Kaufmann, W. K., and Sancar, A. (2005). Coupling of human circadian and cell cycles by the timeless protein. Mol. Cell. Biol. 25, 3109-3116.
Vaquero, A., Scher, M., Erdjument-Bromage, H., Tempst, P., Serrano, L., and Reinberg, D. (2007). SIRT1 regulates the histone methyl-transferase SUV39H1 during heterochromatin formation. Nature 450, 440-444.
Vaziri, H., Dessain, S. K., Ng Eaton, E., Imai, S. I., Frye, R. A., Pandita, T. K., Guarente, L., and Weinberg, R. A. (2001). hSIR2(SIRT1) functions as an NADdependent p53 deacetylase. Cell 107, 149-159.
Wade, P. A., and Wolffe, A. P. (1997). Histone acetyltransferases in control. Curr. Biol. 7, R82-R84.
Wijnen, H., and Young, M. W. (2006). Interplay of circadian clocks and metabolic rhythms. Annu. Rev. Genet. 40, 409-448.
Workman, J. L., and Kingston, R. E. (1998). Alteration of nucleosome structure as a mechanism of transcriptional regulation. Annu. Rev. Biochem. 67, 545-579.
Yamamoto, T., Nakahata, Y., Soma, H., Akashi, M., Mamine, T., and Takumi, T. (2004). Transcriptional oscillation of canonical clock genes in mouse peripheral tissues. BMC Mol. Biol. 5, 18.
Yang, X. J., and Seto, E. (2008). The Rpd3/Hda1 family of lysine deacetylases: from bacteria and yeast to mice and men. Nat. Rev. Mol. Cell. Biol. 9, 206-218.
Yin, L., Wu, N., Curtin, J. C., Qatanani, M., Szwergold, N. R., Reid, R. A., Waitt, G. M., Parks, D. J., Pearce, K. H., Wisely, G. B., and Lazar, M. A. (2007). Rev-erbalpha, a heme sensor that coordinates metabolic and circadian pathways. Science 318, 1786-1789.

### Example 2

### Circadian Control of the NAD⁺ Salvage Pathway by CLOCK-SIRT1

### Materials and Methods

Animals Male BALB/c mice and clock/clock mutant mice were housed under 12-h light/12-h dark (LD) cycles over 2 weeks. All protocols using animals in this study were approved and reviewed by the Institutional Animal Care and Use Committee of the University of California, Irvine.

Plasmids FLAG-tagged mClock/pSG5 and Myc-tagged mBmall/pCS2 have been described (6, 7). Human Nampt promoter, referred as “−1637/pGL4.10” in this study, was kind gift of Dr. Kazuya Yamagata. -1637/pGL4.10 was digested by Acc65I and AatII, Tthl11I, or SmaI followed by blunting by Klenow enzyme to construct -1081/pGL4.10, -734/pGL4.10, or -170/pGL4.10, respectively. pRL-CMV vector was purchased from Promega.

Antibodies and reagents Antibodies against acetyl-Histone H3 (catalog no. 06-599) and SIRT1 (catalog no. 07-131) were purchased from Millipore and antibody against Clock (catalog no. sc-6927) and normal rabbit IgG (catalog no. sc-2027) were purchased from Santa Cruz Biotechnology. Antibody against Nampt (OMNI379) was purchased from ALEXIS Biochemicals. Antibody against BMAL1 and acetyl-lysine 537 BMAL1 were described (51; see Example 1 above, also see 8). FK866 was purchased from Axon Medchem. NAD (N8535), nicotinamide (N0636), and 2-chloroadenosine (C5134) were purchased from SIGMA.

Cell culture, Transient transfections and Luciferase assays All cells used in this study were grown at 37° C. and 5% CO₂. Wild type, clock/clock mutant and cry ½ double deficient MEF cells were grown in Dulbecco's Modified Eagle Medium (4.5 g/L glucose) supplemented with 10% fetal bovine serum (FBS) and antibiotics. MEF cells established from homozygous Sirt1−/− mice and counterpart wild type MEF cells were cultured in Dulbecco's Modified Eagle Medium (4.5 g/L glucose) supplemented with 7.5% newborn bovine serum, 2.5% FBS and antibiotics. JEG3 cells were grown in Basal Medium Eagle supplemented with 10% FBS and antibiotics. For transient transfection luciferase assays, JEG3 were transfected with various combinations of expression and reporter plasmids using FuGENE HD (Roche Molecular Biochemicals), according to the manufacturer's protocol. Luciferase activities were assayed with the dual-luciferase reporter assay system (Promega) in a Berthold luminometer.

NAMPT inhibitor “FK866” treatment experiments MEFs in growing phase (−50% confluent) were pre-treated with 10 nM FK866 dissolved in ethanol/serum-free DMEM 16 hr prior to high serum treatment. After 2 hr serum treatment, medium were changed to serum-free medium containing 10 nM FK866.

Preparation of Cell Extracts from Cultured Cell Lines and Mice Livers for preparation of total cell extracts, MEFs were washed twice with phosphate buffered saline (PBS) and lysed in RIPA buffer (50 mM Tris pH 8.0, 150 mM NaCl, 5 mM EDTA, 15 mM MgCl2, 1% NP40, 1× protease inhibitor cocktail (Roche), 1 mM DTT, 1 μM trichostatin A (TSA), 10 mM nicotinamide (NAM), 10 mM NaF, 1 mM PMSF). For preparation of mice liver total extracts, liver samples were homogenized in RIPA buffer by dounce homogenizer.

Chromatin Immunoprecipitation (ChIP) Assays and Quantitative Real-time Reverse Transcription (RT)-PCR We performed the conventional and dual cross-linking ChIP assays as described (8). Each quantitative real-time RT-PCR was performed using the Chromo4 real time detection system (BIO-RAD). The PCR primers for mNampt mRNA, mNmnat1˜3 mRNAs, mNampt TSS, and mNampt 3′R were designed with a real-time PCR primer design tool, Real-Time PCR Primer Design (available at https://www.genscript.com/ssl-bin/app/primer), and the sequences of the primers were as follows: mNampt mRNA FW, GGT CAT CTC CCG ATT GAA GT (SEQ ID NO: 1); mNampt mRNA RV, TCA ATC CAA TTG GTA AGC CA (SEQ ID NO: 2); mNmnat1 mRNA FW, TGG AGA CTG TGA AGG TGC TC (SEQ ID NO: 3); mNmnat1 mRNA RV, TGA GCT TTG TGG GTA ACT GC (SEQ ID NO: 4); mNmnat2 mRNA FW, AGA ATT CCG ACT GGA TCA GG (SEQ ID NO: 5); mNmnat2 mRNA R^{V}, GGT CAC CCT CTT CAT CAG GT (SEQ ID NO: 6); mNmnat3 mRNA FW, CCG TCA TCA CCT ACA TCA GG (SEQ ID NO: 7); mNmnat3 mRNA RV, AGC CAG TCT TTC CTT TCC CT (SEQ ID NO: 8); mNampt TSS FW, GTG ACG GTC GGC TTT AGG (SEQ ID NO: 9); mNampt TSS RV, GGA CTG AGG AGG ACG TGA G (SEQ ID NO: 10); mNampt 3′R FW, CTT SCG CGA ATG TTT AGG CA (SEQ ID NO: 11); mNampt 3′R RV, GCA TAT TAG AGC CAC AGG CA (SEQ ID NO: 12). For a 20 μl PCR, 50 ng of cDNA template was mixed with the primers (final concentrations of 200 nM), and 10 μl of iQ™ SYBR Green Supermix (BIO-RAD). The reaction was first incubated at 95° C. for 3 min, followed by 40 cycles at 95° C. for 30 s and 60° C. for 1 min.

NAD⁺ and nicotinamide measurements by LC/MSⁿ analyses 50% serum treated-MEFs were washed and harvested with ice-cold PBS. Cells were centrifuged (3,000 rpm, 5 min at 4° C.) and kept at −80° C. until sample preparation. Cells were resuspended with 200 μl of extraction buffer (99% 5 mM ammonium formate/1% methanol containing 1 μM 2-chloroadenosine as an internal standard) and sonicated. After centrifugation(15,000 rpm, 10 min at 4° C.), 20 μl of resultant supernatant was kept for protein measurement, the rest of supernatant was filtered with a 0.22 μM filter followed by a regenerated cellulose 3000 molecular weight cut-off Microcon YM-3 filter (Millipore) to remove the cellular debris and proteins. We identified and quantified NAD⁺ and nicotinamide (NAM) by LC/MS″, using a 1100-LC system (Agilent Technologies, Palo Alto, Calif.) equipped with a Ion Trap XCT (Agilent Technologies). Analytes were separated using a ZORBAX SB-CN column (2.1×150 mm i.d., 5 μm, Agilent Technologies, Wilmington, Del.) maintained at 30° C. Mobile phase was water containing 5 mM ammonium acetate and 0.25% acetic acid (A) and methanol containing 5 mM ammonium acetate and 0.25% acetic acid (B). A gradient (100% to 50% of A in 10 min and from 50% to 30% of A in 15 min) was applied at a flow rate of 0.15 ml/min. Total run time was 19 min and post-time was 15 min (100% of A). Injection volume was 10 μl. Detection was set in the positive mode, capillary voltage was 4.0 kV, skim1 40V, and capillary exit 140V. N₂ was used as drying gas at a flow rate of 10 liters/min, temperature of 350° C. and nebulizer pressure of 60 PSI. Helium was used as collision gas. Cell-derived NAD⁺ and NAM were identified by comparison of their LC retention times and MS″ fragmentation patterns with those of authentic standards (Sigma-Aldrich, St Louis, Mo.). We acquired full-scan MS² spectra of NAD⁺ and 2-chloroadenosine by multiple reaction monitoring with isolation width of 2 and fragmentation voltage of 1.1 V. Ion charge control was on, smart target set at 100,000 and max accumulation time at 200 ms at 26,000 m/z per sec. Extracted ion chromatograms were used to quantify NAM (m/z 123.3) and NAD⁺ (m/z 664.3>523.5) using 2-chloroadenosine (m/z 302.3>170.5). Limits of quantification were 1 pmol for both NAM and NAD⁺. Detection and analysis were controlled by Agilent/Bruker Daltonics software version 5.2.

In silico circadian elements search The sequences were downloaded from the NCBI Gene database. Each Nampt gene sequence spanning from 2 kb upstream of the translation start codon was examined. Multiple sequence alignments of these sequences were obtained by ClustalW 2.0.5 with default parameters. The binding elements were then searched from these alignments using a pattern finding tool, fuzznuc, with the following consensus sequences allowing for a 2-base mismatch:

| | |
|---|---|
| RORE: [AT]A[AT]NT[AG]GGTCA | (SEQ ID NO: 13) |
| DBPE: [GA]T[GT]A[TC]GTAA[TC] | (SEQ ID NO: 14) |
| E-Box: CACGTG. | (SEQ ID NO: 15) |

### Introduction

A remarkable variety of fundamental physiological functions in most organisms is controlled by the circadian clock. This is a time-tracking system intrinsic to most organisms that enables the adaptation to environmental changes. Disruption of circadian rhythms has profound influence to human health and has been linked to depression, insomnia, jet lag, coronary heart disease and a variety of neurodegenerative diseases. Thereby, the molecular mechanisms governing the circadian clock constitute a very attractive hold for the understanding of the links to physiology and metabolism, representing potential tools for the development of therapeutic strategies.

Remarkably, 10-15% of all mammalian transcripts undergo circadian fluctuations in their expression levels. Thus, genome-wide mechanisms must operate in order to insure such global transcriptional regulation. Our studies described herein (see also Cell 125: 497-508, 2006) have established that CLOCK, a master controller of circadian rhythms, directly modifies chromatin. CLOCK possesses intrinsic enzymatic histone acetyltransferase (HAT) activity, demonstrating that control of chromatin remodeling constitutes a key regulatory step governing the circadian clock machinery.

A central question has been how CLOCK-mediated HAT function is counterbalanced. The question has physiological relevance since we found that SIRT1 is the HDAC that exerts this function (8). Importantly, acetylation could be linked to changes in cellular metabolism, resulting in modulated chromatin remodeling. Important implications for metabolism and neuronal function of this type of regulation exist, and we have reviewed them recently (52, 53). We also demonstrated that the metabolite NAD+, the coenzyme for SIRT1, oscillates in a circadian manner. This observation established a tight link between circadian biology and cellular metabolism, indicating that regulation goes both ways (53).

Cells sense and adapt to the environment using an array of sophisticated signaling pathways that provide plasticity to all physiological responses. The circadian system, whose functionality and molecular organization share remarkable similarities among species (1), is central to shape the capacity of most organisms to adapt and anticipate to changing conditions. These notions underscore the intimate interplay between circadian clocks and metabolic rhythms (2, 3). Indeed, a remarkable array of metabolic and physiological processes display daily oscillations (4, 5), leading to the question of what molecular gears are utilized by the circadian system to ‘sense’ metabolism, and whether the clock itself could influence metabolic responses. In this respect, a notable finding from a molecular standpoint relates to one of the core clock elements: the master regulator CLOCK has an intrinsic acetyltransferase enzymatic activity, which enables circadian chromatin remodeling by histone acetylation (6) and modification of other non-histone proteins, including its own partner and circadian regulator BMAL1 (7). A direct link with cellular metabolism was recently discovered. The histone deacetylase (HDAC) that counterbalances the HAT function of CLOCK is SIRT1 (8, 9), a protein of the sirtuin class whose enzymatic activity is dependent on intracellular NAD⁺ levels (10-12). The participation of SIRT1 in circadian control uncovered a unique example of control of gene expression by metabolites (8, 9), and provided a molecular interpretation of the fundamental role that circadian rhythms have in the physiological response of all metabolic tissues, including liver, muscle and fat (2).

The enzymatic activity of SIRT1 is under the control of certain metabolic cofactors and inhibitors. While NAD⁺ is SIRT1's natural co-substrate, the reduced form NADH and the by-product of NAD⁺ consumption, nicotinamide (NAM), repress the activity of SIRT1 (8, 13-15), generating an enzymatic feedback loop on the HDAC function of this enzyme. Indeed, it is established that fluctuations in NAD⁺ or changes in the NAD⁺/NADH ratio and NAM concentrations directly influence SIRT1 function (16). Two main systems determine NAD⁺ levels in the cell, the de novo biosynthesis from tryptophan and the NAD⁺ salvage pathway (17). A critical step of this latter pathway is controlled by the enzyme nicotinamide phosphoribosyl-transferase (Nampt), also known as visfatin or PBEF (18) that catalyzes the first step in the biosynthesis of NAD⁺ from NAM.

Regulation of NAD⁺ salvage pathway

Accumulating evidence illustrates the critical role played by Nampt in cellular metabolism. The yeast functional equivalent of Nampt, the nicotinamidase PNC1, is necessary and sufficient for life span extension induced by calorie restriction and low-intensity stress in a Sir2-dependent manner (15). Modulation of the nuclear NAD⁺ salvage pathway delays aging even under conditions of unaltered steady-state NAD⁺ levels (19). In mammalian cells, Nampt slows down senescence of human cells (20) and promotes survival during genotoxic stress (21, 22). Importantly, the Nampt gene expression is dynamic since being inducible by various agents (22-24) and specifically being stress- and nutrient-responsive (22), indicating that its control is central in governing the intracellular NAD⁺: NAM balance.

Unfortunately, while at least some aspects of Nampt gene expression and intracellular NAD⁺:NAM balance are known in the art, it still remains unclear how regulation of Nampt gene expression is controlled in a cell.

It has now been discovered that circadian regulation can be achieved by oscillating levels of intracellular NAD⁺. More particularly, the circadian core regulators CLOCK:BMAL1 modulate the circadian expression of the Nampt gene, which in turn controls the intracellular levels of NAD⁺, a key metabolite that functions as coenzyme of SIRT1. The NAD⁺ levels oscillate in a circadian manner, thereby inducing the HDAC activity of SIRT1 in a rhythmic fashion. CLOCK:BMAL1 and SIRT1 are in the same transcription/chromatin remodeling complex, and together control target promoters, specifically inducing the oscillatory expression of the Nampt gene. Thus, the findings reported herein reveal interlocking of two auto-regulatory systems, in which a classical transcription circadian loop is coupled to an enzymatic feedback loop.

A remarkable array of metabolic and physiological processes display circadian oscillations. Previous studies have shown that the core circadian regulator, CLOCK, is a histone acetyltransferase whose activity is counterbalanced by the NAD⁺-dependent histone deacetylase SIRT1.

Since variations in the NAD⁺ and NAM levels are thought to be critical for sirtuins activity, and based on our recent finding of the involvement of SIRT1 in circadian control, we reasoned that circadian regulation may be achieved by oscillating levels of intracellular NAD⁺. Indeed, while SIRT1 and CLOCK proteins levels do not seem to oscillate, the acetylation of targets of CLOCK-mediated HAT function, such as K14 of histone H3 and K537 of BMAL1 (6, 7) is circadian.

As described in more detail below, it has been found that intracellular NAD⁺ levels cycle with a 24 h rhythm, an oscillation driven by the circadian clock. CLOCK:BMAL1 regulate the circadian expression of Nampt (nicotinamide phosphoribosyltransferase), a rate limiting step enzyme in the NAD⁺ salvage pathway. SIRT1 is recruited to the Nampt promoter and contributes to the circadian synthesis of its own coenzyme. Using the specific inhibitor FK866, it was found that Nampt is required to modulate circadian gene expression as well as BMAL1 circadian acetylation. Based on these findings, it is particularly contemplated that an interlocked transcriptional-enzymatic feedback loop governs the molecular interplay between cellular metabolism and circadian rhythms.

More specifically, and based on the above referenced observations related to Nampt gene expression and NAD⁺ levels, NAD⁺ levels were measured along the circadian cycle. To do so wild type mouse embryo fibroblasts (MEFs) were serum-entrained, an approach that faithfully recapitulates the physiological regulation of the circadian clock (25). Cellular NAD⁺ levels were measured by liquid chromatography coupled to tandem mass spectrometry (LC/MS″) from total extracts prepared at various time intervals after the serum shock. Importantly, cellular NAD⁺ showed circadian oscillation with a remarkable variation in amplitude reproducible over a large number of experiments of about 2.5 fold (FIG. 13A). The average NAD⁺ concentration of 25 pmol/μg protein (approximately 60 μM) found in MEFs is in keeping with recent reports for rat axons using LC coupled to ultraviolet detector (9 pmol/μg protein) (26), mouse erythrocyte using LC/MS/MS (368 μM) (27) and HEK293 cells using LC/MALDI/MS (365 μM) (22).

To establish whether NAD⁺ oscillation is driven by the circadian clock, cellular NAD⁺ levels were next analyzed in MEFs originated from the clock/clock mutant mice (c/c), which are totally arrhythmic because the molecular clock mechanism is disrupted (28). Importantly, NAD⁺ levels in entrained c/c mutant MEFs do not oscillate (FIG. 13B and FIG. 17), a result confirmed in MEFs from the arrhythmic Cry1/Cry2 double mutant mice (not shown). The total cellular NAM levels measured by LC/MSⁿ in entrained wt MEFs also showed oscillation, albeit more moderate, with a phase opposite to the one of NAD⁺. Also NAM oscillation was abolished in c/c MEFs (FIG. 13D). Thus, the circadian clock controls the levels of these metabolites. This analysis provided also another important clue: NAD⁺ and NAM levels are significantly lower in c/c MEFs as compared to wt cells (for NAD⁺ only 1.1 pmol/μg protein, about 5% of that in wt MEFs; FIGS. 13C and E). This notion points to an involvement of the NAD⁺ salvage pathway and, because of its dynamic regulation, specifically to Nampt whose critical function in NAD⁺ production has been shown to be predominant in several cell types, including NIH 3T3, HepG2, SHSY5Y and OC-NYH (21, 29-31). Furthermore, increased flux through the NAD⁺ salvage pathway is responsible for sirtuin-dependent responses even under conditions of unaltered steady-state NAD⁺ levels (19).

### Circadian Control of Nampt

The finding that NAD⁺ levels oscillate in a circadian clock-dependent manner (FIG. 13) prompted analysis the expression of metabolic enzymes operating within the NAD⁺ salvage pathway in MEFs and mouse liver.

The three isoforms of nicotinamide mononucleotide adenylyltransferase, NMNAT1, NMNAT2 and NMNAT3, are central in NAD biosynthesis, catalyzing the condensation of nicotinamide mononucleotide (NMN) or nicotinic acid mononucleotide (NaMN) with the AMP moiety of ATP to form NAD⁺ (FIG. 14A). The expression of these three enzymes is mostly constant, or marginally oscillatory, along the circadian cycle in the liver (FIG. 18). Attention was focused on Nampt, which has been shown to operate as the rate limiting enzyme in the production of NAD⁺ within the NAD⁺ salvage pathway (18, 21, 22, 32). Differently from the NMNATs, the expression profile of Nampt is drastically circadian in livers from mice entrained at different times of the circadian cycle. Importantly, Nampt circadian oscillation is virtually absent in livers from c/c mice (FIG. 14B). Rhythmic expression is observed also in serum-entrained wt MEFs (FIG. 14C), where the circadian profile of Nampt parallels the one of Dbp, (FIG. 14C) and Per2 (not shown). Again, Nampt oscillation is abolished in MEFs from c/c mutant mice (FIG. 14C), demonstrating that it is under the control of the circadian clock.

Although Nampt rhythmic expression has been reported in microarray profiling studies (33-36) the molecular pathways of its control have not been elucidated. To uncover the molecular mechanism which confers circadian expression to the Nampt gene the inventor embarked in a promoter analysis (FIG. 15A). A search for circadian clock elements by in silico analyses revealed the presence of three putative E-boxes highly conserved in the human, rat, and mouse Nampt genes (FIG. 15B) and partially conserved in the corresponding zebrafish and chicken genes (not shown). Other circadian promoter elements (37, 38), such as the ROR binding element (RORE) and the DBP binding element (DBPE), are not present within 2 kb upstream from the transcription start site of human, rat, and mouse Nampt genes. To address whether the three E-boxes present in the promoter are functional the inventor transiently co-expressed a luciferase-based Nampt reporter with vectors encoding the CLOCK and BMAL1 core clock proteins in cultured JEG3 human cells. The Nampt promoter is readily activated by CLOCK:BMAL1 (FIG. 15C). While most of the promoter could be deleted with no major effect on its activity, deletion of the three E-boxes made it unresponsive to CLOCK:BMAL1, underscoring that Nampt is a clock-controlled gene.

Next, chromatin immunoprecipitation (ChIP) was performed to establish whether CLOCK:BMAL1 physically associate to the E-boxes on the Nampt promoter. Using MEF nuclear extracts and dual cross-linking ChIP assays (8, 39) it was revealed that CLOCK:BMAL1 bind to the promoter region spanning the E-boxes, but not to the Nampt 3′ UTR (FIG. 15D, E). Importantly, CLOCK and BMAL1 bind to the Nampt E-boxes in a time-dependent manner (FIG. 15D, E), consistent with Nampt mRNA circadian expression (see FIG. 14B, C). The inventor has recently demonstrated that SIRT1 is in a complex with CLOCK:BMAL1, contributing to the circadian control of K9/14-H3 and BMAL1 acetylation (see Example 1 above, also see ref. 8). Thus, it was examined whether SIRT1 would be recruited to the Nampt promoter. Dual cross-linking ChIP assays over the E-boxes region showed that SIRT1 binds to the Nampt E-boxes in a time-dependent manner, following the circadian timing of CLOCK:BMAL1 recruitment (FIGS. 15D and E). These results indicate that, as previously shown for Dbp and Per2 (see Example 1 above, also see ref. 8), CLOCK and SIRT1 contribute to the circadian chromatin remodeling at the promoter. As NAD⁺ intracellular levels directly influence the HDAC activity of SIRT1 (10-12), these findings reveal the presence of an enzymatic/transcription feedback loop, in which NAD⁺ levels determine the oscillatory synthesis of Nampt, the key enzyme in the NAD⁺ salvage pathway.

### Nampt Contributes to Circadian Control

The above considerations indicated that NAD⁺ directly influences SIRT1 to modulate circadian gene expression, and the circadian clock is itself controlling NAD⁺ levels by regulating Nampt gene transcription. If right, this scenario would predict that Nampt is critical in directing a modulated clock function. To address this question, FK866, a low molecular weight compound that specifically inhibits Nampt enzymatic activity and thereby lowers cellular NAD⁺ levels over prolonged length of time (30), was used. FK866 (also known as APO866 and WK175) is a specific competitive inhibitor designed based on the crystal structures of Nampt, alone and in complex with the reaction product nicotinamide mononucleotide. Structural studies have shown that FK866 is bound in a tunnel at the interface of the Nampt dimer, and competes directly with the nicotinamide substrate (40). Thus, FK866 was used to pharmacologically and specifically block Nampt function.

To assess the role of Nampt in circadian regulation, and thereby the contribution of oscillatory levels of NAD⁺ to the clock, the circadian profile of Per2 and Dbp gene expression were compared in serum-stimulated MEFs treated or not with FK866. It was observed that blocking Nampt enzymatic activity significantly modifies circadian regulation (FIG. 16A). In MEFs pretreated with FK866 and then serum-stimulated, the onset of the circadian peak for both genes is earlier by 3-4 hours and the amplitude of oscillation increases by an average of 30-40%. Importantly, blocking Nampt function with FK866 elicits a circadian imbalance highly reminiscent of the one the inventor has observed when SIRT1 is inhibited by the specific inhibitors nicotinamide and splitomicin in cultured cells or in Sirt1-deficient mice and MEFs (see Example 1 above, also see ref. 8). Based on this consideration, it was predicted that blocking Nampt would cause a change in the acetylation profile of CLOCK-SIRT1 targets, in a manner that could parallel the effect of inhibiting the HDAC activity of SIRT1. To analyze this possibility the anti-acetyl-BMAL1 antibody recently developed was used (see Example 1 above, also see ref. 8). BMAL1 is a specific target of CLOCK mediated acetylation and SIRT1-mediated deacetylation at the unique, highly conserved lysine residue K537 (see Example 1 above, also see refs. 7, 8). Importantly, BMAL1 acetylation at K537 is critical for circadian physiology (7). Treatment of cultured MEFs with FK866 has no major effect on BMAL1 protein levels at any time of the circadian cycle, while it has a drastic effect on K537 acetylation. Indeed, BMAL1 acetylation is heavily increased and displays a broader peak (FIG. 16B). Significantly, this BMAL1 acetylation profile is basically equivalent to the one observed in MEFs and livers from Sirt1−/− mice (see Example 1 above, also see ref 8).

### Concluding Remarks

In essence, the circadian core regulators CLOCK:BMAL1 modulate the circadian expression of the Nampt gene, which in turn controls the intracellular levels of NAD⁺, a key metabolite that functions as coenzyme of SIRT1. The NAD⁺ levels oscillate in a circadian manner, thereby inducing the HDAC activity of SIRT1 in a rhythmic fashion. CLOCK:BMAL1 and SIRT1 are in the same transcription/chromatin remodeling complex, and together control target promoters, specifically inducing the oscillatory expression of the Nampt gene (FIG. 14). Thus, the results reveal the interlocking of two auto-regulatory systems, in which a classical transcription circadian loop is coupled to an enzymatic feedback loop (FIG. 16C). Blocking Nampt enzymatic activity with the specific FK866 inhibitor directly and uniquely affects the NAD⁺ salvage pathway, without altering the de novo NAD⁺ biosynthesis. Importantly, the significant effect of FK866 on circadian physiology (FIG. 16) parallels the effect recently described of inhibiting SIRT1. Without wishing to be bound by any specific theory, this points to the oscillation of NAD⁺ as a key regulatory step in the modulation of rhythms. FK866 can be used for treatment of diseases implicating deregulated apoptosis such as cancer, for immunosuppression, or as a sensitizer for genotoxic agents. In addition, at least one other enzyme may be critically influenced by oscillatory NAD⁺ levels: the poly(ADP-ribose) polymerase-1 (PARP-1) utilizes NAD⁺ as coenzyme to exert its role in recovery from DNA damage, and a functional interplay between SIRT1 and PARP-1 has been reported. Thus, the findings reported here have multiple implications.

### References for Example 2

1. J. C. Dunlap, Cell 96, 271 (1999).
2. C. B. Green, J. S. Takahashi, J. Bass, Cell 134, 728 (2008).
3. B. P. Tu, S. L. McKnight, Nat Rev Mol Cell Biol 7, 696 (2006).
4. U. Schibler, P. Sassone-Corsi, Cell 111, 919 (2002).
5. H. Wijnen, M. W. Young, Annu Rev Genet. 40, 409 (2006).
6. M. Doi, J. Hirayama, P. Sassone-Corsi, Cell 125, 497 (2006).
7. J. Hirayama et al., Nature 450, 1086 (2007).
8. Y. Nakahata et al., Cell 134, 329 (2008).
9. G. Asher et al., Cell 134, 317 (2008).
10. J. Landry et al., Proc Natl Acad Sci USA 97, 5807 (2000).
11. J. S. Smith et al., Proc Natl Acad Sci USA 97, 6658 (2000).
12. S. Imai, C. M. Armstrong, M. Kaeberlein, L. Guarente, Nature 403, 795 (2000).
13. K. J. Bitterman, R. M. Anderson, H. Y. Cohen, M. Latorre-Esteves, D. A. Sinclair, J Biol Chem 277, 45099 (2002).
14. S. J. Lin, E. Ford, M. Haigis, G. Liszt, L. Guarente, Genes Dev 18, 12 (2004).
15. R. M. Anderson, K. J. Bitterman, J. G. Wood, O. Medvedik, D. A. Sinclair, Nature 423, 181 (2003).
16. L. Bordone, L. Guarente, Nat Rev Mol Cell Biol 6, 298 (2005).
17. A. Rongvaux, F. Andris, F. Van Gool, O. Leo, Bioessays 25, 683 (2003).
18. J. R. Revollo, A. A. Grimm, S. Imai, J Biol Chem 279, 50754 (2004).
19. R. M. Anderson et al., J Biol Chem 277, 18881 (2002).
20. E. van der Veer et al., J Biol Chem 282, 10841 (2007).
21. A. Rongvaux et al., J Immunol 181, 4685 (2008).
22. H. Yang et al., Cell 130, 1095 (2007).
23. S. K. Bae et al., FEBS Lett 580, 4105 (2006).
24. S. Ognjanovic et al., J Mol Endocrinol 26, 107 (2001).
25. A. Balsalobre, F. Damiola, U. Schibler, Cell 93, 929 (1998).
26. J. Wang et al., J Cell Biol 170, 349 (2005).
27. K. Yamada, N. Hara, T. Shibata, H. Osago, M. Tsuchiya, Anal Biochem 352, 282 (2006).
28. M. H. Vitaterna et al., Science 264, 719 (Apr. 29, 1994).
29. R. A. Billington, A. A. Genazzani, C. Travelli, F. Condorelli, Autophagy 4, 385 (2008).
30. M. Hasmann, I. Schemainda, Cancer Res 63, 7436 (2003).
31. U. H. Olesen et al., Biochem Biophys Res Commun 367, 799 (2008).
32. J. R. Revollo et al., Cell Metab 6, 363 (2007).
33. H. Ando et al., Endocrinology 146, 5631 (2005).
34. K. F. Storch et al., Nature 417, 78 (2002).
35. H. R. Ueda et al., Nature 418, 534 (2002).
36. http://expression.gnforg/cgi-bin/circadian/index.cgi
37. H. R. Ueda et al., Nat Genet. 37, 187 (2005).
38. T. Yamamoto et al., BMC Mol Biol 5, 18 (2004).
39. D. E. Nowak, B. Tian, A. R. Brasier, Biotechniques 39, 715 (2005).
40. J. A. Khan, X. Tao, L. Tong, Nat Struct Mol Biol 13, 582 (2006).
41. T. Alenghat et al., Nature 456, 997 (2008).
42. C. Liu, S. Li, T. Liu, J. Borjigin, J. D. Lin, Nature 447, 477 (2007).
43. X. Yang et al., Cell 126, 801 (2006).
44. F. Damiola et al., Genes Dev 14, 2950 (2000).
45. K. Kaasik, C. C. Lee, Nature 430, 467 (2004).
46. K. A. Lamia, K. F. Storch, C. J. Weitz, Proc Natl Acad Sci USA 105, 15172 (2008).
47. F. W. Turek et al., Science 308, 1043 (2005).
48. L. Virag, C. Szabo, Pharmacol Rev 54, 375 (2002).
49. U. Kolthur-Seetharam, F. Dantzer, M. W. McBurney, G. de Murcia, P. Sassone-Corsi, Cell Cycle 5, 873 (2006).
50. J. B. Pillai, A. Isbatan, S. Imai, M. P. Gupta, J Biol Chem 280, 43121 (2005).
51. L. Cardone et al., Science. 309,1390 (2005).
52. E. Borrelli et al., Neuron 60, 961 (2008).
53. K. Eckel-Mahan, P. Sassone-Corsi, Nature Struct. Mol. Biol. 16, 462 (2009).
54. Y. Nakahata et al., Science 324, 654 (2009).

### Example 3

### Use of SRT1720 and SRT2183 to Elucidate the Physiological Role of SIRT1 in Circadian Rhythms

We have used the SRT1720 and SRT2183 compounds in vivo (in the mouse) and in vitro (cultured mouse embryo fibroblasts) to establish whether inappropriate activation of SIRT1 would modulate circadian gene expression, possibly generating an outcome that would parallel the loss-of-function approach used in previous reports (see Example 1). Gene expression (in vitro data with SRT2183) and chromatin IP (from in vivo tissues, with SRT1720) results show that SIRT1 modulates the amplitude of circadian response and dictates the recruiting of CLOCK-BMAL1 to target promoters.

The effect of SRT2183 on circadian clock expression is shown in FIG. 19. This figure shows mDbp and mNampt mRNA expression profile in MEFs either treated with SRT2183 or not, analyzed by quantitative PCR.

WT MEFs were cultured in Dulbecco's Modified Eagle Medium (DMEM) supplemented with 10% fetal bovine serum (FBS) and antibiotics until they became confluent. Cells were pre-treated with 10 mM SRT2183 dissolved in DMSO (final concentration of 0.25%) for 16 hrs, prior to high serum treatment. The same vehicle (same % of DMSO without SRT compound) was used as negative control.

After 2 hr serum shock (DMEM+50% horse serum), cells were maintained in serum-free medium and harvested every 6 hrs. RNA extractions were done using TRIzol reagent (GIBCO, BRL). Quantitative Real-Time RT-PCR was performer using the Chromo4 real time detection system (BIO-RAD). Primers for mDbp and mNampt were designed with a real-time PCR primer design tool. The reaction was performed by mixing 50 ng of cDNA template with the primers (final concentration of 200 nM) and 10 ul of iQ™ SYBR Green Supermix (BIO-RAD). The reaction was first incubated to 95° C. for 3 min, followed by 40 cycles at 95° C. for 30 s and 60° C. for 1 min. Results obtained were the average of three different experiments.

Similar results were obtained through transient treatment for 1 hr at time 15 after serum shock.

The effect of SRT1720 on circadian clock control is shown in FIG. 20. This figure shows histone H3 (Lys9/Lys14) acetylation and CLOCK recruitment at the E-box elements within the Dbp promoter in liver samples from WT and liver specific KO mice (LKO), either treated or not with SRT1720.

For this experiment we used tissue-specific Sirt1−/− mice in which the loxed gene (exon 4 of the gene that contains the SIRT1 catalytic domain) was selectively deleted by albumin promoter-driven Cre recombinase in the liver. Either WT and LKO mice were fed a standard diet supplemented or not with SRT1720 for three weeks. The compound was suspended in 40% PEG400/0.5% Tween 80 in deionized water and incorporated into the diet (Research Diets). The average dosage for each animal was 100 mg SRT1720/kg body weight/day. The same vehicle (dosing solution without SRT compound) was used as negative control group.

After three weeks, livers were collected at different times of the circadian cycle (Zeitgeber times, ZT) and processed following the Double cross-linking Chromatin Immunoprecipitation (ChIP) assay protocol. The liver samples were double cross linked by using a solution of Disuccinimidyl Glutalate (DSG) in PBS 1× to a final concentration of 2 mM for 45 minutes, followed by incubation at room temperature with 1% (v/v) formaldehyde solution in PBS 1×. After stopping the reaction with glycine (0.1 M final concentration), samples were homogenized and nuclear extraction was performed. Samples were sonicated to obtain DNA fragments 100-600 bp in length. ChIP was performed by using anti-acetyl histone H3 (Lys9/Lys14), anti-CLOCK antibodies and control IgG and analyzed by semiquantitative RT-PCR with DBP up primers, that amplified a region containing E-boxes in the Dbp promoter. Three mice for each condition (WT vs LKO; vehicle vs compound; ZT3 vs ZT15) were used. Representative results are shown.

### EQUIVALENTS

Thus, specific embodiments and applications of compositions and methods related to SIRT1 function have been disclosed. It should be apparent, however, to those skilled in the art that many more modifications besides those already described are possible without departing from the inventive concepts herein. The inventive subject matter, therefore, is not to be restricted except in the spirit of the present disclosure. Moreover, in interpreting the specification and contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Those skilled in the art will recognize, or be able to ascertain using no more than routine experimentation, many equivalents of the specific embodiments of the invention described herein. Such equivalents are intended to be encompassed by the following claims.

What is claimed is:

1. A method, comprising
administering to a subject having a disease or disorder associated with a circadian rhythm dysfunction and in need of such treatment an agent that modulates SIRT1 activity or expression or that modulates binding of SIRT1 to CLOCK or CLOCK/BMAL, in an amount effective to modulate the SIRT1 activity or expression or the binding of SIRT1 to CLOCK or CLOCK/BMAL.

2. The method of claim 1, wherein the disease or disorder is a sleep disorder.

3. The method of claim 2, wherein the sleep disorder is insomnia, jet lag, shift work sleep disorder, delayed sleep phase syndrome (DSPS), advanced sleep phase syndrome (ASPS), non 24-hour sleep wake disorder or irregular sleep-wake pattern.

4. The method of claim 1, wherein the disease or disorder is a psychiatric disorder associated with circadian rhythm.

5. The method of claim 4, wherein the psychiatric disorder is depression.

6. The method of claim 1, wherein the disease or disorder is a neurological disease with a circadian rhythm component.

7. (canceled)

8. The method of claim 1, wherein the disease or disorder is selected from the group consisting of anorexia nervosa, abnormal blood pressure, abnormal heart rate and asthma.

9.-13. (canceled)

14. The method of claim 1 wherein treating comprises ameliorating symptoms of the disease or disorder.

15. The method of claim 1, wherein modulating comprises changing the amplitude of a molecular oscillation associated with the circadian clock.

16. The method of claim 15, wherein the molecular oscillation is an activation and/or inhibition of gene expression and/or gene product function.

17.-18. (canceled)

19. The method of claim 16, wherein the activation and/or inhibition of gene expression and/or gene product function is mediated by an acetylation, phosphorylation, and/or methylation of a protein, wherein the protein is BMAL1 or PER2.

20. The method of claim 19, wherein the post-translational modification is acetylation of lysine 537 of BMAL1 and/or acetylation of PER2.

21. The method of claim 1, wherein the agent increases deacetylation of a member of the CLOCK/BMAL1 pathway.

22. The method of claim 1, wherein the agent increases the binding of SIRT1 to a member of the CLOCK/BMAL1 pathway and/or SIRT1 deacetylase activity.

23. The method of claim 22, wherein the increase is mediated by an increase in SIRT1 expression.

24. The method of claim 1, wherein the agent is a non-naturally occurring compound.

25. The method of claim 24, wherein the agent is SRT1720, SRT2183, or SRT1460.

26.-28. (canceled)

29. The method of claim 1, wherein the method includes first testing the subject to determine if the subject's disease or disorder has a circadian rhythm component.

30. A method for treating a disease or disorder that has a circadian rhythm component comprising
determining whether the circadian rhythm of a subject is disrupted, and
administering to the subject in need of such treatment an agent that modulates SIRT1 activity or expression or that modulates binding of SIRT1 to CLOCK or CLOCK/BMAL, in an amount effective to treat the disease or disorder.

31. A method for altering a circadian rhythm of a subject comprising
administering to the subject in need of such treatment an amount of a SIRT1 modulator (activator or inhibitor) effective to alter the circadian rhythm of the subject.

32. The method of claim 31, wherein the method is used for treating disrupted sleep patterns of the subject.

33. The method of claim 31, wherein the method is used for initiating the onset of sleep or prolonging a period of sleep in the subject.

34. The method of claim 31, wherein the method is used for increasing the level of alertness in the subject.

35. The method of claim 31, wherein the method is used for extending wakefulness of the subject.

36. The method of claim 31, wherein the method is used for increasing the rate of metabolism of the subject.

37.-39. (canceled)

40. The method of claim 31, wherein the SIRT1 modulator is a non-naturally occurring compound.

41. The method of claim 40, wherein the SIRT1 modulator is SRT1720, SRT2183, or SRT1460.

42. (canceled)

43. (canceled)

44. A method for modulating CLOCK acetylase activity or BMAL acetylation in a eukaryotic cell comprising
modulating the expression or activity of SIRT1.

45. A method of identifying modulators of the circadian rhythm, comprising
contacting a cell that expresses SIRT1, CLOCK and BMAL with a candidate molecule, and
determining the level, activity or acetylation state of a biomarker indicating circadian rhythm activity,
wherein if the level or acetylation state of the biomarker in the cell that has been contacted with the candidate molecule differs from a reference or control level of the level or acetylation state of the biomarker, then the candidate molecule is a modulator of the circadian rhythm.

46. An isolated antibody that specifically binds to BMAL1 acetylated at lysine 537, or an antigen binding fragment of the antibody.

47.-52. (canceled)

53. A method of modulating a process that is at least in part regulated by CLOCK:BMAL1, comprising:
identifying the process in a cell as being regulated at least in part by CLOCK:BMAL1; and
exposing the cell to an agent that modulates at least one of Nampt expression and Nampt activity at a concentration effective to modulate the at least one of Nampt expression and Nampt activity.

54. A method, comprising
administering to a subject having a disease or disorder associated with deregulated apoptosis and in need of such treatment an agent that inhibits Nampt expression or function, in an amount effective to inhibit the Nampt expression or function.

55. The method of claim 54, wherein the disease or disorder is cancer.

56.-59. (canceled)

60. The method of claim 54, wherein the agent is FK866.

61. The method of claim 54, wherein the agent is a siRNA molecule that reduces expression of Nampt expression.

62. (canceled)

63. (canceled)

64. The method of claim 54, wherein the method includes first testing the subject to determine if the subject's disease or disorder has a circadian rhythm component.
